(19) United States
(12) Patent Application Publication
(10) Pub. No.: US 2023/0126276 A1
(43) Pub. Date: Apr. 27, 2023

(54) FENTANYL HAPTENS, FENTANYL HAPTEN CONJUGATES, AND METHODS FOR MAKING AND USING

(71) Applicants: REGENTS OF THE UNIVERSITY OF MINNESOTA, Minneapolis, MN (US); ALLEGHENY-SINGER RESEARCH INSTITUTE, Pittsburgh, PA (US); RTI INTERNATIONAL, Research Park, NC (US)

(72) Inventors: Marco Pravetoni, Minneapolis, MN (US); Saadyah Averick, Pittsburgh, PA (US); Scott Runyon, Research Park, NC (US)

(21) Appl. No.: 17/909,874

(22) PCT Filed: Mar. 12, 2021
(86) PCT No.: PCT/US2021/022148

§ 371 Date: Sep. 7, 2022

Related U.S. Application Data
(60) Provisional application No. 62/989,417, filed on Mar. 13, 2020.

(51) Int. Cl. A61K 39/385; A61K 31/4468; A61K 39/00; A61K 47/64

(52) U.S. Cl. CPC A61K 39/385; A61K 31/4468; A61K 39/0013; A61K 47/643; A61K 47/646

(57) ABSTRACT

This disclosure describes fentanyl haptens, a fentanyl hapten-carrier conjugate, methods of making the fentanyl hapten-carrier conjugate, and methods of using the fentanyl hapten-carrier conjugate including, for example, as a prophylactic vaccine to counteract toxicity from exposure to fentanyl, fentanyl derivatives, and fentanyl analogs. In some embodiments, the fentanyl hapten-carrier conjugate or a composition including the fentanyl hapten-carrier conjugate may be used in an anti-opioid vaccine.

### CROSS-REFERENCE TO RELATED APPLICATIONS

This application claims the benefit of U.S. Provisional Application Ser. No. 62/989,417, filed Mar. 13, 2020, which is incorporated by reference herein.

### GOVERNMENT FUNDING

This invention was made with government support under DA048386 awarded by the National Institutes of Health. The government has certain rights in the invention.

### BACKGROUND

Opioid use disorders (OUDs) and the fatal overdose epidemic are a growing public health burden. The incidence of fatal overdoses from synthetic opioids increased 100% from 2015 to 2016, largely driven by illicitly manufactured fentanyl. Fentanyl, an extremely potent synthetic opioid, and its analogs have been involved in more than 50% of opioid-related fatalities in the United States. Fentanyl has been increasingly used to adulterate heroin, cocaine, and counterfeit prescription pills, leading to an increase in opioid-induced fatal overdoses in the United States, Canada, and Europe.

Fentanyl analogs have also been used by Russian Special Forces in a Moscow theater hostage situation, which resulted in at least 150 fatal overdoses in both civilian and terrorists. It is feared that fentanyl and its analogs may be used in mass casualty incidents, deliberate poisoning, and chemical attacks against civilians, military, and at-risk professionals.

Current pharmacotherapies are not sufficient to address the current epidemic of OUDs and opioid-related overdoses.

### SUMMARY OF THE INVENTION

Vaccines offer a novel strategy to reduce and prevent toxicity from deliberate and accidental exposure to fentanyl and or a fentanyl derivative (also referred to herein as a fentanyl analog). In one aspect, this disclosure describes vaccine formulations containing a hapten derived from fentanyl or its analogs conjugated to a carrier (also referred to herein as, a fentanyl hapten-carrier conjugate).

In another aspect, this disclosure describes the development of new haptens derived from fentanyl or its analogs. In a further aspect, this disclosure describes fentanyl hapten-carrier conjugates, methods of making the fentanyl hapten-carrier conjugates, and methods of using the fentanyl hapten-carrier conjugates including, for example, as a prophylactic or a therapeutic vaccine to counteract toxicity from exposure to fentanyl or a fentanyl derivate.

As used herein, “fentanyl” in a fentanyl hapten-carrier conjugate refers to fentanyl or a fentanyl derivative or both; that is, as further described herein, the fentanyl hapten of a fentanyl hapten-carrier conjugate may be derived from fentanyl or from a fentanyl derivative.

As used herein a “fentanyl derivative” (also referred to herein as a “derivative of fentanyl” or a “fentanyl analog”) includes analogs of fentanyl such as acetylfentanyl, alfentanil, brifentail, carfentanil, lofentanil, mefentanyl, α-mefentanyl, mirfenantil, ohmefentanyl, phenaridine, remifentanil, sufentanil, trefentanil, etc.

As used herein, room temperature (RT) or “ambient” temperature refers to a temperature in a range of 15° C. to 25° C.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the invention.

The terms “comprises” and variations thereof do not have a limiting meaning where these terms appear in the description and claims. Such terms will be understood to imply the inclusion of a stated step or element or group of steps or elements but not the exclusion of any other step or element or group of steps or elements.

By “consisting of” is meant including, and limited to, whatever follows the phrase “consisting of” Thus, the phrase “consisting of” indicates that the listed elements are required or mandatory, and that no other elements may be present. By “consisting essentially of” is meant including any elements listed after the phrase and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

Unless otherwise specified, “a,” “an,” “the,” and “at least one” are used interchangeably and mean one or more than one.

As used herein, the term “or” is generally employed in its usual sense including “and/or” unless the content clearly dictates otherwise.

The term “and/or” means one or all of the listed elements or a combination of any two or more of the listed elements.

Also herein, the recitations of numerical ranges by endpoints include all numbers subsumed within that range (for example, 1 to 5 includes 1, 1.5, 2, 2.75, 3, 3.80, 4, 5, etc.).

For any method disclosed herein that includes discrete steps, the steps may be conducted in any feasible order. And, as appropriate, any combination of two or more steps may be conducted simultaneously.

All headings are for the convenience of the reader and should not be used to limit the meaning of the text that follows the heading, unless so specified.

Reference throughout this specification to “one embodiment,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1A shows the structure of fentanyl, carfentanil, and alfentanil. FIG. 1B shows a series of fentanyl-based haptens F₁ to F₈. FIG. 1C shows a series of fentanyl-based haptens F₉ₐ to F₁₃. The first-generation fentanyl-based F₁ hapten (also referred to as F(Gly)₄) includes a tetraglycine linker. Second-generation haptens F₂₋₁₃ are described and characterized in this disclosure. FIG. 1D shows Scheme 1, synthesis of the F₁ hapten (structure 3), as further described in Example 1. FIG. 1E shows synthesis of the F₂ hapten, as further described in Example 1. FIG. 1F shows synthesis of the F₃ hapten (structure 6), as further described in Example 1. FIG. 1G shows synthesis of the F₄ (structure 8), F₅ (structure 6b), and F₆ (structure 6a) haptens, as further described in Example 1. “a” indicates meta and “b”=para for structures 1-6. FIG. 1H shows synthesis of the F₇ hapten (11), as further described in Example 1. FIG. 1I shows synthesis of the F₈ hapten (11), as further described in Example 4. FIG. 1J shows synthesis of the F₉ₐ (amino fentanyl) hapten, as further described in Example 3. FIG. 1K shows synthesis of the F_{9b} (carboxylic acid fentanyl) hapten, as further described in Example 3. FIG. 1L shows synthesis of the F₁₀ hapten, as further described in Example 4. FIG. 1M shows synthesis of the F₁₁ hapten, as further described in Example 4. FIG. 1N shows synthesis of the F₁₂ hapten, as further described in Example 4. FIG. 1O shows synthesis of the F₁₃ hapten, as further described in Example 4. FIG. 1P-FIG. 1Q shows the series of fentanyl-based haptens F₁₋₉ conjugated to a carrier. FIG. 1R shows a divalent conjugate vaccine containing both fentanyl- and carfentanil-based haptens attached to an E. coli-expressed CRM carrier protein via a novel dual labeling strategy involving sequential reaction of fentanyl to lysine residues and carfentanil to aspartic/glutamic acid residues using activated water or equivalent coupling.

FIG. 2A-FIG. 2E show vaccine efficacy against fentanyl in mice. Conjugates including the F₁₋₇ haptens were tested in two independent cohorts of BALB/c mice. Haptens were conjugated to either sKLH, CRM₁, or CRM₂, adsorbed on aluminum adjuvant (alum) and injected intramuscularly (IM) in mice on days 0, 14 and 28. FIG. 2A. A week after the last immunization, the first cohort of mice receiving F₁₋₆ was challenged with 0.05 mg/kg subcutaneous (s.c.) fentanyl. Conjugates were effective in reducing fentanyl-induced antinociception in the hot plate test at 30-minutes post-challenge with a p≤0.0001. FIG. 2B. A second cohort of mice immunized with conjugates containing the F₁, F₃ and F₇ haptens were challenged with 0.1 mg/kg s.c. fentanyl and showed reduced fentanyl-induced antinociception in the hot plate test (with the exception of F₇-sKLH). The F₇ hapten is derived from carfentanil and shows that there is no cross reactivity between fentanyl and carfentanil. Blood and brain were collected from mice immunized with F₁, F₃, F₄, F₅, F₆, or F₇ haptens and selectively showed increasing retention of fentanyl in serum (FIG. 2C) or decreasing distribution of fentanyl to the brain (FIG. 2D-FIG. 2E). Statistical symbols: *p≤0.05, **p≤0.01, and ****p≤0.0001 compared to control.

FIG. 3A-FIG. 3F show vaccine efficacy against fentanyl in rats. Conjugates containing the F₁₋₃ haptens conjugated to either sKLH, CRM₁ or CRM₂ were tested in Sprague Dawley rats. Conjugates were injected IM on days 0, 21, 42, and 63. A week after the third vaccination, rats were challenged weekly with either fentanyl (FIG. 3) or sufentanil (FIG. 4). On week 1, conjugates were effective in reducing effects of 0.075 mg/kg s.c. fentanyl:antinociception in the hot plate test (FIG. 3A), respiratory depression reported as percentage (%) of oxygen saturation measured by oximetry (FIG. 3B), and bradycardia reported as heart rate measured by oximetry (FIG. 3C). On week 3, rats were challenged with 0.1 mg/kg s.c. fentanyl and total fentanyl in the brain was measured at 60 minutes. Conjugates, F₁-sKLH to a lesser extent, were effective in reducing fentanyl-induced respiratory depression (FIG. 3D), bradycardia (FIG. 3E), and distribution of fentanyl to the brain (FIG. 3F). Statistical symbols: *p≤0.05, **p≤0.01, ***p≤0.001, ****p≤0.0001 compared to control.

FIG. 4A-FIG. 4D show vaccine efficacy against sufentanil in rats. On week 2, rats immunized with conjugates containing the F₁₋₃ haptens were challenged with 0.008 mg/kg s.c. sufentanil. Selected conjugates containing F₁, F₂ and F₃ haptens were effective in reducing sufentanil-induced antinociception in the hot plate test measured at 15 minutes (FIG. 4A), 30 minutes (FIG. 4B), 45 minutes (FIG. 4C), and 60 minutes (FIG. 4D) post-drug challenge. These rats are the same subjects as shown in FIG. 3. Statistical symbols: * or #p≤0.05, ** or ##p≤0.01, *** or ###p≤0.001 compared to either control (*) or F₁-sKLH (#).

FIG. 5A-FIG. 5F shows efficacy of vaccines containing haptens F₄₋₆ against fentanyl and sufentanil in rats. Conjugates containing the F₄₋₆ haptens conjugated to either sKLH or CRM₂ were tested in rats. Conjugates were injected IM on days 0, 21, 42, and 63. A week after the 3^{rd} vaccination, rats were challenged weekly with either 0.1 mg/kg s.c. fentanyl or 0.008 mg/kg s.c. sufentanil. All conjugates were effective in reducing effects of fentanyl:antinociception in the hot plate test (FIG. 5A), respiratory depression reported as percentage (%) of oxygen saturation measured by oximetry (FIG. 5B), and bradycardia reported as heart rate measured by oximetry (FIG. 5C). FIG. 5D. F₆-CRM₂ was effective in reducing effects of sufentanil antinociception in the hot plate test. Multiple conjugates showed a trend towards reduced effects in respiratory depression (FIG. 5E) and bradycardia (FIG. 5F). Statistical symbols: * or #p≤0.05, **p≤0.01, ***p≤0.001, ****p≤0.0001 compared to either control (*) or F₁-sKLH (#).

FIG. 6A-FIG. 6B show active immunization reduced fentanyl intravenous self-administration (FSA) in rats. Rats were trained to self-administer fentanyl (1 μg/kg/infusion) under a fixed-ratio (FR) 3 schedule during daily 120-min sessions. Once FSA stabilized, rats were immunized IM with either CRM₁ or F₁-CRM₁ (n=6/group) on days 0, 21, 42, and 63. FIG. 6A. FSA declined by more than 50% in the F₁-CRM₁ group after the 4^{th} immunization compared to its baseline level prior to vaccination (47.7±11.7 mean±SEM), whereas the control group showed no change. FIG. 6B. When rats were challenged with a dose-reduction protocol, rats vaccinated with F₁-CRM₁ decreased their intake overtime, while control CRM₁ increased their mean infusion/session to compensate for the dose reduction. Statistical symbols: * and #p≤0.05 compared to control or pre-immunization baseline.

FIG. 7A-FIG. 7K show immunization against fentanyl does not interfere with induction of anesthesia (using demedetomidine) and rescue from anesthesia (using atipamezole). Sprague Dawley rats were immunized i.m. with either CRM₁ or F₁-CRM₁ (n=6, each group) on days 0, 21, 42 and 63. From day 49, rats were challenged weekly with 2% inhaled isoflurane, 0.25 mg/kg dexmedetomidine (reversed by 1 mg/kg atipamezole), 75 mg/kg ketamine, 100 mg/kg propofol, or 0.1 mg/kg fentanyl as control. Anesthetic efficacy was measured by induction time reported as the latency of the loss of righting reflex, respiratory depression reported as percentage (%) of oxygen saturation and bradycardia reported as heart rate both measured by oximetry. Results were equivalent in CRM₁ or F₁-CRM₁ groups, confirming the selectivity of these anti-fentanyl vaccines. FIG. 7A-FIG. 7C show dexmedetomidine (reversed by 1 mg/kg atipamezole), FIG. 7D-FIG. 7F show fentanyl control, FIG. 7G-FIG. 7H show ketamine, FIG. 7I shows propofol, and FIG. 7J-FIG. 7K shows isofluorane.

FIG. 8A-FIG. 9D show representative MALDI-TOF and Dynamic Light Scattering (DLS) characterization of conjugates (F₁-BSA, F₁-sKLH, F₁-CRM) and unconjugated carrier proteins (BSA, sKLH, CRM). FIG. 8A shows representative MALDI-TOF traces of BSA and F₁-BSA with an haptenization ration (HR) of 23. Representative DLS traces of F₁-sKLH (FIG. 8B), F₁-CRM₁ (FIG. 8C), and F₁-CRM₂ (FIG. 8D). FIG. 8B-FIG. 8D show conjugates at T=0 and 1 month after storage at +4° C.

FIG. 9A shows the F₁ hapten does not contain the N-phenylethyl moiety that is critical for activity at the MOR but is replaced with a tetraglycine peptidic linker that yields in a final hapten that has no functional activity at the MOR. FIG. 9B shows the F₁ hapten has no functional agonist activity at the MOR, most likely due to the extended peptidic linker and lack of an N-phenylethyl substituent. Having haptens that lack the N-phenethyl moiety results in compounds that are devoid of MOR activity, which is expected to increase their safety profile.

FIG. 10A shows a visual representation of the conjugation of F₇ to sKLH. FIG. 10B shows a visual representation of conjugation of F₇ and BSA.

FIG. 11 shows the conjugation of F₃ and F₇ to PEG and biotin. These reagents can be attached to streptavidin or streptavidin conjugated to pychoerytrin (PE) and used as either coating antigens for ELISA or as baits to sort or analyze opioid-specific B cells as described in Taylor J J et al. J. Immunol. Methods, 2014; 405:74-86 or Laudenbach et al., J. Immunol. 2015; 194(12):5926-36. These reagents can additionally or alternatively be used to isolate and analyze B cells specific for fentanyl and its analogs in mouse, rat, human, and other species of pre-clinical and clinical interest.

FIG. 12A-FIG. 12D show efficacy of vaccines containing haptens F₄₋₆ against a high dose of fentanyl in rats. Conjugates containing the F₄₋₆ haptens conjugated to either sKLH or CRM₂ were tested in Sprague Dawley rats (n=6, each group). Conjugates were injected i.m. on days 0, 21, 42, and 63. After being challenged with fentanyl and sufentanil (0.1 and 0.008 mg/kg, respectively) as shown in FIG. 5A-FIG. 5F, rats were challenged with a final dose of fentanyl (1 mg/kg, s.c.) and monitored via pulse oximetry at 15 minutes post-drug challenge. FIG. 12A shows pre- and post-challenge breath rate; FIG. 12B shows breath rate expressed as a percent reduction from baseline; FIG. 12C shows brain fentanyl concentrations; and FIG. 12D shows the ratio of fentanyl in the brain versus serum as measured by LC/MS. Symbols: *, **, ***, **** indicate p≤0.05, 0.01, 0.001, 0.0001, respectively, compared to control.

FIG. 13A-FIG. 13F show efficacy of the F_{1, 8-10} haptens against fentanyl and sufentanil in rats. Sprague Dawley rats (n=6, each group) were vaccinated i.m. on days 0, 21, 42, and 63 with conjugates containing the F₁ and F₈₋₁₀ haptens conjugated to CRM₁ or CRM₂ and starting on day 49 challenged with 0.1 mg/kg s.c. fentanyl: FIG. 13A shows antinociception in the hot plate test reported as Maximal Possible Effect (MPE) %; FIG. 13B shows respiratory depression reported as oxygen saturation (%); and FIG. 13C shows bradycardia reported as heart rate measured by oximetry. Following challenges with fentanyl analogs, rats were vaccinated on day 63 and challenged on day 77 with 0.008 mg/kg s.c. sufentanil and monitored for drug-induced hot plate antinociception (FIG. 13D), respiratory depression (FIG. 13E), and bradycardia (FIG. 13F). Statistics for results of FIG. 13A-FIG. 13F are shown in Table 5A-Table 5F: *p≤0.05, **p≤0.01, ***p≤0.001, ****p≤0.0001, compared to control. Data are represented as mean±SEM.

FIG. 14A-FIG. 14F show efficacy of the F_{1, 8-10} haptens against alfentanil in rats. The same rats challenged in FIG. 13 were subsequently challenged with varying doses of alfentanil to further dissect differences in lead vaccine formulations. All rats (same as in the experiment described above) were first challenged with 0.5 mg/kg s.c. alfentanil: FIG. 14A shows antinociception in the hot plate test reported as MPE %, FIG. 14B shows respiratory depression reported as oxygen saturation (%), and FIG. 14C shows bradycardia reported as heart rate measured by oximetry. In a follow-up experiment, a subset of rats immunized with control, F₁-CRM₂, or F₈-CRM₂ were re-challenged with 0.25 mg/kg s.c. alfentanil: FIG. 14D shows antinociception in the hot plate test reported as MPE %, FIG. 14E shows respiratory depression reported as oxygen saturation (%), and FIG. 14F shows bradycardia reported as heart rate measured by oximetry. Statistics for results of FIG. 14A-FIG. 14C are shown in Table 6A-Table 6C: *p≤0.05, **p≤0.01, ***p≤0.001, ****p≤0.0001, compared to control. Data are represented as mean±SEM.

FIG. 15A-FIG. 15F show efficacy of vaccines containing the F_{1, 8-10} haptens against acetylfentanyl in rats. The same rats challenged in FIG. 13 and FIG. 14 were subsequently challenged with varying doses of acetylfentanyl to further dissect differences in lead vaccine formulations. All rats were first challenged with 0.5 mg/kg s.c. acetylfentanyl and measured for drug-induced hot plate antinociception (FIG. 15A), respiratory depression (FIG. 15B), and bradycardia (FIG. 15C). In a follow-up study, a subset of rats immunized with control, F₁-CRM₂, or F₁₀-CRM₁ were then re-challenged with 1 mg/kg s.c. acetylfentanyl and measured for drug-induced hot plate antinociception (FIG. 15D), respiratory depression (FIG. 15D), and bradycardia (FIG. 15F). Statistics for results of FIG. 15A-FIG. 15F are shown in Table 7A-Table 7F: *p≤0.05, **p≤0.01, ***p≤0.001, ****p≤0.0001, compared to control. Data are represented as mean±SEM.

FIG. 16A-FIG. 16C show efficacy of vaccines containing the F₁, F₁₁ and F₁₃ haptens against carfentanil in rats. Sprague Dawley rats (n=6 per group) were vaccinated i.m. on days 0, 21, 49, and 63 with conjugates containing the F₁ and F₁₁, F₁₃ haptens conjugated to CRM, or unconjugated CRM as control. On day 49, rats were challenged with 0.02 mg/kg s.c. carfentanil: FIG. 16A shows antinociception in the hot plate test reported as MPE %, FIG. 16B shows respiratory depression reported as oxygen saturation (%), and FIG. 16C shows bradycardia reported as heart rate measured by oximetry. Statistics for results of FIG. 16A-FIG. 16C are shown in Table 8A-Table 8C: *p≤0.05, **p≤0.01, ***p≤0.001, ****p≤0.0001, compared to control. Data are represented as mean±SEM.

FIG. 17A-FIG. 17C show efficacy of vaccines containing the F₁, F₁₁ and F₁₃ haptens against fentanyl in rats. The same rats as in the experiment described above were then challenged with 0.1 mg/kg s.c. fentanyl: FIG. 17A shows antinociception in the hot plate test reported as MPE %, FIG. 17B shows respiratory depression reported as oxygen saturation (%), and FIG. 17C shows bradycardia reported as heart rate measured by oximetry. Statistics for results of FIG. 17A-FIG. 17C are shown in Table 9A-Table 9C: *p≤0.05, **p≤0.01, ***p≤0.001, ****p≤0.0001, compared to control. Data are represented as mean±SEM.

FIG. 18A-FIG. 18C show efficacy of vaccines containing the F₁, F₁₁ and F₁₃ haptens against a combination of carfentanil and fentanyl in rats. The same rats as in the experiment described above (in FIG. 17) were then challenged with co-administration of 0.01 mg/kg s.c. carfentanil and 0.05 mg/kg s.c. fentanyl: FIG. 18A shows antinociception in the hot plate test reported as MPE %, FIG. 18B shows respiratory depression reported as oxygen saturation (%), and FIG. 18C shows bradycardia reported as heart rate measured by oximetry. Statistics for results of FIG. 18A-FIG. 18C are shown in Table 10A-Table 10C: *p≤0.05, **p≤0.01, ***p≤0.001, ****p≤0.0001, compared to control. Data are represented as mean±SEM.

FIG. 19A-FIG. 19C show efficacy of the F_{11/13} haptens against cumulative carfentanil dosing in rats. The same rats as in the experiments described above (FIG. 16-18) were challenged with 50 μg/kg carfentanil (s.c.) every 15 minutes to a final cumulative dose of 0.02 mg/kg: FIG. 19A shows antinociception in the hot plate rest reported as MPE %, FIG. 19B shows respiratory depression reported as oxygen saturation (%), and FIG. 19C shows bradycardia reported as heart rate measured by oximetry. Statistical symbols: *p≤0.05, **p≤0.01, ***p≤0.001, ****p≤0.0001 compared to control. Data are represented as mean±SEM.

FIG. 20A-FIG. 20D show efficacy of F₁, F₆, F₁₂ haptens against fentanyl in mice. Balb/c mice (n=4-6/group) were vaccinated i.m. on days 0, 14, and 21 with conjugates containing the F₁, F₆, F₁₂ haptens conjugated to CRM. On day 40 mice were challenged with 0.1 mg/kg s.c. fentanyl antinociception in the hot plate test reported as Maximal Possible Effect (MPE) % (FIG. 20A) and response latency (FIG. 20B). Vaccines containing F₁, F₆, F₁₂ haptens altered the distribution of fentanyl in the serum (FIG. 20C) and brain (FIG. 20D). F₁-CRM was more effective than F₆-CRM and F₁₂-CRM Statistical symbols: *p≤0.05, **p≤0.01, ***p≤0.001, ****p≤0.0001, compared to control. Data are represented as mean±SEM.

FIG. 21A shows the structures of fentanyl and alfentanil and the F₄, F₅, F₆, F₈ and F₁₂ haptens. Although each hapten retains its fentanyl or alfentanil-core structure, the hapten is equipped with different linkers attached at the para (F₄, F₅ and F₈) or meta (F₆ and F₁₂) position of the N-phenyl moiety. FIG. 21B shows the structures of fentanyl, alfentanil, carfentanil, and the F₁, F₉ₐ, F_{9b}, F₁₀, F₁₁, and F₁₃ haptens. Despite lacking what was considered to be a “critical” N-phenethyl moiety, each of these haptens raise a protective immune response that is effective and selective against their target compounds fentanyl, acetylfentanil and carfentanil in vivo. FIG. 21C-FIG. 21E shows typical opioid haptens at the time of the invention—each of which include the full structure of the target compound. FIG. 21C shows previously published carfentanil-based haptens (Eubanks et al. ACS Chem Biol 16, 277-282 (2021)). FIG. 21D shows a previously published fentanyl-based hapten (Barrientos et al. Mol Pharm 17, 3447-3460 (2020)). FIG. 21E shows an exemplary fentanyl-based hapten published by K D Janda and colleagues (Bremer et al. Angew Chem Int Ed Engl 55, 3772-3775 (2016), Smith et al. J Am Chem Soc 141, 10489-10503 (2019)).

### DETAILED DESCRIPTION OF ILLUSTRATIVE EMBODIMENTS

This disclosure describes fentanyl haptens, fentanyl hapten-carrier conjugates, methods of making the fentanyl hapten-carrier conjugates, and methods of using the fentanyl hapten-carrier conjugates including, for example, as a prophylactic or therapeutic vaccine to counteract toxicity and fatal overdose from deliberate or accidental exposure to fentanyl and its analogs and to prevent and treat opioid use disorders.

This disclosure also describes multivalent or multidisplay vaccine strategies, including methods of making a multivalent vaccine formulation, and methods of using a multivalent vaccine formulation including, for example, as a prophylactic or a therapeutic to counteract toxicity from exposure to fentanyl or a fentanyl derivative.

### Fentanyl

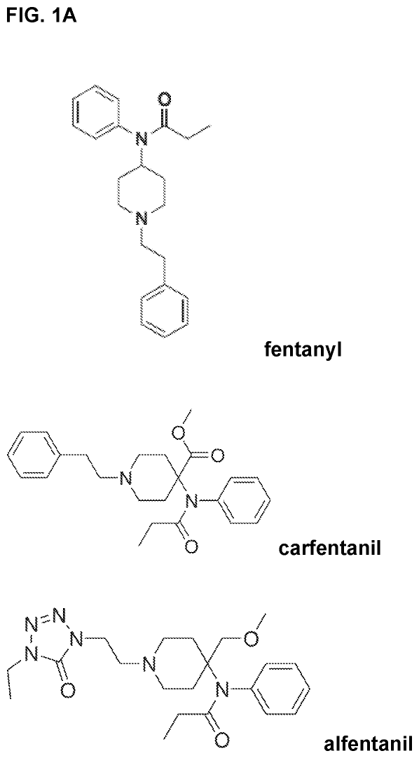

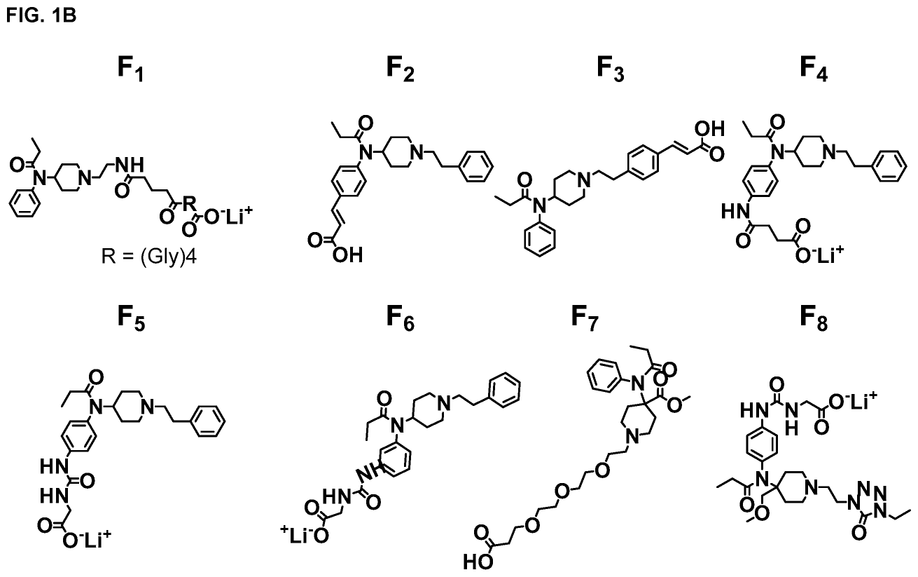

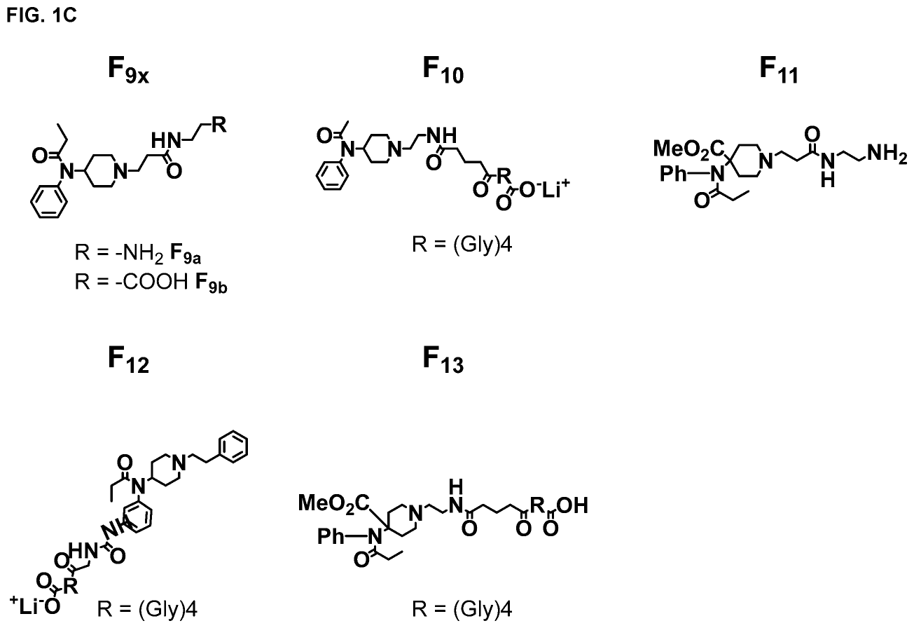

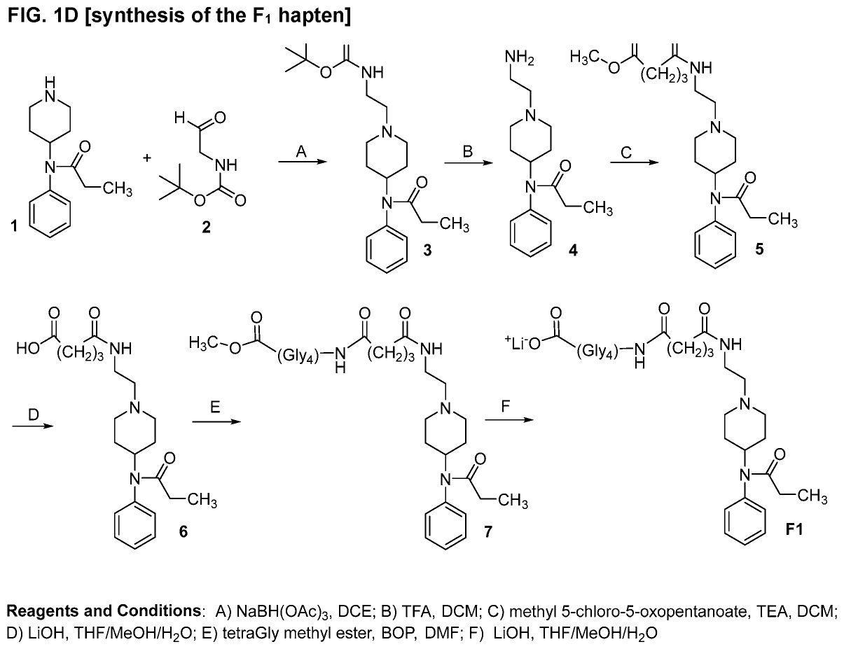

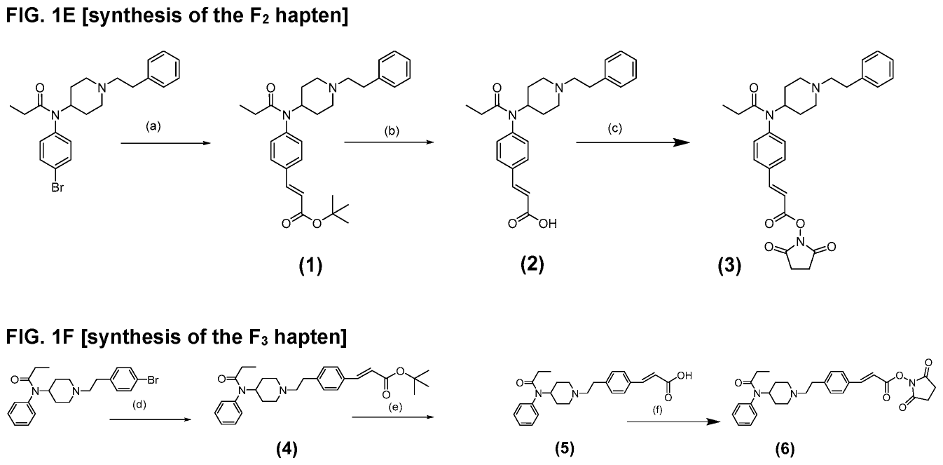

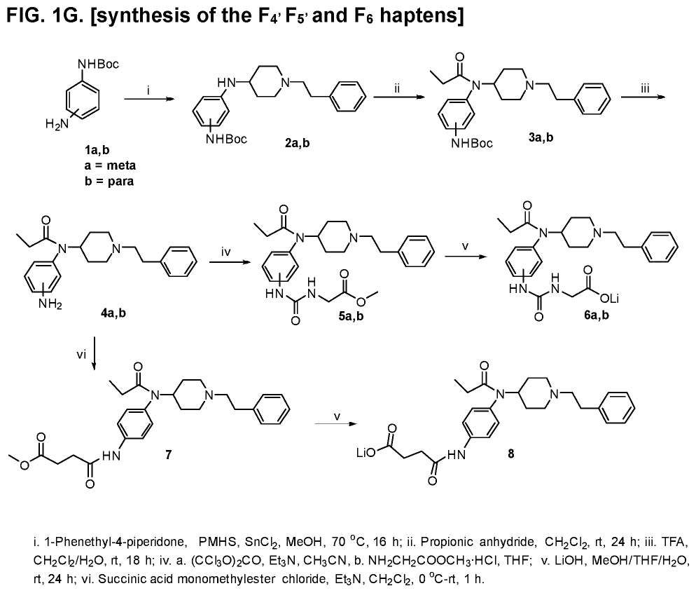

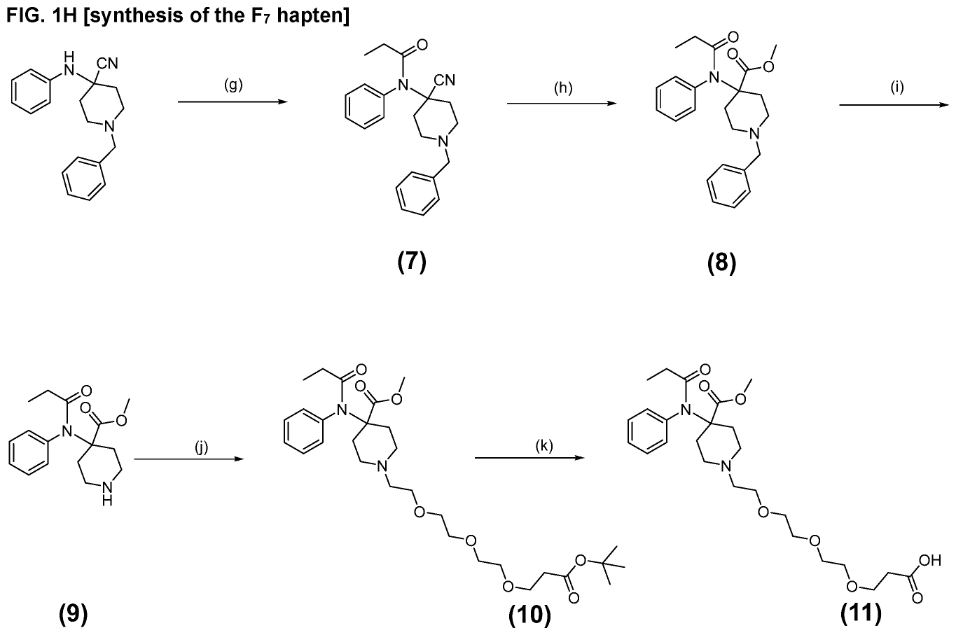

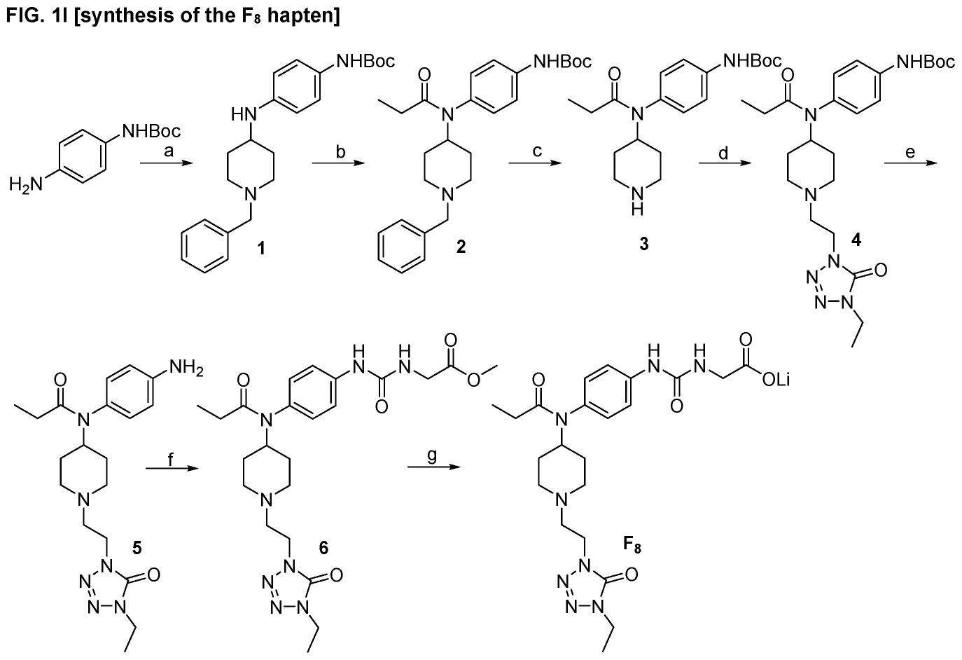

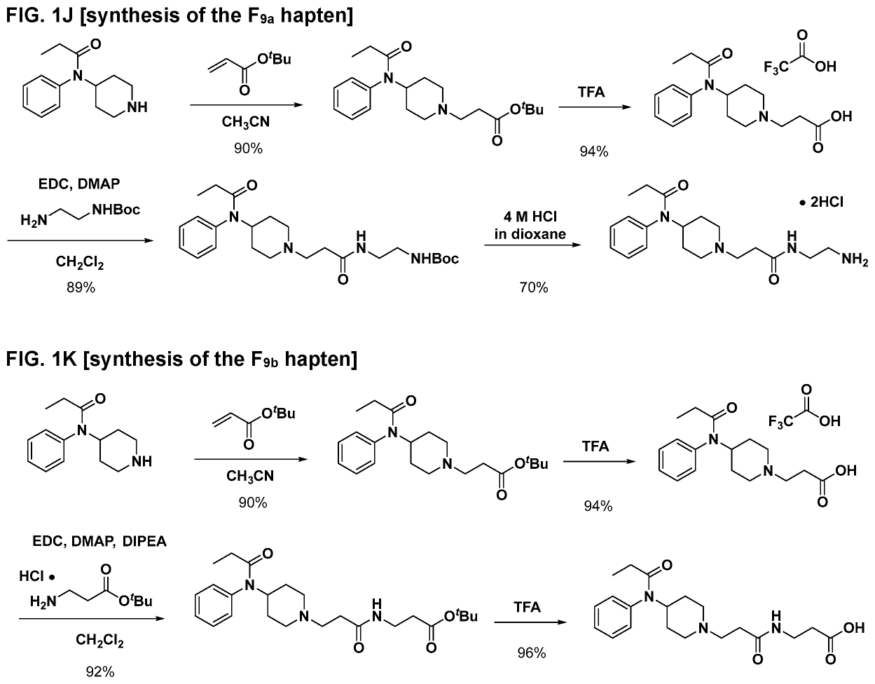

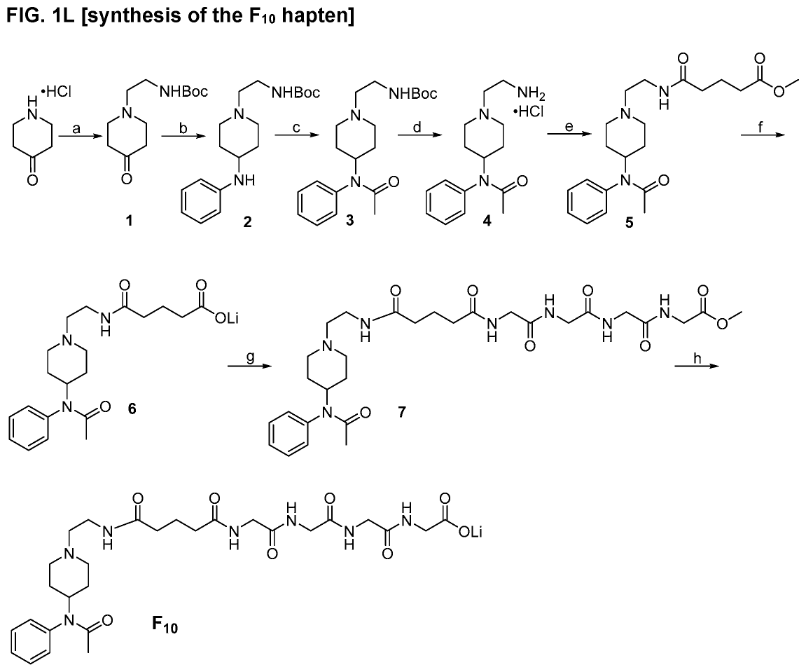

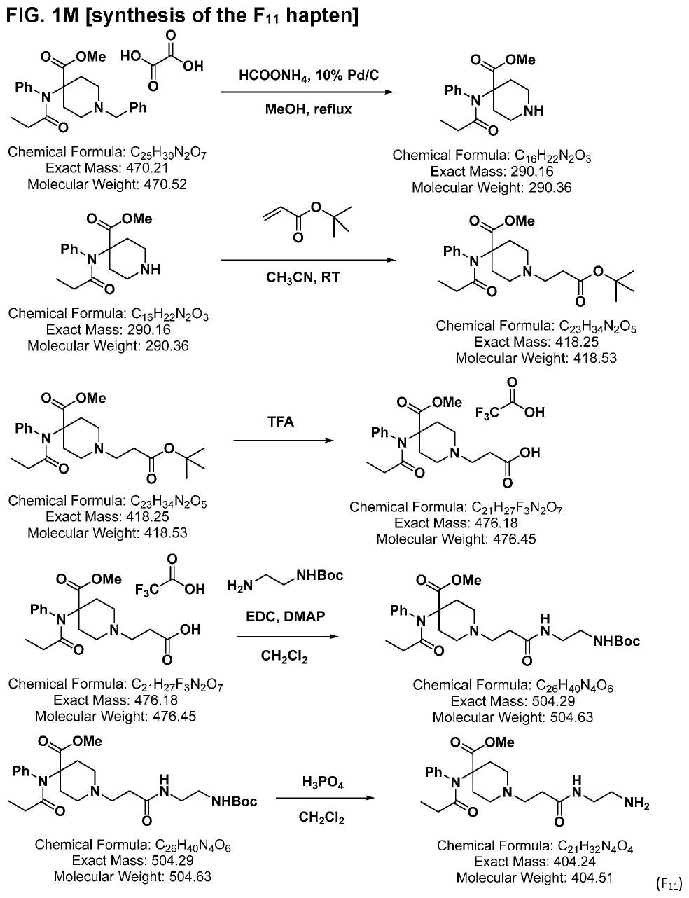

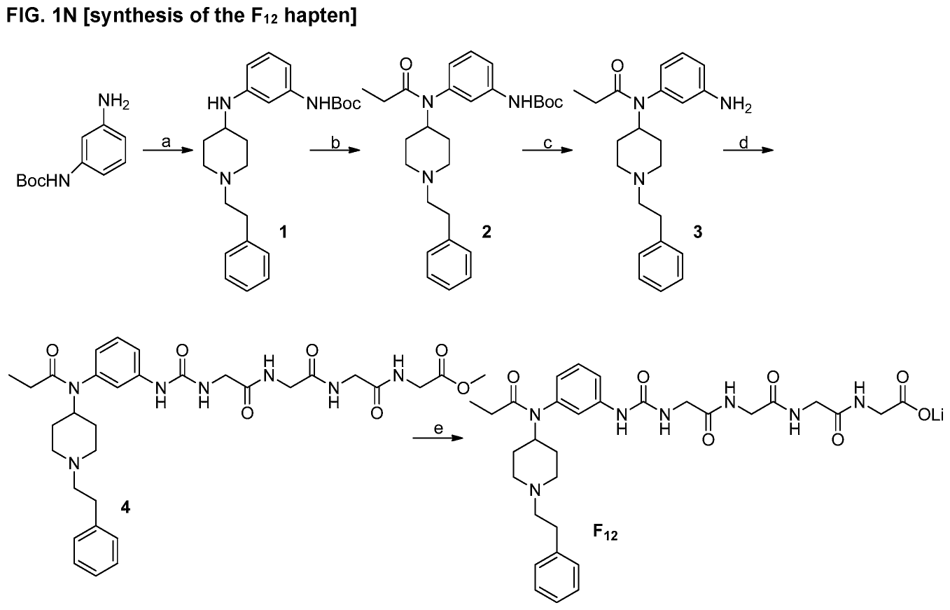

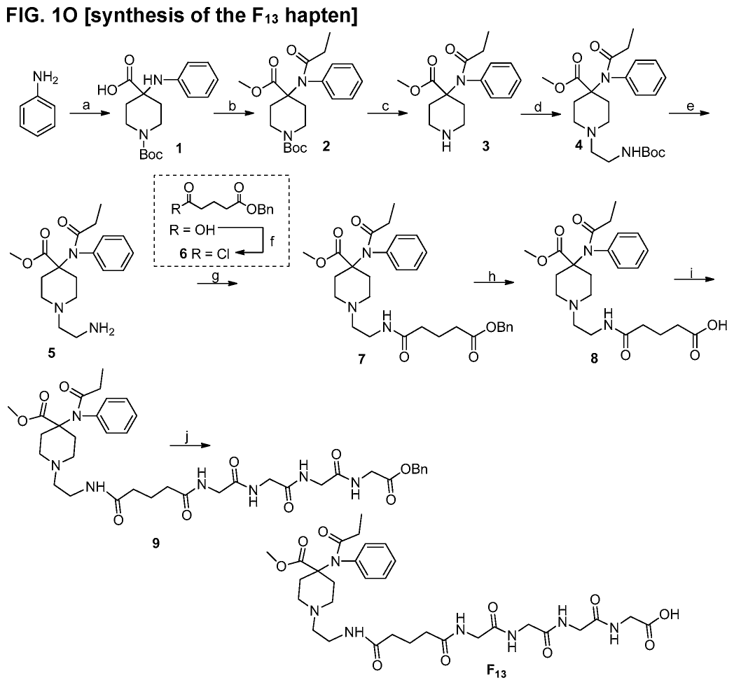

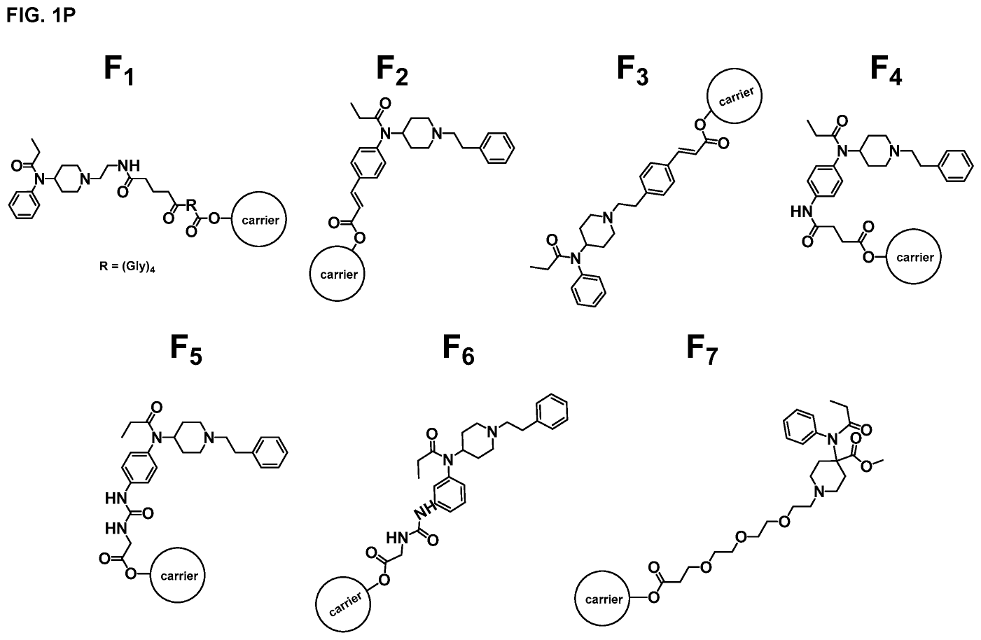

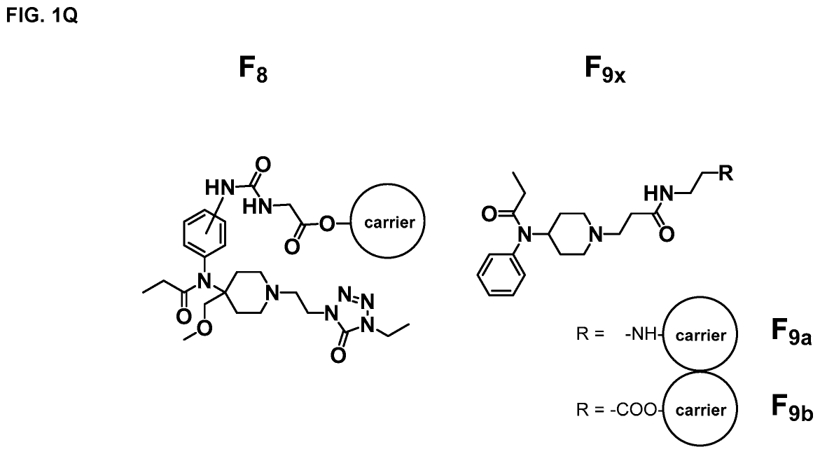

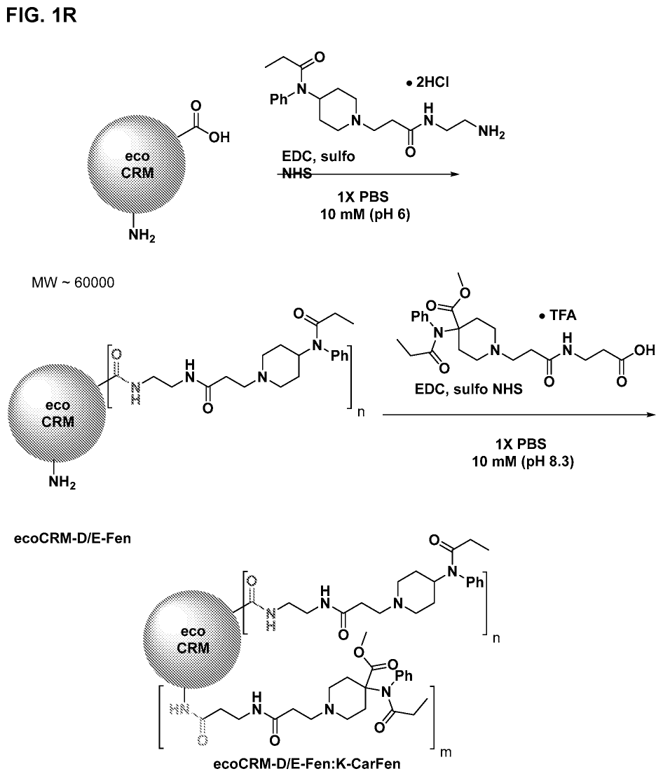

Fentanyl (FIG. 1A) is a schedule II opioid agonist with an extremely high in vivo potency 100-200 times than that of morphine. Fentanyl's analogs (including, for example, mefentanyl, α-mefentanyl, ohmefentanyl, phenaridine, carfentanil, lofentanil, sufentanil, alfentanil, brifentail, remifentanil, trefentanil, mirfenantil, alfentanil, acetylfentanyl, brorphine, a novel fentanyl analog, etc.) are either schedule II or schedule I opioids with even higher in vivo potency than fentanyl. Fentanyl has been increasingly used as an adulterant in heroin and counterfeit prescription opioids because of its potency, ease of chemical feasibility, and low manufacturing costs. Overdose deaths from heroin or hydrocodone/acetaminophen laced with fentanyl or its analogs have been increasingly reported in North America. The presence of fentanyl was also recorded in fatal overdoses related to cocaine, benzodiazepines, antidepressants, and other counterfeit or illicit drugs. In addition to fentanyl misuse in patients with opioid use disorders (OUDs), fentanyl and its derivatives have also been used as chemical agents for incapacitation in military scenarios. Finally, fentanyl and its analogs pose a potential risk for law enforcement officials, first responders, airport or custom personnel and their canine units.

The development of novel treatment modalities to reduce the incidence or severity of accidental overdoses from fentanyl and its derivatives could have substantial public health impact.

### Current Treatments for Fentanyl Overdose

The current treatment for fentanyl overdose is naloxone, an opioid antagonist. Distribution of naloxone to high-risk populations is being expanded in the US and has been shown to be a cost-effective strategy for decreasing overdose deaths in both the US and UK. However, for naloxone to be effective, administration is required shortly after exposure and with proper technique. Due to this consideration, as well as fentanyl's potency, naloxone may not always be sufficient to rapidly reverse fentanyl-induced respiratory depression. Also, administration of naloxone may precipitate opioid-withdrawal syndrome and cause severe side effects.

In contrast, prophylactic and therapeutic vaccination against fentanyl could be a cost-effective, long-lasting intervention to reduce prevalence of OUD and incidence or severity of fentanyl overdose. Moreover, if sufficiently selective, an anti-opioid vaccine could be combined with a current pharmacological treatment for OUD and/or overdose.

### Opioid Vaccines

Opioid vaccines have been explored pre-clinically as a treatment for OUD and have been effective in rodent and non-human primate models (Bremer et al. J Med Chem 55, 10776-10780 (2012), Bremer et al. J Am Chem Soc 139, 8601-8611 (2017), Bremer et al. Mol Pharm 11, 1075-1080 (2014), Matyas et al. Vaccine 31, 2804-2810 (2013), Pravetoni et al. Vaccine 30, 4617-4624 (2012), Raleigh et al. PLoS One 9, e115696 (2014), Raleigh et al. PLoS One 12, e0184876 (2017), Raleigh et al. J Pharmacol Exp Ther 344, 397-406 (2013), Schlosburg et al. Proc Natl Acad Sci USA 110, 9036-9041 (2013), Stowe et al. J Med Chem 54, 5195-5204 (2011)). Opioid vaccines elicit opioid-specific antibodies that selectively bind to the targeted opioids in the blood and reduce their distribution to the brain, reducing their behavioral and toxic effects. Vaccine efficacy is greatest when the levels of antibody produced are high and the opioid dose is low. Because fentanyl and its analogs are very potent and have a relatively low toxic dose compared to other abused opioid such as heroin or oxycodone, these compounds are particularly attractive candidates for this approach.

A limited number of studies showed pre-clinical proof of concept for immunotherapy against fentanyl and its analogs in mice, rats, rabbits, dogs, and non-human primates (Bremer et al. Angew Chem Int Ed Engl 55, 3772-3775 (2016), Hwang et al. ACS Chem Neurosci 9, 1269-1275 (2018), Raleigh et al. J Pharmacol Exp Ther 368, 282-291 (2019), Tenney et al. Neuropharmacology 158, 107730 (2019), Torten et al. Nature 253, 565-566 (1975), Townsend et al. Neuropsychopharmacology 44, 1681-1689 (2019), Robinson et al. J Med Chem 63, 14647-14667 (2020)). These studies showed that fentanyl-specific antibodies reduced hotplate antinociception and fentanyl-induced respiratory depression following small fentanyl doses. These studies suggest that fentanyl-based haptens induce antibodies that also cross react with fentanyl analogs such as alpha-methylfentanyl or cis-3-methylfentanyl (Bremer et al. Angew Chem Int Ed Engl 55, 3772-3775 (2016)). These studies involved either passive immunization with polyclonal antibodies or active vaccination using Freund's complete adjuvant intradermally (Torten et al. Nature 253, 565-566 (1975), Henderson et al. J Pharmacol Exp Ther 192, 489-496 (1975)) or other adjuvants administered i.p. (Bremer et al. Angew Chem Int Ed Engl 55, 3772-3775 (2016), Hwang et al. ACS Chem Neurosci 9, 1269-1275 (2018)), each of which may not be feasible in humans.

Although the use of active immunization with fentanyl haptens conjugated to carriers for blocking fentanyl analgesia has been previously suggested (see U.S. Publication No. 2014/0093525A1), the efficacy of any such vaccine and its ability to be used in combination with naloxone were unpredictable. Moreover, as further described in Example 1, the ability of a fentanyl hapten-carrier conjugate vaccine to reduce or prevent fentanyl-induced respiratory depression and to reduce or prevent bradycardia (cardiac toxicity) were unexpected, particularly because reduction of fentanyl-induced bradycardia is unprecedented. The reduction of fentanyl-induced bradycardia indicates a potential to counteract fatal overdoses because fentanyl has been associated with the Wooden Chest Syndrome (WCS) which includes a fentanyl-induced rigidity of the chest wall and upper airways, and which compromises the ability of conducting CPR in overdosing patients. While the mechanisms underlying WCS are not entirely understood, it appears that WCS is primarily mediated by alpha-adrenergic and cholinergic signaling rather than opioid receptor signaling (Torralva et al. J Pharmacol Exp Ther 371, 453-475 (2019)). Hence, naloxone is not effective in reversing or preventing WCS. Instead a vaccine that reduces the concentration of free (unbound) fentanyl, and reduces or prevents fentanyl-induced bradycardia may be particularly beneficial in limiting the occurrence of fentanyl-induced WCS.

As described in Example 1, studies were conducted to determine the efficacy of a series of vaccines including structurally diverse fentanyl haptens conjugated to immunogenic carrier proteins and adsorbed on aluminum adjuvant and administered intramuscularly (i.m.) in mice or rats.

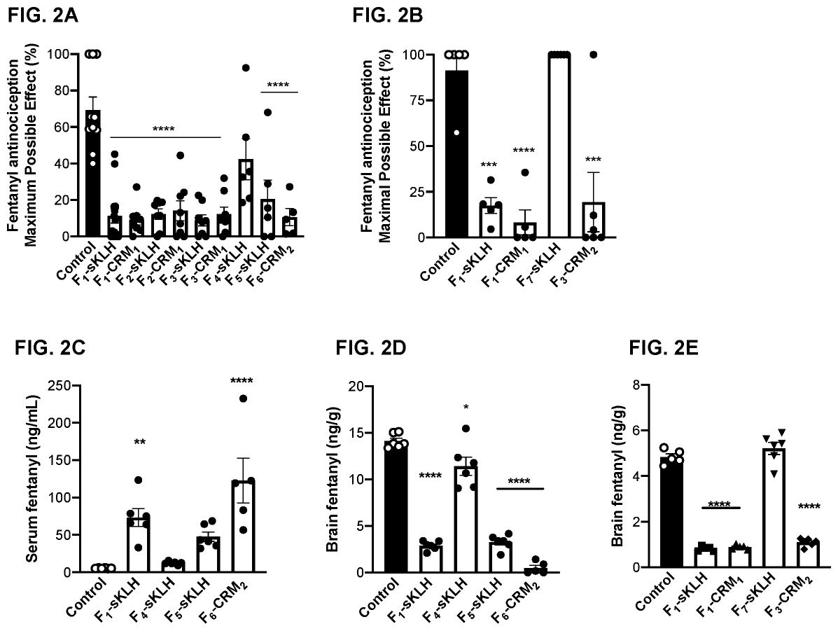

Mice and rats immunized with fentanyl hapten-carrier conjugates including haptens F₁, F₂, F₃, F₅, or F₆, had significantly (p≤0.0001 compared to control) lower fentanyl-induced antinociception compared to controls. (FIG. 2.) Mice and rats immunized with fentanyl hapten-carrier conjugates including haptens F₁, F₂, F₃, F₄, F₅, or F₆, had significantly (p≤0.05 compared to control) lower fentanyl-induced antinociception compared to controls. (FIG. 2.)

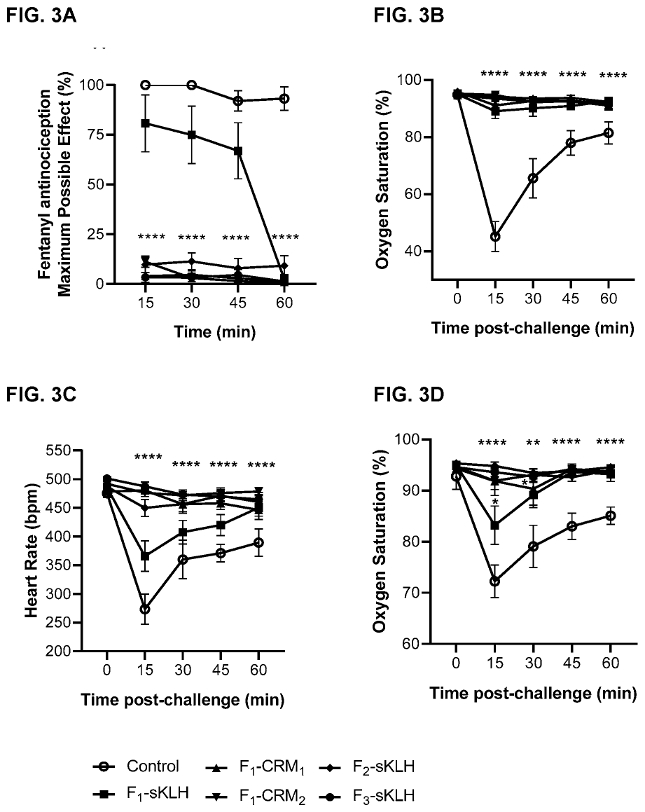

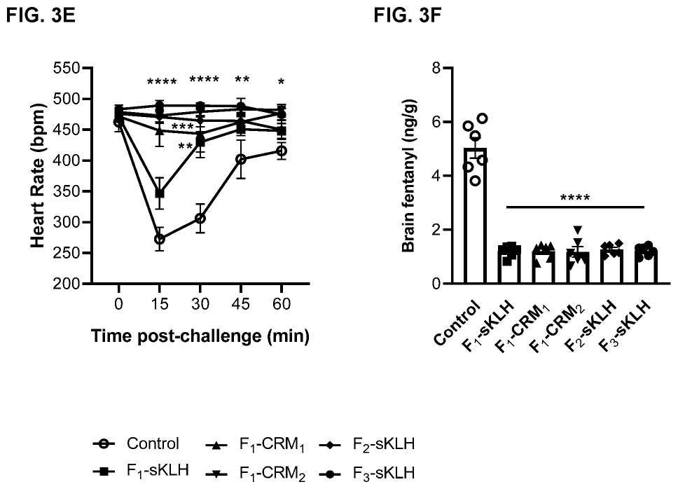

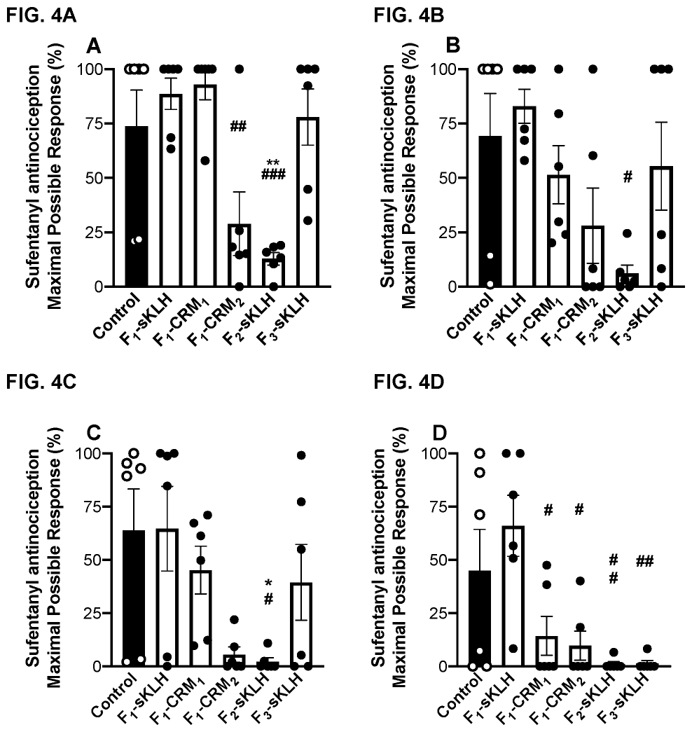

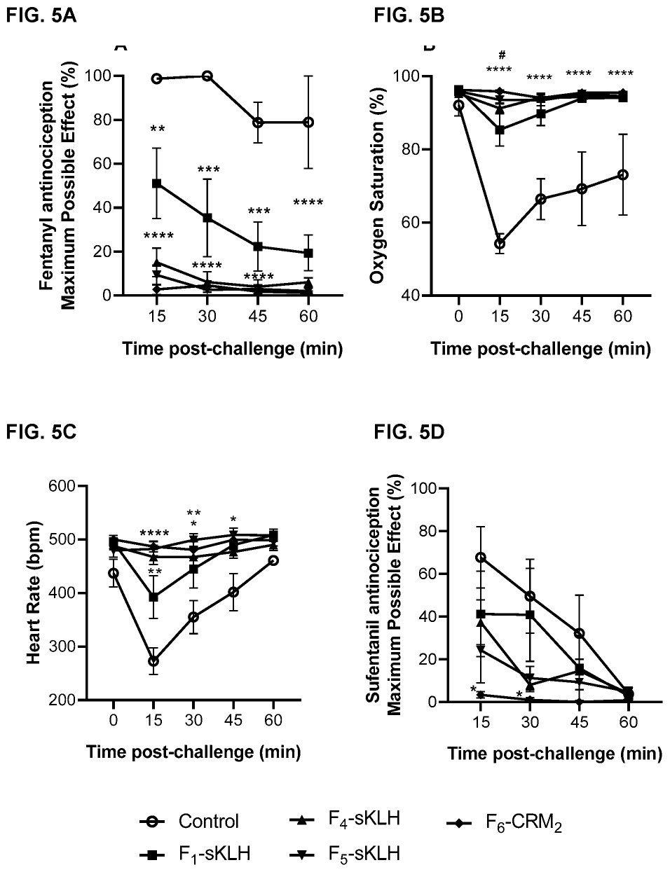

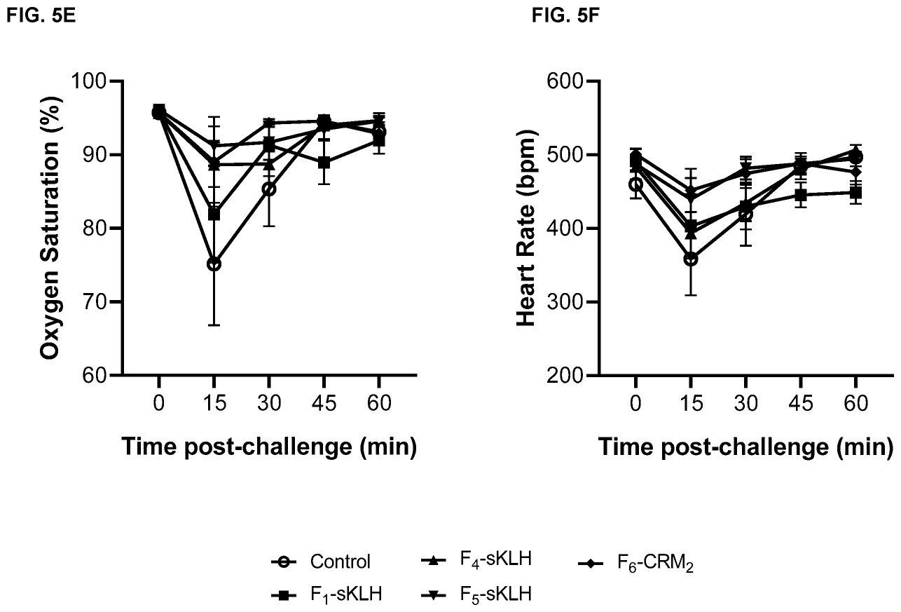

Fentanyl hapten-carrier conjugates including haptens F₁, F₂, or F₃ were effective in reducing fentanyl-induced respiratory depression (FIG. 3D), bradycardia (FIG. 3E), and distribution of fentanyl to the brain (FIG. 3F) in rats. Selected conjugates containing F₁, F₂ and F₃ haptens were effective in reducing sufentanil-induced antinociception in rats (FIG. 4). Similar results against fentanyl and sufentanil were obtained with fentanyl hapten-carrier conjugates including haptens F₄, F₅, or F₆ in rats (FIG. 5). Together, these data suggest that a fentanyl vaccine could be a viable option for reducing the respiratory depressive effects as well as cardiac toxicity of fentanyl (and its synthetic analogs) in humans, and possibly prevent or reduce the likelihood of fatal overdoses upon accidental or deliberate intake of fentanyl, fentanyl analogs, fentanyl-laced drug mixtures, and fentanyl analog-laced drug mixtures.

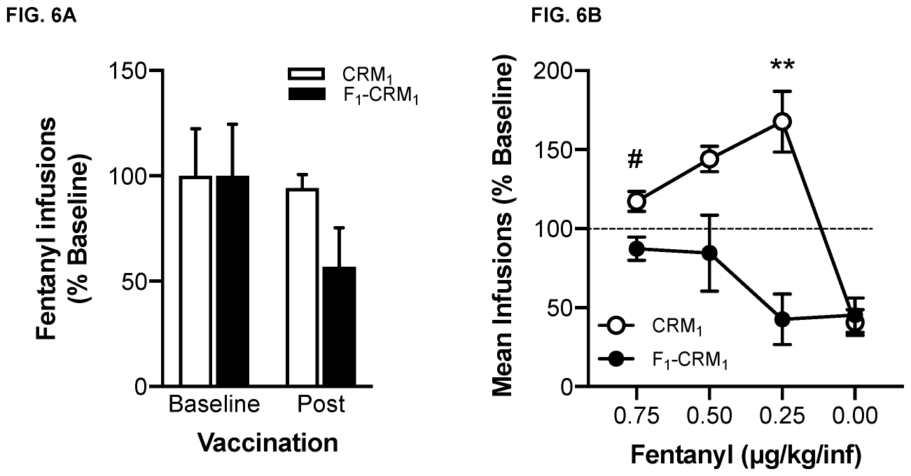

As shown in FIG. 6, vaccination reduced fentanyl intake in rats with ongoing fentanyl intravenous self-administration (FSA). These data suggest that ongoing opioid use does not affect the immunogenicity and efficacy of the vaccine, and that individuals vaccinated against fentanyl will likely not increase their fentanyl intake to overcome the effects of the vaccine. These data also suggest that, in contrast to opioid receptor antagonists such as naltrexone, an opioid detoxification regimen would not be required prior to initiation of immunotherapy with the vaccine in patients with ongoing use of fentanyl or another opioid.

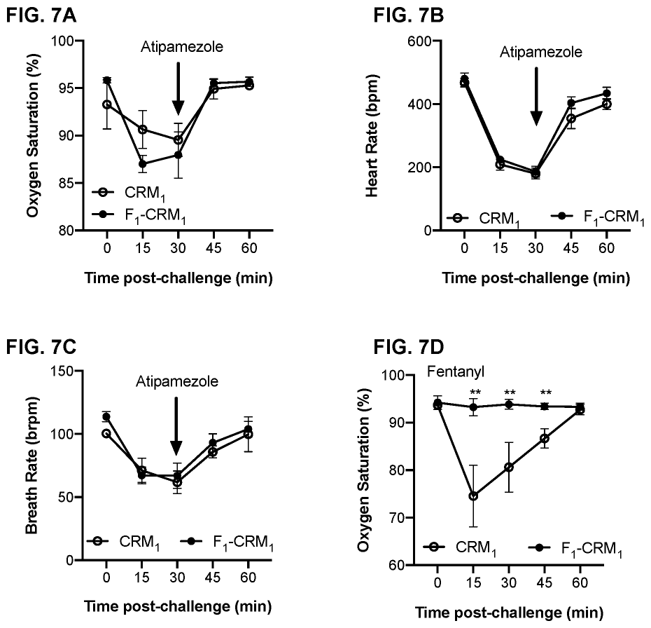

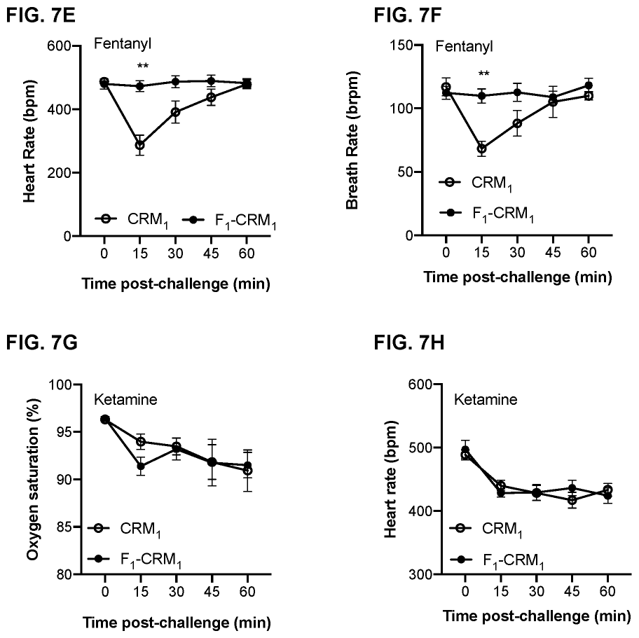

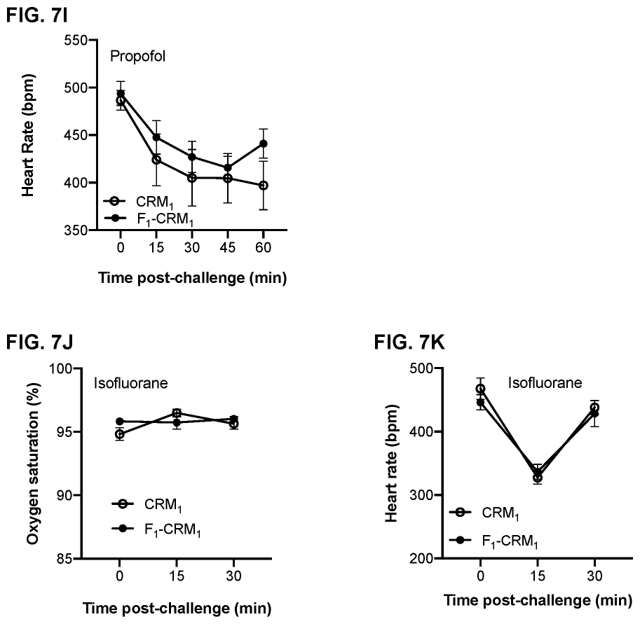

As shown in FIG. 7, vaccination selectively reduce fentanyl-induced respiratory depression while preserving induction of anesthesia with 2% inhaled isoflurane, 0.25 mg/kg dexmedetomidine (reversed by 1 mg/kg atipamezole), 75 mg/kg ketamine, and 100 mg/kg propofol. These studies suggest that individuals vaccinated against fentanyl can safely undergo anesthesia during surgical or routing invasive procedures using common pharmacological agents to both induce and reverse anesthesia. In addition, as show in Table 2C, vaccine-induced polyclonal antibodies did not bind off-target opioid-based medications such as buprenorphine, methadone, naloxone, and naltrexone, which are commonly used in treatment of opioid use disorders and prevention or reversal of opioid overdose.

### Fentanyl Haptens

In one aspect, this disclosure describes novel fentanyl haptens. The fentanyl hapten may include F₄, F₅, F₆, F₇, F₈, F₉ₐ, F_{9b}, F₁₀, F₁₁, F₁₂, or F₁₃, the structures of each of which are shown in FIG. 1B, or a combination thereof.

Fentanyl-based haptens F₄, F₅, F₆, F₇, F₈, F₉ₐ, F_{9b} and F₁₀ may be divided into three fentanyl derivative categories: (a) replacement of 2-ethyl-benzyl group with lysine reactive linking species (F₇, F₉ₐ, F_{9b}, F₁₀, F₁₁, F₁₃-F₇, F₁₁, F₁₃ are carfentanil based haptens), (b) modification of the 4-aminophenyl ring at the para position (F₄, F₅ and F₈-F₈ is an alfentanil based hapten), and (c) modification of the aminophenyl ring at the meta position (F₆ and F₁₂).

F₄, F₅, F₆, F₈ and F₁₂ haptens retain their respective fentanyl or alfentanil-core structure, including the N-phenyl moiety (FIG. 21A). Other previously published haptens (see, for example, FIG. 21C) retained the full structure of the target compound. F₁, F₉ₐ, F_{9b}, F₁₀, F₁₁ and F₁₃, however, lack the N-phenethyl moiety of the parent compound (fentanyl, acetylfentanyl and carfentanil) (FIG. 21). As further discussed herein, surprisingly, these haptens are still effective at raising an immune response to the parent compound. That is, this disclosure describes hapten structures that do not fully model the entire structure of the target compound but rather portions thereof and yet, surprisingly, exhibit the ability to act as vaccine components for the parent compounds.

When a combination of haptens is desired, any suitable combination may be selected. In some embodiments, it may be desired to select a combination of haptens based on different opioids.

Moreover, these haptens are readily prepared using straightforward synthetic approaches. Exemplary synthetic approaches for each hapten are described in the Examples.

### Fentanyl Hapten-Carrier Conjugate

In another aspect this disclosure describes a fentanyl hapten-carrier conjugate.

The fentanyl hapten-carrier conjugate comprises a fentanyl-based hapten F₁, F₂, F₃, F₄, F₅, F₆, F₇, F₈, F₉ₐ, F_{9b}, F₁₀, F₁₁, F₁₂, or F₁₃, or a combination thereof, the structures of each of which are shown in FIG. 1B-FIG. 1C. As used herein, “fentanyl hapten” refers to molecule which, when combined with a carrier, can elicit the production of antibodies which bind to fentanyl or its analogs. Exemplary conjugates of each F₂, F₃, F₄, F₅, F₆, F₇, F₈, F₉ₐ, and F_{9b}, are shown in FIG. 1P.

Unlike other natural or semi-synthetic opioids, fentanyl and its related synthetic opioids possess no intrinsic synthetic handles for conjugation to carrier proteins for the purposes of vaccine generation. To overcome this challenge, molecular analogs of the target opioid must be generated bearing substituents suitable for conjugation. Example 1 describes the creation of eight molecularly distinct lysine-reactive fentanyl analogs, varying the structure of linking groups and aryl ring substituent pattern in order to perform an unbiased hapten screen to generate vaccines with the greatest affinity towards fentanyl and related synthetic opioid analogs. Structural diversity of the hapten library was achieved through modification of fentanyl's structure to create haptens with different presentation to the immune system. Although the structure of the F₁ hapten has been previously reported (see U.S. Publication No. 2014/0093525), in this study the F₁ hapten was generated using an improved synthetic scheme that yielded a purer compound that facilitates conjugation to carriers.

Fentanyl-based haptens F₁, F₂, F₃, F₄, F₅, F₆, F₇, F₈, F₉ₐ, F_{9b}, F₁₀, F₁₁, F₁₂, or F₁₃, may be divided into four fentanyl derivative categories: (a) replacement of 2-ethyl-benzyl group with lysine reactive linking species (F₁, F₇, F₉ₐ, F_{9b}, F₁₀, F₁₁, F₁₃-F₇, F₁₁, F₁₃ are a carfentanil based haptens), (b) modification of the 4-aminophenyl ring at the para position (F₂, F₄, F₅ and F₈-F₈ is an alfentanil-based hapten), (c) modification of the aminophenyl ring at the meta position (F₆ and F₁₂), and (d) modification of the para position on the 2-ethyl-benzyl with an acrylic acid moiety (F₃).

These haptens are readily prepared using straightforward synthetic approaches and have a synthetic handle capable of ligation to carrier proteins.

When a combination of haptens is desired, any suitable combination may be selected. Some exemplary combinations include, for example, F₁ and F₁₃; F₁ and F₆; F₁ and F₉ₐ; F₁ and F_{9b}; F₁ and F₁₀; F₁ and F₅; F₃ and F₄; F₅, F₆, and F₇; F₁₀ and F₁₁; F₅ and F₆; F₅ and F₉ₐ; F₅ and F_{9b}; F₅ and F₁₀, F₅ and F₁₃; F₅, F₆, F₉ₐ, F_{9b}, F₁₀, and F₁₃, etc.

In some embodiments, it may be desired to select a combination of haptens based on different opioids. For example, a fentanyl-based hapten may be combined with a carfentanil-based hapten (including, for example, F₁ and F₁₃). In another example, a fentanyl-based hapten may be combined with a carfentanil-based hapten, an alfentanil-based hapten, or an acetylfentanyl-based hapten, or a combination thereof. In another example, a fentanyl-based hapten may be combined with a carfentanil-based hapten, an alfentanil-based hapten, and an acetylfentanyl-based hapten. Exemplary combinations include F₄ (a fentanyl-based hapten) and F₈ (an alfentanil-based hapten); F₉ₐ or F_{9b} (fentanyl-based haptens) and F₁₀ (an acetylfentanyl-based hapten); F₁ (a fentanyl-based hapten) and F₁₁ (a carfentanil-based hapten); F₁ (a fentanyl-based hapten), F₁₀ (an acetylfentanyl-based hapten), and F₁₃ (a carfentanil-based hapten). Other combinations are envisioned although they may not be specifically identified herein.

In some embodiments, it may be desired to select a combination of haptens based on different opioids, other than fentanyl-based analogs. For example, fentanyl-based or carfentanil-based haptens may be combined with an opioid-based hapten. The opioid based hapten may be designed to target heroin, 6-acetylmorphine, morphine, oxycodone, hydrocodone, and other derivatives of the morphinan structure. In some embodiments, it may be desired to select a combination of haptens based on different substance of abuse, rather than opioid-based analogs. For example, fentanyl-based or opioid-based haptens may be combined with drug-based haptens designed to target a stimulant. Exemplary stimulants include nicotine, cocaine, methamphetamine, amphetamine, etc.

In the fentanyl hapten-carrier conjugate, the fentanyl hapten is conjugated to an immunogenic carrier.

Any suitable carrier may be used. In some embodiments, the immunogenic carrier includes, for example, bovine serum albumin (BSA), ovalbumin (OVA), keyhole limpet hemocyanin (KLH) including, for example, GMP grade subunit KLH (sKLH); diphtheria toxin; CRM (also referred to as CRM₁₉₇), a genetically detoxified form of diphtheria toxin including for example, E. coli-expressed CRM (EcoCRM) (Fina Biosolutions, Rockville, Md.) (also referred to herein as CRM₁) or CRM₁₉₇ (PFEnex, San Diego, Calif.) (also referred to herein as CRM₂); tetanus toxin or tetanus toxoid (TT); Pseudomonas exotoxin A; cholera toxin or toxoid; a Group A streptococcal toxin; a liposome; human gamma globulin; chicken immunoglobulin G; bovine gamma globulin; pneumolysin of Streptococcus pneumoniae; filamentous haemagluttinin (FHA); FHA fragments of Bordetella pertussis; pili or pilins of Neisseria gonorrhoeae; pili or pilins of Neisseria meningitidis; outer membrane proteins of Neisseria meningitidis, outer membrane proteins of Neisseria gonorrhoeae; C5A peptidase of Streptococcus; a surface protein of Moraxella catarrhalis; macro-, micro-, and nano-particles or combinations thereof (including synthetic macro-, micro-, and nano-particles); a carbon-based particle; a nanocarrier; a protein or peptide of viral, bacterial, or synthetic origin; or another immunogenic component; or a mixture or combination thereof.

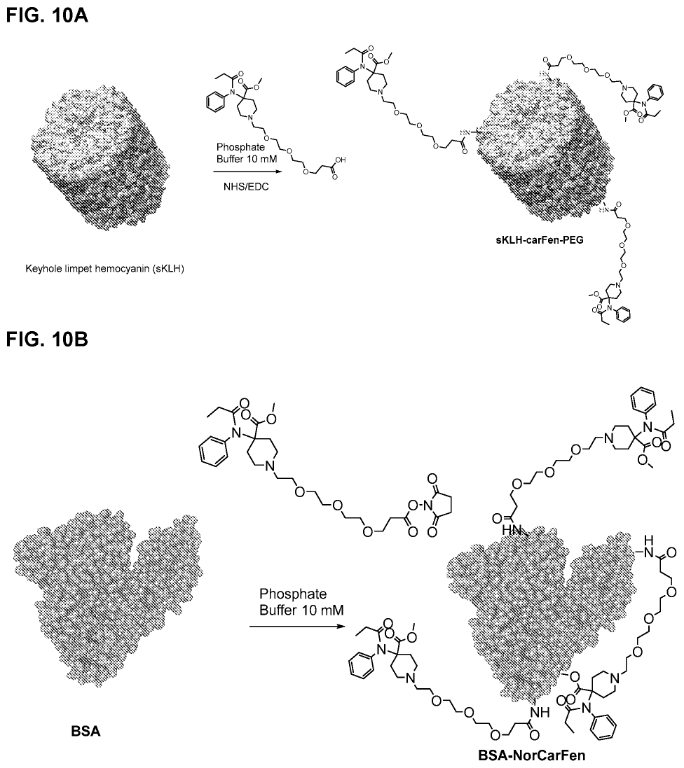

For example, a visual representation of the binding of the conjugation of F₇ to sKLH is shown in FIG. 10A. FIG. 10B shows a visual representation of the conjugation of F₇ and BSA.

The fentanyl hapten may be conjugated to an immunogenic carrier via any suitable coupling chemistry. In some embodiments, the fentanyl hapten may be conjugated to an immunogenic carrier via carbodiimide, maleimide, N-Hydroxy succinimide (NHS)-ester, or other coupling chemistry. For example, as further described in Example 1, upon completion and characterization of the hapten library, F₁, F₂, F₃, F₄, F₅, F₆, and F₇, were conjugated to lysine residues of EcoCRM, CRM₁₉₇, and sKLH using either in situ activation of a hapten's carboxylic acid groups (F₁, F₄, F₅, F₇, F₈, F_{9b}) or amino groups (F₉ₐ), or conjugation using N-Hydroxy succinimide-activated hapten esters (F₂, F₃). The hapten-protein conjugates were purified using size exclusion-based filtration methods (either dialysis or centrifuge filtration) and subsequently characterized using MALDI-ToF to characterize total number of haptens per protein or DLS for size.

As further described in Example 5, haptens of an expanded hapten library, further including F₁₀, F₁₁, F₁₂, and F₁₃, were also conjugated to carriers. F₁₀, F₁₂, and F₁₃ were reacted to lysine residues via an in situ EDC activated ester (activation of a hapten's carboxylic acid groups). F₁₁ was conjugated to aspartic and glutamic acid residues via an in situ EDC activated ester (using N-Hydroxy succinimide-activated hapten esters).

Without wishing to be bound by theory, it is believed that conjugation of a hapten to the immunogenic carrier via a carboxylic acid group, an amino group, or hydroxysuccinimide (see FIG. 1P) may better maintain the hapten in a natural configuration than conjugation via a propionamide group, promoting hapten stability and resulting in better vaccine efficacy.

In some embodiments, multiple fentanyl haptens may be conjugated to a single immunogenic carrier. In some embodiments, multiples of the same fentanyl hapten may be conjugated to a single immunogenic carrier. In some embodiments, different fentanyl haptens may be conjugated to a single immunogenic carrier (see FIG. 1R). BSA is typically used to optimize the conjugation reaction. In some embodiments the protein haptenation ratio (number of hapten molecules per carrier molecule) is measured with mass spectrometry. In some embodiments, a higher haptenization ratio may enhance immunogenicity of the fentanyl hapten-carrier conjugate. In some embodiments, the number of hapten molecules per carrier molecule is at least 1. In some embodiments, the number of hapten molecules per carrier molecule is at least 5, at least 10, at least 15, at least 20, at least 30, at least 40, or at least 50. In some embodiments, the number of hapten molecules per carrier molecule is up to 30, up to 40, or up to 50, up to 100, up to 200, or up to 300. In an exemplary embodiment, the number of hapten molecules per KLH is in a range of 50 to 300. In another exemplary embodiment, the number of hapten molecules per KLH is in a range of 50 to 100. In yet another exemplary embodiment, the number of hapten molecules per KLH is in a range of 100 to 200. In a further exemplary embodiment, the number of hapten molecules per KLH is in a range of 200 to 300.

Exemplary haptenization ratios for the haptens described herein are shown in Table 1A-Table 1B.

In some embodiments, a fentanyl hapten may be conjugated to a single immunogenic carrier along with other structurally distinct or structurally-related fentanyl-derived haptens to provide a multivalent display targeting multiple fentanyl analogs at once. In some embodiments, a fentanyl hapten-carrier conjugate can be co-administered with other fentanyl hapten-carrier conjugates to provide a multivalent immunization strategy targeting multiple fentanyl analogs at once. In some embodiments, a fentanyl hapten-carrier conjugate can be co-administered with other opioid hapten-carrier conjugates to provide a multivalent immunization strategy targeting multiple opioids at once (for example, fentanyl and heroin). In some embodiments, a fentanyl hapten-carrier conjugate can be co-administered with other drug hapten-carrier conjugates to provide a multivalent immunization strategy targeting multiple drugs at once (e.g., fentanyl and cocaine, or fentanyl and methamphetamine).

In an exemplary embodiment, a fentanyl hapten-carrier conjugate may include two or more different haptens, wherein each fentanyl hapten is conjugated to a separate one of two or more immunogenic carriers. In some embodiments, at least one of the haptens is selected from F₁, F₂, F₃, F₄, F₅, F₆, F₇, F₈, F₉ₐ, F_{9b}, F₁₀, F₁₁, F₁₂, and F₁₃. In some embodiments, each of the two or more different haptens is selected from F₁, F₂, F₃, F₄, F₅, F₆, F₇, F₈, F₉ₐ, F_{9b}, F₁₀, F₁₁, F₁₂, and F₁₃. The immunogenic carriers may be the same immunogenic carrier or different immunogenic carriers. For example, the fentanyl hapten-carrier conjugate may include F₁ and F₅, where F₁ is conjugated to CRM and F₅ is conjugated to sKLH. In another example, the fentanyl hapten-carrier conjugate may include F₄ and F₅, where F₄ is conjugated to one sKLH molecule and F₅ is conjugated to a different sKLH molecule.

In another exemplary embodiment, the fentanyl hapten-carrier conjugate may include two or more different haptens, wherein each fentanyl hapten is conjugated to a single immunogenic carrier. In some embodiments, at least one of the haptens is selected from F₁, F₂, F₃, F₄, F₅, F₆, F₇, F₈, F₉ₐ, F_{9b}, F₁₀, F₁₁, F₁₂, and F₁₃. In some embodiments, the two or more different haptens are selected from F₁, F₂, F₃, F₄, F₅, F₆, F₇, F₈, F₉ₐ, F_{9b}, F₁₀, F₁₁, F₁₂, and F₁₃. For example, the fentanyl hapten-carrier conjugate may include F₃ and F₆, where both F₃ and F₆ are conjugated to the same CRM molecule. In another example, as shown in FIG. 1R, both a fentanyl-based hapten and a carfentanil-based hapten are conjugated to the same carrier protein (CRM is shown in FIG. 1R as an exemplary carrier protein).

In some embodiments, the haptens may include a —COOH group or an amino group to facilitate labeling. In some embodiments, including when two or more different haptens are used, one hapten may include a —COOH group and the other hapten may include an amino group.

In yet another exemplary embodiment, the fentanyl hapten-carrier conjugate (including F₁, F₂, F₃, F₄, F₅, F₆, F₇, F₈, F₉ₐ, F_{9b}, F₁₀, F₁₁, F₁₂, or F₁₃, or combinations thereof) may be administered in a multivalent formulation alongside with a vaccine targeting other opioids (including, for example, heroin or oxycodone) or other drugs of abuse (including, for example, cocaine, methamphetamine). Such a multivalent formulation could be used to treat or prevent substance use disorders and toxicity related to mixtures (including, for example, heroin/fentanyl, cocaine/fentanyl, etc.).

### Compositions Including a Fentanyl Hapten

In some embodiments, this disclosure describes a composition including a fentanyl hapten-carrier conjugate described herein.

In some embodiments, the composition comprising the fentanyl hapten-carrier conjugate may further include an adjuvant or other delivery platform to augment the immunogenicity of the conjugate (for example, a particle, a bead, etc.). Any suitable adjuvant or delivery platform may be included. Exemplary adjuvants include, for example, an aluminum adjuvant or aluminum-based adjuvant (including, for example, aluminum salts such as aluminum potassium sulfate (alum), aluminum hydroxide, and aluminum phosphate), complete Freund's adjuvant (CFA), incomplete Freund's adjuvant (IFA), a phytol-based adjuvant, a toll like receptor (TLR) agonist (including, for example, monophosphoryl lipid A (MPLA) or another TLR ligand-based adjuvant), a oligomerization domain (NOD)-like receptor (NLR) agonist, a RIG-I-like receptor (RLR) agonist, a C-type lectin receptor (CLR) agonist, degradable nanoparticles (including, for example, poly-lactid-co-glycolid acid (PLGA)), or non-degradable nanoparticles (including, for example, latex, gold, silica or polystyrene), or combinations thereof.

Additionally or alternatively, suitable adjuvants include but are not limited to surfactants, for example, hexadecylamine, octadecylamine, lysolecithin, dimethyldioctadecylammonium bromide, N,N-dioctadecyl-N′—N-bis(2-hydroxyethyl-propane di-amine), methoxyhexadecyl-glycerol, and pluronic polyols; polyanions, for example, pyran, dextran sulfate, poly IC, polyacrylic acid, carbopol; peptides, for example, muramyl dipeptide, dimethylglycine, tuftsin, oil emulsions, alum, and mixtures thereof. Other potential adjuvants include the B peptide subunits of E. coli heat labile toxin or of the cholera toxin, or CpG oligonucleotides.

In some embodiments, the adjuvant may preferably include aluminum or an aluminum-based adjuvant (including, for example, aluminum salts such as aluminum potassium sulfate (alum), aluminum hydroxide, and aluminum phosphate). As further described in Example 1, in an exemplary embodiment, fentanyl hapten-carrier conjugates were formulated in aluminum adjuvant and tested for efficacy in mice and rats.

In some embodiments, the composition comprising the fentanyl hapten-carrier conjugate and an adjuvant may include the fentanyl hapten-carrier conjugate adsorbed on the adjuvant. For example, in an exemplary embodiment, the fentanyl hapten may be adsorbed on an aluminum-based adjuvant (including, for example, aluminum salts such as aluminum potassium sulfate (alum), aluminum hydroxide, and aluminum phosphate).

In some embodiments, the composition comprising the fentanyl hapten-carrier conjugate may preferably include a toll like receptor (TLR) agonist or ligand.

In an exemplary embodiment, the composition comprising the fentanyl hapten-carrier conjugate includes a toll like receptor (TLR) agonist or ligand and the fentanyl hapten-carrier conjugate adsorbed on an aluminum-based adjuvant (including, for example, aluminum salts such as aluminum potassium sulfate (alum), aluminum hydroxide, and aluminum phosphate).

In some embodiments, the composition may include a particular ratio of the fentanyl hapten-carrier conjugate to adjuvant. For example, in some embodiments, the fentanyl hapten-carrier conjugate:adjuvant ratio may be at least 1:3 or at least 2:3. In some embodiments, the fentanyl hapten-carrier conjugate:adjuvant ratio may be up to 2:3 or up to 3:3 (1:1).

In some embodiments, the composition may also include, for example, buffering agents to help to maintain the pH in an acceptable range or preservatives to retard microbial growth. A composition may also include, for example, pharmaceutically acceptable carriers, excipients, stabilizers, chelators, salts, or antimicrobial agents. Acceptable pharmaceutically acceptable carriers, excipients, stabilizers, chelators, salts, preservatives, buffering agents, or antimicrobial agents, include, but are not limited to, buffers such as phosphate, citrate, and other organic acids; antioxidants including ascorbic acid and methionine; preservatives, such as sodium azide, octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol; polypeptides; proteins, such as serum albumin, gelatin, or non-specific immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (for example, Zinc (Zn)-protein complexes); and/or non-ionic surfactants such as TWEEN, PLURONICS, or polyethylene glycol (PEG).

In some embodiments, the composition is a pharmaceutical composition and includes the fentanyl hapten-carrier conjugate and a pharmaceutically acceptable carrier, diluent or excipient. In the preparation of the pharmaceutical compositions comprising the fentanyl hapten-carrier conjugate described herein, a variety of vehicles and excipients may be used, as will be apparent to the skilled artisan.

The pharmaceutical compositions will generally comprise a pharmaceutically acceptable carrier and a pharmacologically effective amount of the fentanyl hapten-carrier conjugate, or mixture of fentanyl hapten-carrier conjugates.

The pharmaceutical composition may be formulated as a powder, a granule, a solution, a suspension, an aerosol, a solid, a pill, a tablet, a capsule, a gel, a topical cream, a suppository, a transdermal patch, and/or another formulation known in the art.

For the purposes described herein, pharmaceutically acceptable salts of a fentanyl hapten-carrier conjugate are intended to include any art-recognized pharmaceutically acceptable salts including organic and inorganic acids and/or bases. Examples of salts include but are not limited to sodium, potassium, lithium, ammonium, calcium, as well as primary, secondary, and tertiary amines, esters of lower hydrocarbons, such as methyl, ethyl, and propyl. Other salts include but are not limited to organic acids, such as acetic acid, propionic acid, pyruvic acid, maleic acid, succinic acid, tartaric acid, citric acid, benzoic acid, cinnamic acid, salicylic acid, etc.

As used herein, “pharmaceutically acceptable carrier” comprises any standard pharmaceutically accepted carriers known to those of ordinary skill in the art in formulating pharmaceutical compositions. For example, the fentanyl hapten-carrier conjugate may be prepared as a formulation in a pharmaceutically acceptable diluent, including for example, saline, phosphate buffer saline (PBS), aqueous ethanol, or solutions of glucose, mannitol, dextran, propylene glycol, oils (for example, vegetable oils, animal oils, synthetic oils, etc.), microcrystalline cellulose, carboxymethyl cellulose, hydroxylpropyl methyl cellulose, magnesium stearate, calcium phosphate, gelatin, polysorbate 80 or as a solid formulation in an appropriate excipient.

A pharmaceutical composition will often further comprise one or more buffers (for example, neutral buffered saline or phosphate buffered saline), carbohydrates (for example, glucose, sucrose or dextrans), mannitol, proteins, polypeptides or amino acids such as glycine, antioxidants (for example, ascorbic acid, sodium metabisulfite, butylated hydroxytoluene, butylated hydroxyanisole, etc.), bacteriostats, chelating agents such as EDTA or glutathione, adjuvants (for example, aluminum hydroxide), solutes that render the formulation isotonic, hypotonic or weakly hypertonic with the blood of a recipient, suspending agents, thickening agents and/or preservatives. Alternatively, compositions of the present disclosure may be formulated as a lyophilizate.

Any suitable carrier known to those of ordinary skill in the art may be employed in a composition including at least fentanyl hapten-carrier conjugate describes herein. Compositions including a fentanyl hapten-carrier conjugate may be formulated for any appropriate manner of administration, including for example, oral, nasal, mucosal, intravenous, intraperitoneal, intradermal, subcutaneous, and intramuscular administration.

### Methods of Making the Fentanyl Hapten-Carrier Conjugate and Composition Including the Fentanyl Hapten-Carrier Conjugate

The hapten of the fentanyl hapten-carrier conjugate may be synthesized by any suitable means.

In some embodiments, F₁ of the fentanyl hapten-carrier conjugate may be synthesized as described in FIG. 1D and Example 1.

In some embodiments, F₂ of the fentanyl hapten-carrier conjugate may be synthesized as described in FIG. 1E and Example 1.

In some embodiments, F₃ of the fentanyl hapten-carrier conjugate may be synthesized as described in FIG. 1F and Example 1.

In some embodiments, F₄ of the fentanyl hapten-carrier conjugate may be synthesized as described in FIG. 1G and Example 1.

In some embodiments, F₅ of the fentanyl hapten-carrier conjugate may be synthesized as described in FIG. 1G and Example 1.

In some embodiments, F₆ of the fentanyl hapten-carrier conjugate may be synthesized as described in FIG. 1G and Example 1.

In some embodiments, F₇ of the fentanyl hapten-carrier conjugate may be synthesized as described in FIG. 1H and Example 1.

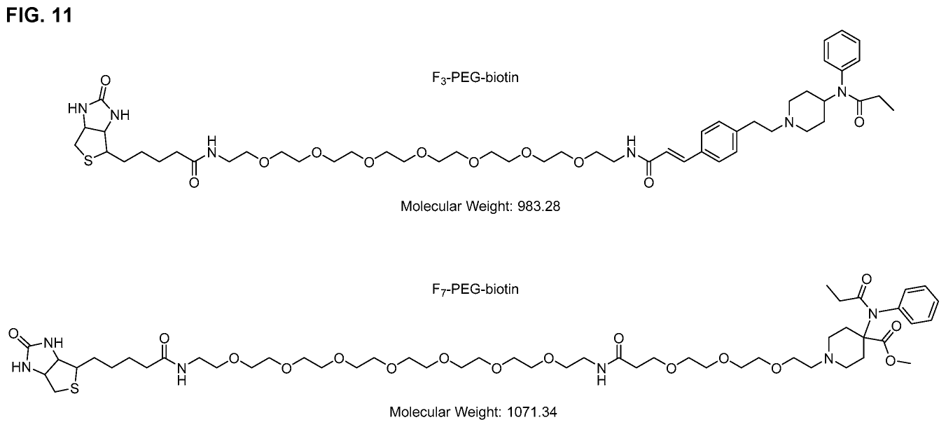

In some embodiments, F₈ of the fentanyl hapten-carrier conjugate may be synthesized as described in FIG. 11 and Example 2.

In some embodiments, F₉ₐ of the fentanyl hapten-carrier conjugate may be synthesized as described in FIG. 1J and Example 3.

In some embodiments, F_{9b} of the fentanyl hapten-carrier conjugate may be synthesized as described in FIG. 1K and Example 3.

In some embodiments, F₁₀ of the fentanyl hapten-carrier conjugate may be synthesized as described in FIG. 1L and Example 4.

In some embodiments, F₁₁ of the fentanyl hapten-carrier conjugate may be synthesized as described in FIG. 1M and Example 4.

In some embodiments, F₁₂ of the fentanyl hapten-carrier conjugate may be synthesized as described in FIG. 1N and Example 4.

In some embodiments, F₁₃ of the fentanyl hapten-carrier conjugate may be synthesized as described in FIG. 1O and Example 4.

In some embodiments, conjugation of two structurally different fentanyl haptens may be conjugated to the carrier as described in FIG. 1R.

In some embodiments, the identity and purity of the final product may be verified by mass spectrometry and/or NMR.

The fentanyl hapten may be conjugated to the carrier through any suitable means. In some embodiments, the fentanyl hapten may be conjugated to the carrier through coupling chemistry including, for example, through carbodiimide, maleimide, or NHS-ester chemistry. (See (Pravetoni et al. Vaccine 30, 4617-4624 (2012), Baruffaldi et al. Mol Pharm 15, 4947-4962 (2018), Baruffaldi et al. Mol Pharm 16, 2364-2375 (2019), Pravetoni et al. J Pharmacol Exp Ther 341, 225-232 (2012)); & Example 1.)

In an exemplary embodiment, conjugation of the hapten via carbodiimide (EDAC) chemistry may be performed as follows: the hapten may be dissolved in a buffer and activated by carbodiimide coupling chemistry using N-ethyl-N′-(3 dimethylaminopropyl) carbodiimide hydrochloride (EDAC, Sigma-Aldrich, St. Louis, Mo.) cross-linking. The mixture may be reacted at room temperature (RT) for at least 5 minutes and up to 12 hours. A carrier (for example, BSA, sKLH, or CRM) is added, and the reactions allowed to progress until conjugates are formed. In another exemplary embodiment, conjugation of the hapten via carbodiimide (EDAC) chemistry may be performed as described in Example 1.

In an exemplary embodiment, conjugation of the hapten via N-Hydroxy succinimide (NHS)-ester chemistry may be performed as follows: the hapten may be dissolved in in a buffer and added (for example, at a rate of 20 μL per minute) to a carrier (for example, BSA, sKLH, or CRM). The mixture may be reacted for at least 1 hour and up to 2 days at a temperature in a range of 4° C. to 37° C. The resulting conjugate may be diluted with buffer and/or purified using dialysis. In some exemplary embodiments, conjugation of the hapten via N-Hydroxy succinimide (NHS)-ester chemistry may be performed as described in Example 1.

The final conjugates may be ultrafiltered (for example, using Amicon filters), with the filter size (for example, 50 kDa or 100 kDa molecular cutoff) depending on the carrier protein dimensions. Additionally or alternatively, the final conjugates may be purified using Tangential Flow Filtration (TFF) including, for example, if reaction volumes are greater than 100 mL.

The resulting solutions may be stored at 4° C.

In some embodiments, to avoid precipitation or aggregation, including, for example, when CRM is used as the carrier, a sugar (or other suitable carbohydrate) may be included in the reacting buffer or storage buffer or both as a stabilizing agent. In an exemplary embodiment, the reacting buffer or storage buffer or both may include 250 mM sugar. In some embodiments, the sugar includes sucrose.

### Methods of Using the Fentanyl Hapten-Carrier Conjugate

In another aspect, this disclosure describes a method of using a fentanyl hapten-carrier conjugate or a composition including the fentanyl hapten-carrier conjugate. In some embodiments, the fentanyl hapten-carrier conjugate or a composition including the fentanyl hapten-carrier conjugate may be used in an anti-opioid vaccine. In some embodiments, the fentanyl hapten-carrier conjugate or a composition including the fentanyl hapten-carrier conjugate may be used as a prophylactic or therapeutic vaccine to counteract toxicity from exposure to fentanyl and its analogs and for the prevention and treatment of opioid use disorders. In some embodiments, the fentanyl hapten-carrier conjugate or a composition including the fentanyl hapten-carrier conjugate may be used as a prophylactic or therapeutic vaccine to counteract toxicity from exposure to opioids and other drugs of abuse and/or for the prevention and treatment of substance use disorders.

In some embodiments, the fentanyl hapten-carrier conjugate or a composition including the fentanyl hapten-carrier conjugate may be used to detect and/or purify antibodies or B cell lymphocytes specific for fentanyl and its analogs. Specifically, as described in Example 1, biotinylated analogs of fentanyl-based haptens may be used to generate reagents for immunoassays, detection, or diagnostics.

For example, FIG. 11 shows the conjugation of F₃ and F₇ to PEG and biotin. These or similar reagents can be attached to streptavidin or streptavidin conjugated to pychoerytrin and used as baits to sort or analyze opioid-specific B cells as described in Taylor et al. J. Immunol. Methods, 2014; 405:74-86 or Laudenbach et al., J. Immunol. 2015; 194(12):5926-36. These reagents can additionally or alternatively be used to isolate and analyze B cells specific for fentanyl and its analogs in mouse, rat, human, and other species of pre-clinical and clinical interest.

In some embodiments, a fentanyl hapten-carrier conjugate may be used to generate polyclonal antibodies or monoclonal antibodies (mAb) specific for fentanyl and its analogs. Various immunization strategies for generating and isolating antibodies and mAbs known to those of skill in the art maybe used.

In some embodiments, a fentanyl hapten may be used to isolate an antibody (including, for example a mAb) specific for fentanyl or a fentanyl analog. In some embodiments, a fentanyl hapten may be used to isolate an antibody generated by immunization with a fentanyl hapten-carrier conjugate. In some embodiments, a fentanyl hapten may be used to isolate an antibody specific for a corresponding target. For example, F₁, F₂, F₃, F₄, F₅, F₆, F₉, or F₁₂ may be used to isolate a fentanyl-specific antibody; in another example, F₇, F₁₁, or F₁₃ may be used to isolate a carfentanil-specific antibody; in a further example, F₈ or F₁₀ may be used to isolate an acetylfentanil-specific antibody. In some embodiments, multiple fentanyl haptens may be used to isolate an antibody specific for a single target or for multiple targets. In some embodiments, a fentanyl hapten may be used to isolate an antibody generated by immunization with the corresponding fentanyl hapten-carrier conjugate. Additionally or alternatively, a fentanyl hapten may be used to isolate an antibody generated by immunization with a different fentanyl hapten-carrier conjugate. For example, a fentanyl hapten used to isolate the antibody may be based on the same fentanyl analog as the fentanyl hapten used in the fentanyl hapten-carrier conjugate.

In some embodiments, the method includes administering the fentanyl hapten-carrier conjugate in combination with an opioid agonist or partial agonist. Exemplary opioid agonists or partial agonists include, for example, methadone, buprenorphine, etc. In some embodiments, the fentanyl hapten-carrier conjugate preferably does not interfere with the effects of an opioid agonists or partial agonist.

In some embodiments, the method includes administering the fentanyl hapten-carrier conjugate in combination with an opioid antagonist. Exemplary opioid antagonists include, for example, naloxone, nalmefene, naltrexone, etc. In some embodiments, the fentanyl hapten-carrier conjugate preferably does not interfere with the effects of an opioid antagonist.

As described in the Examples, the fentanyl hapten-carrier conjugates described herein may provide an effective fentanyl vaccine. For example, fentanyl hapten-carrier conjugates including haptens F₁, F₂, F₃, F₅, or F₆, were effective in significantly reducing (p≤0.0001) fentanyl-induced antinociception in the hot plate test at 30-minutes post-challenge; and F₁, F₂, F₃, F₄, F₅, or F₆, were effective in reducing (p≤0.05) fentanyl-induced antinociception compared to controls. (FIG. 2)

Fentanyl hapten-carrier conjugates including haptens F₁, F₂, or F₃ were effective in reducing fentanyl-induced respiratory depression (FIG. 3D), bradycardia (FIG. 3E), and distribution of fentanyl to the brain (FIG. 3F) in rats. Selected conjugates containing F₁, F₂ and F₃ haptens were effective in reducing sufentanil-induced antinociception in rats. (FIG. 4.)

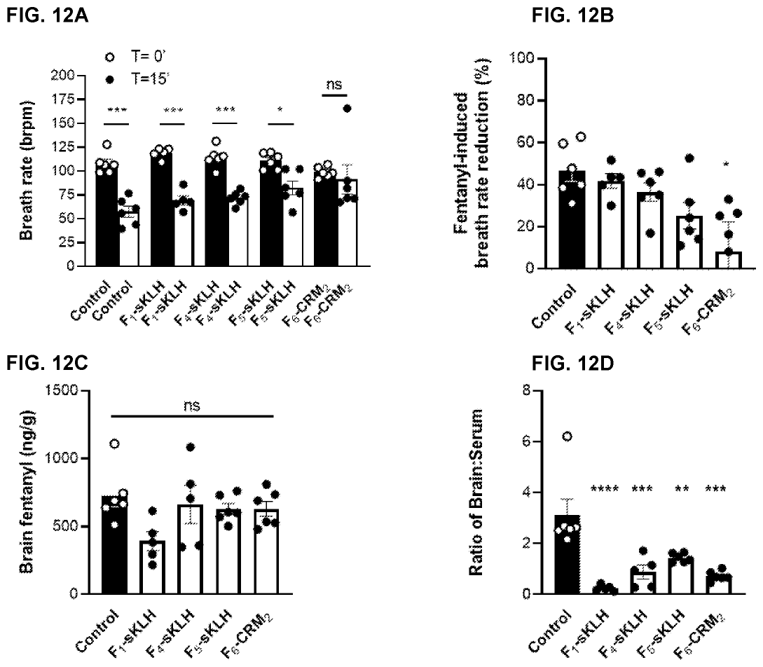

Fentanyl hapten-carrier conjugates including the F₄, F₅, or F₆ haptens conjugated to either sKLH or CRM₂ were effective in significantly reducing (p≤0.0001) effects of fentanyl antinociception in the hot plate test (FIG. 5A), respiratory depression reported as percentage (%) of oxygen saturation measured by oximetry (FIG. 5B), and bradycardia reported as heart rate measured by oximetry (FIG. 5C). (See also FIG. 12.) Fentanyl hapten-carrier conjugates including the F₆ hapten conjugated to CRM₂ was effective in reducing effects of sufentanil antinociception in the hot plate test (FIG. 5D) and multiple conjugates showed a trend towards reduced effects in sufentanil-induced respiratory depression (FIG. 5E) and bradycardia (FIG. 5F).

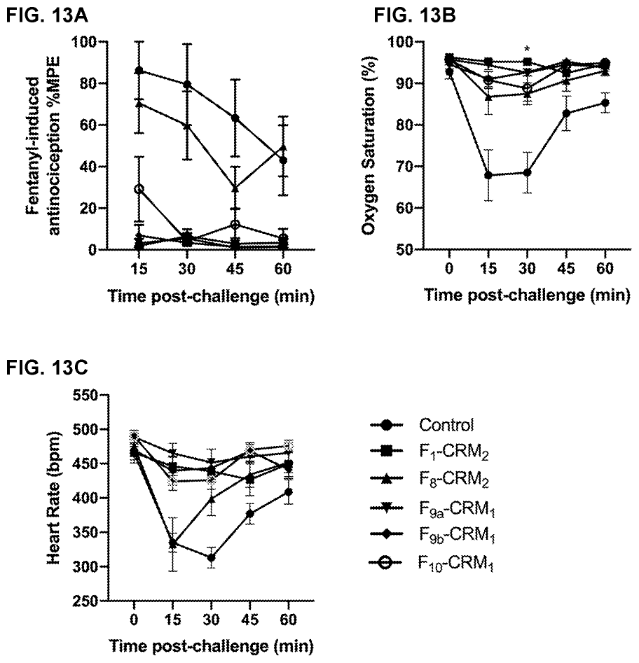

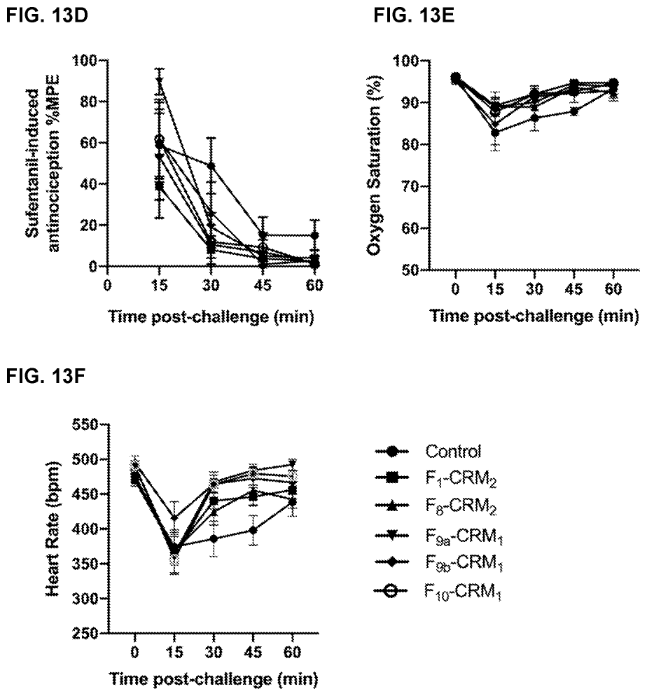

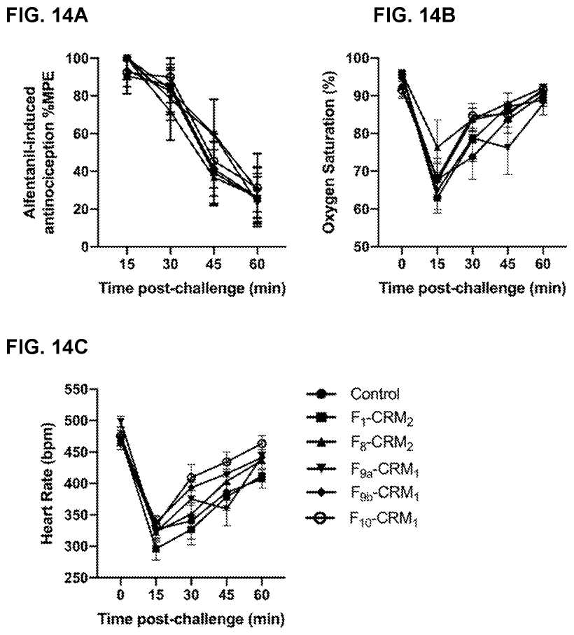

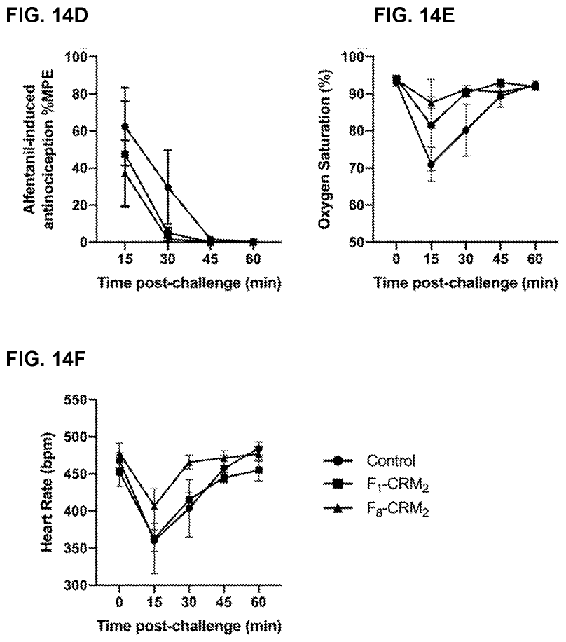

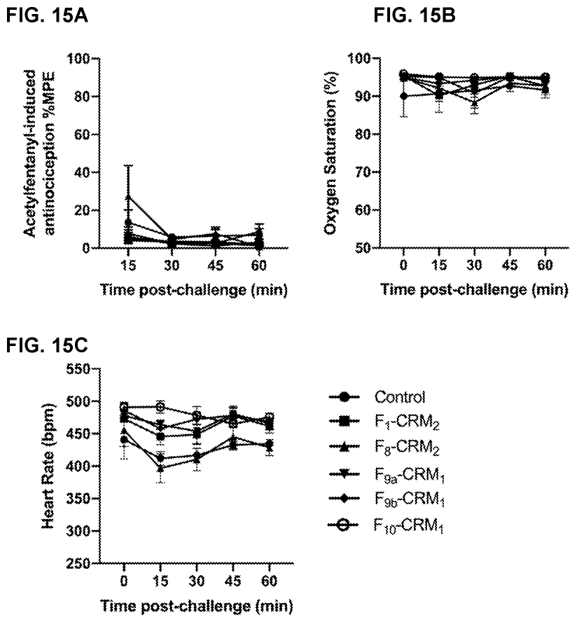

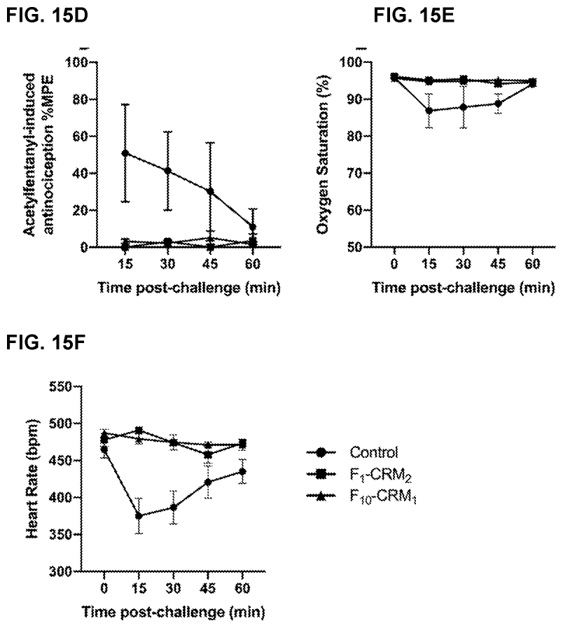

Fentanyl hapten-carrier conjugates including the F₁, F₈, F₉ₐ, F_{9b} or F₁₀ haptens conjugated to either sKLH or CRM₂ were effective in significantly reducing effects of fentanyl antinociception in the hot plate test (FIG. 13A), respiratory depression reported as percentage (%) of oxygen saturation measured by oximetry (FIG. 13B), and bradycardia reported as heart rate measured by oximetry (FIG. 13C). A fentanyl hapten-carrier conjugate including F₁ or F₁₀ also exhibited efficacy at reducing the effects of acetyl fentanyl (FIG. 15). These same fentanyl hapten-carrier conjugates were not, however, effective in reducing effects of sufentanil (FIG. 13D-FIG. 13E) or alfentanil (FIG. 14A-14C).

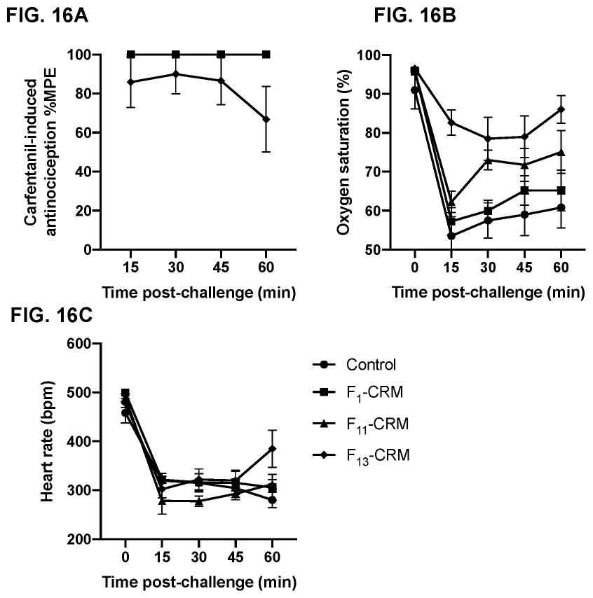

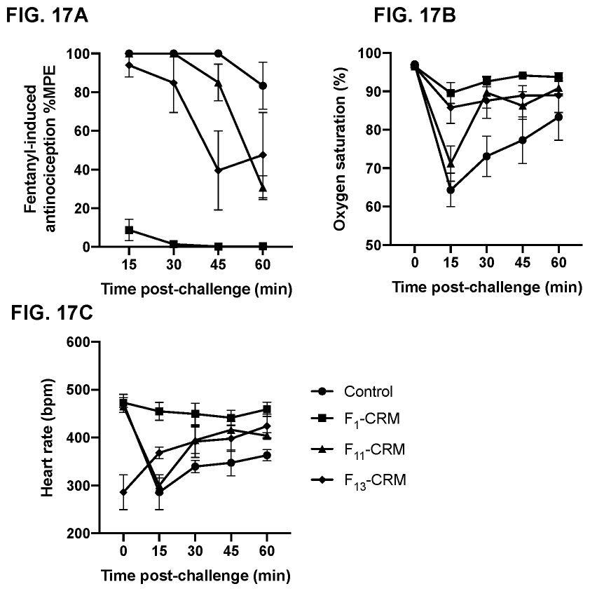

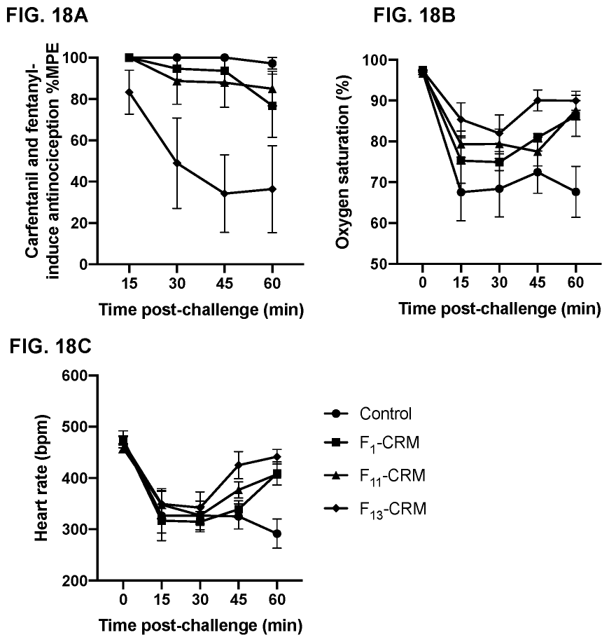

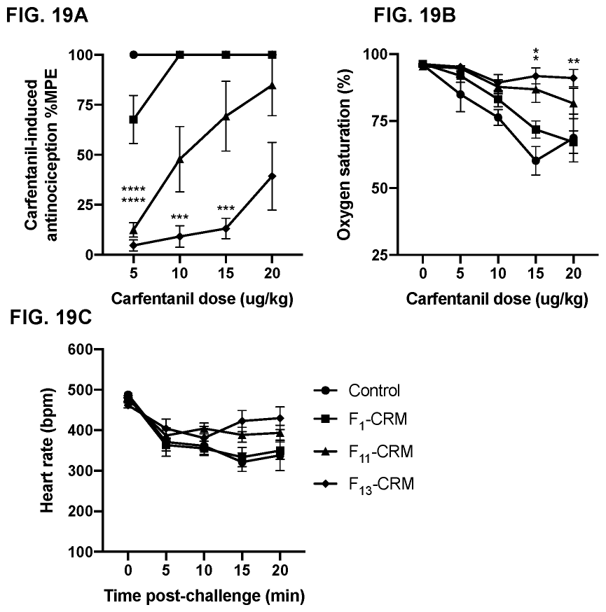

Fentanyl hapten-carrier conjugates including the F₁, F₁₁, or F₁₃ haptens conjugated to CRM were minimally or un-effective in significantly reducing effects of carfentanil antinociception in the hot plate test (FIG. 16A), respiratory depression reported as percentage (%) of oxygen saturation measured by oximetry (FIG. 16B), and bradycardia reported as heart rate measured by oximetry (FIG. 16C). While these same fentanyl hapten-carrier conjugates were effective at reducing at least some of the effects of fentanyl (FIG. 17), they were not, however, effective in reducing effects of a combination of carfentanil and fentanyl (FIG. 18). While these same fentanyl hapten-carrier conjugates were challenged with increasing doses of carfentanil (FIG. 19), the F₁₁- and F₁₃-containing conjugates were effective in reducing carfentanil-induced antinociception (FIG. 19A) and respiratory depression (FIG. 19B). Specifically F₁₃-CRM was effective against the highest doses of carfentanil.

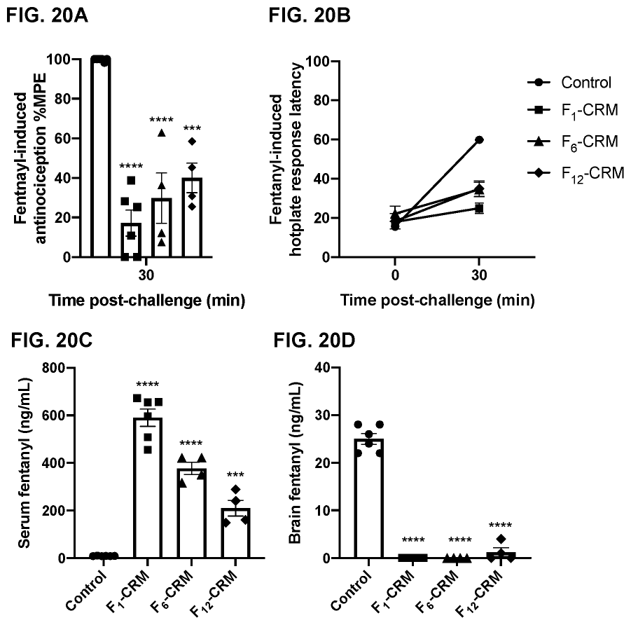

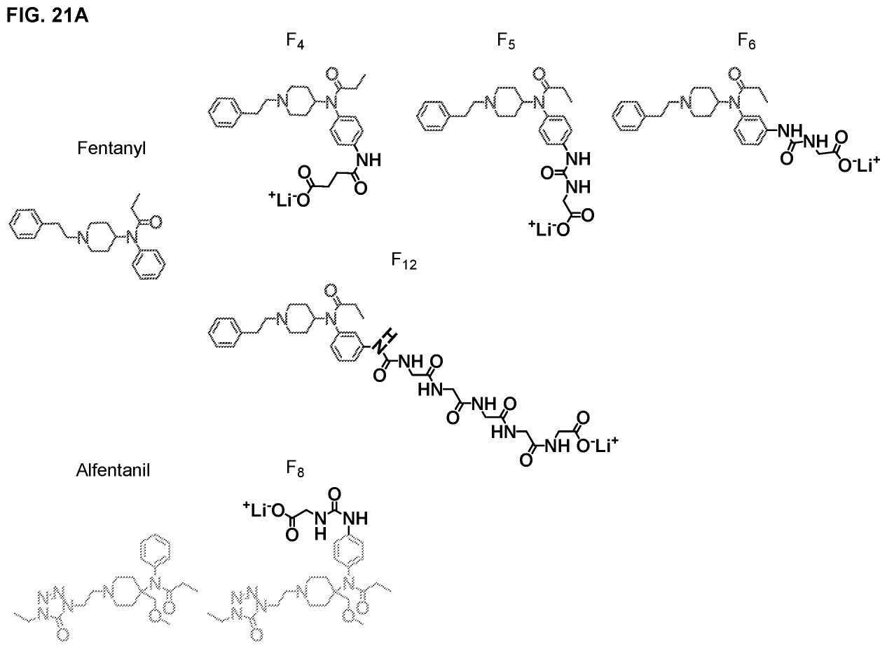

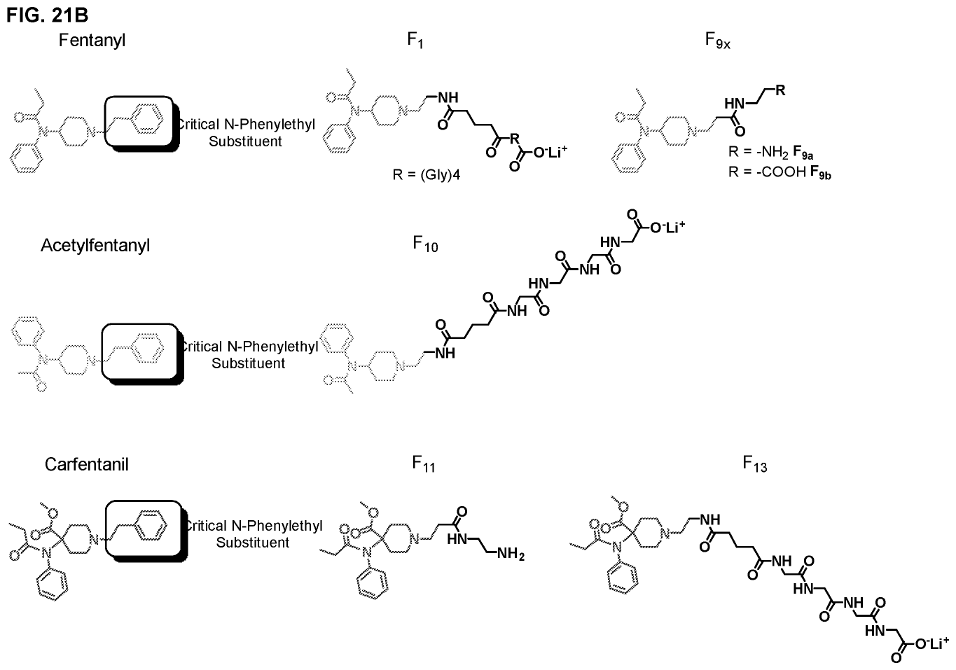

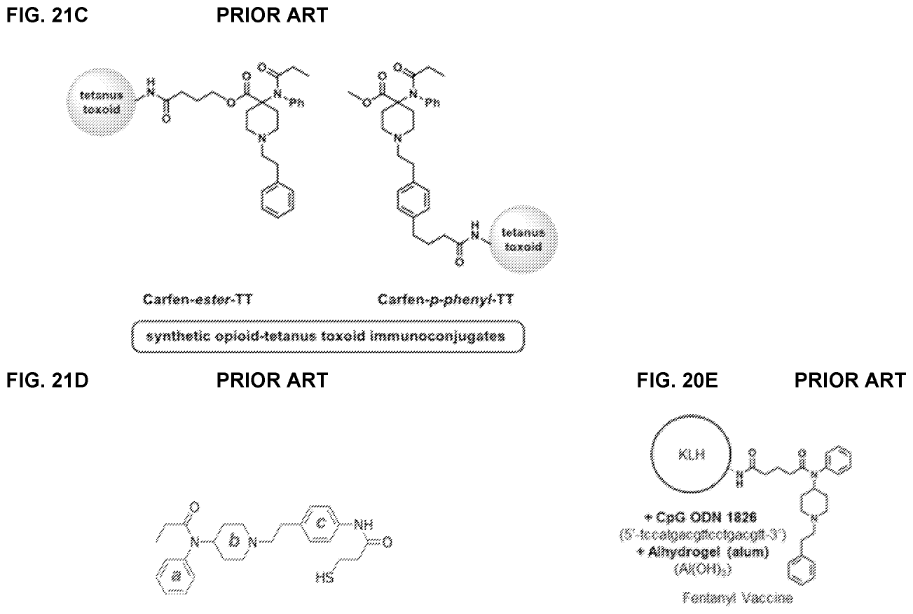

Fentanyl hapten-carrier conjugates including the F₁, F₆, or F₁₂ haptens conjugated to CRM were effective in significantly reducing effects of fentanyl antinociception in the hot plate test (FIG. 20A) and altered the distribution of fentanyl in the serum and the brain (FIG. 20C-FIG. 20D) of mice.

The data in the Examples suggest that a fentanyl vaccine could be a viable option for reducing the respiratory depressive effects and cardiac toxicity of fentanyl in humans, effects which are commonly associated with fatal overdoses. Moreover, even after treatment with a fentanyl hapten-carrier conjugate including F₁, naloxone reversed fentanyl effects, showing that naloxone's ability to reverse respiratory depression was preserved and that no cross-reactivity between anti-fentanyl antibodies generated by vaccination and naloxone existed. Lack of cross-reactivity (that is, selectivity), permits clinical use of vaccines in combination with current treatment for OUD and overdose.

The data in FIG. 6 suggest that a fentanyl vaccine could be a viable treatment option for patients with an ongoing fentanyl use disorder and that it would not be necessary to first detoxify patients (like in the case of naltrexone or other opioid antagonist) prior to the initiation of an immunization regimen. In addition, data in FIG. 6 further suggest that the immunogenicity and efficacy of a fentanyl vaccine is not affected by concurrent fentanyl use, and that vaccination does not trigger withdrawal symptoms. Furthermore, in rats with ongoing fentanyl self-administration, vaccination with F₁-CRM did not induce compensation but rather decreased fentanyl intake suggesting that fentanyl hapten-carrier conjugate vaccines are safe and will not trigger over-intake of fentanyl or other synthetic analogs (for example, sufentanil or carfentanil) to overcome the effect of the vaccine.

FIG. 7 shows immunization against fentanyl does not interfere with anesthesia and rescue suggesting that when the fentanyl hapten-carrier conjugates are used as a fentanyl vaccine, patients would still be able to be successfully anesthetized including, for example, to undergo surgical or emergency procedures.

In some embodiments, including when the fentanyl hapten-carrier conjugate is used in an anti-opioid vaccine, a composition including the fentanyl hapten-carrier conjugate preferably includes an adjuvant or other immunostimulatory molecule.

The fentanyl hapten-carrier conjugate may be administered to any subject determined to benefit. For example, in some embodiments, administration of an anti-opioid vaccine may be used to treat a subject who may be exposed to an opioid or that has been exposed to an opioid. Exemplary opioids include fentanyl and fentanyl analogs (including, for example, sufentanil or carfentanil). In some embodiments, a subject may include, an individual at risk of exposure to fentanyl, a fentanyl derivative, or a fentanyl analog. Such an individual may include, for example, a soldier, a law enforcement professional, a health profession, a first responder, etc. In some embodiments, a subject may include an individual who has been diagnosed with a substance use disorder. In some embodiments, a subject may include an individual who has been diagnosed with an opioid use disorder. In some embodiments, a subject may include a pregnant mother treated for a substance use disorder including, for example, an opioid use disorder. In some embodiments, a subject may include a newborn child of a mother treated for a substance use disorder including, for example, an opioid use disorder. In some embodiments, a subject may include a pregnant mother being treated for a substance use disorder including, for example, an opioid use disorder. Additional substance use disorders in addition to an opioid use disorder may include, for example, use of substances such as tobacco, alcohol, cannabis, stimulants, benzodiazepines, cocaine, methamphetamine, etc. Such substances are often taken by opioid users to reduce symptoms of opioid withdrawal or craving for opioids, or to enhance the effects of administered opioids.

A composition including a fentanyl hapten-carrier conjugate of the present disclosure may be formulated in pharmaceutical preparations in a variety of forms adapted to the chosen route of administration. One of skill will understand that the composition will vary depending on mode of administration and dosage unit. For example, for parenteral administration, isotonic saline may be used. Other suitable carriers include, but are not limited to alcohol, phosphate buffered saline, and other balanced salt solutions. The compounds of this invention may be administered in a variety of ways, including, but not limited to, intravenous, topical, oral, subcutaneous, intraperitoneal, and intramuscular delivery. In some aspects, the compounds of the present disclosure may be formulated for controlled or sustained release. In some aspects, a formulation for controlled or sustained release is suitable for oral implantation. In some aspects, a formulation for controlled or sustained release is suitable for subcutaneous implantation. Any suitable means of achieving controlled or sustained release may be used including, for example, embedding the fentanyl hapten-carrier conjugate in a matrix of insoluble substance, the use of a reservoir device or a matrix device, cross-liking the fentanyl hapten-carrier conjugate to an ion exchange resin, etc. In some aspects, a formulation for controlled or sustained release includes a patch. A compound may be formulated for enteral administration, for example, formulated as a capsule or tablet.

Administration may be as a single dose or in multiple doses. In some embodiments, the dose is an effective amount as determined by the standard methods, including, but not limited to, those described herein. Those skilled in the art of clinical trials will be able to optimize dosages of particular compounds through standard studies. Additionally, proper dosages of the compositions may be determined without undue experimentation using standard dose-response protocols. Administration includes, but is not limited to, any of the dosages and dosing schedules, dosing intervals, and/or dosing patterns described in the examples included herewith.

The composition including a fentanyl hapten-carrier conjugate according to the present disclosure may be administered by any suitable means including, but not limited to, for example, oral, rectal, nasal, topical (including transdermal, aerosol, buccal and/or sublingual), vaginal, parenteral (including subcutaneous, intramuscular, and/or intravenous), intradermal, intravesical, intra-joint, intra-arteriole, intraventricular, intracranial, intraperitoneal, intranasal, or by inhalation.

For use as a vaccine, the composition including a fentanyl hapten-carrier conjugate will typically be administered parenterally, usually by intramuscular or subcutaneous injection. Other modes of administration, however, are also possible.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that may be employed will be known to those of skill in the art. Some variation in dosage will necessarily occur depending on the condition of the subject being treated. The person responsible for administration will, in any event, determine the appropriate dose for the individual subject. Moreover, for human administration, preparations should meet sterility, pyrogenicity, and general safety and purity standards as required by the FDA. Such preparations may be pyrogen-free.

Many suitable formulations are known, including polymeric or protein microparticles encapsulating drug to be released, ointments, gels, or solutions which may be used topically or locally to administer drug, and even patches, which provide controlled release over a prolonged period of time. These may also take the form of implants.

The compounds may also be provided in a lyophilized form. Such compositions may include a buffer, for example, phosphate buffered saline (PBS), for reconstitution prior to administration, or the buffer may be included in the lyophilized composition for reconstitution with, for example, water. The lyophilized composition may also include compounds to stabilize the formulation, such as sugars including sucrose, lactose, maltose, and mannitol. The lyophilized composition may be provided in a syringe, optionally packaged in combination with the buffer for reconstitution, such that the reconstituted composition may be immediately administered to a patient.

In some embodiments, the fentanyl vaccine may be formulated to administration via a device. For example, the fentanyl vaccine could be administered in a device for delivery via intraossal (IO), intramuscular (IM), subcutaneous (SC), intradermal (ID), or intranasal (IN) routes of administration.

In some embodiments, for example, a composition including a fentanyl hapten-carrier conjugate according to the present disclosure may preferably be given before a subject is exposed to an opioid to prevent or mitigate the effects of opioid exposure.

Additionally or alternatively, a composition including a fentanyl hapten-carrier conjugate according to the present disclosure may be given after a subject is exposed to an opioid to reverse or mitigate the effects of a subsequent opioid exposure. Additionally or alternatively, a composition including a fentanyl hapten-carrier conjugate according to the present disclosure may be given after a subject is exposed to an opioid to prevent or reduce likelihood of fatal overdose. Additionally or alternatively, a composition including a fentanyl hapten-carrier conjugate according to the present disclosure may be given as prophylaxis measure to those at risk of mass casualty incidents or chemical attacks, or other form of deliberate poisoning.

Effective concentrations and amounts may be determined for each application herein empirically by testing the compounds in known in vitro and in vivo systems, such as those described herein, dosages for humans or other animals may then be extrapolated therefrom.

A composition as described herein may be administered at once, or may be divided into a number of smaller doses to be administered at intervals of time. For example, compositions may be administered repeatedly, for example, at least 2, 3, 4, 5, 6, 7, 8, or more times, or may be administered by continuous infusion. It is understood that the precise dosage and duration of treatment may be determined empirically using known testing protocols or by extrapolation from in vivo or in vitro test data. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions and methods.

In some therapeutic embodiments, an “effective amount” of an agent (for example, a fentanyl hapten-carrier conjugate) is an amount that results in a reduction of at least one pathological parameter upon exposure to an opioid. Exemplary parameters include respiratory depression and bradycardia. Thus, for example, in some aspects, an effective amount is an amount that is effective to achieve a reduction of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% compared to the expected reduction in the parameter in an individual not treated with the agent.

In some aspects, the administration of a fentanyl hapten-carrier conjugate may allow for the effectiveness of a lower dosage of other therapeutic modalities when compared to the administration of the other therapeutic modalities alone, providing relief from the toxicity observed with the administration of higher doses of the other modalities. For example, in some aspects, pre-administration of a fentanyl hapten-carrier conjugate vaccine may be used to decrease the amount of naloxone, nalmefene, or an anti-opioid antibody that would otherwise be needed to protect a patient.

The present invention is illustrated by the following examples. It is to be understood that the particular examples, materials, amounts, and procedures are to be interpreted broadly in accordance with the scope and spirit of the invention as set forth herein.

### Exemplary Fentanyl Hapten Aspects

A1. A fentanyl hapten comprising

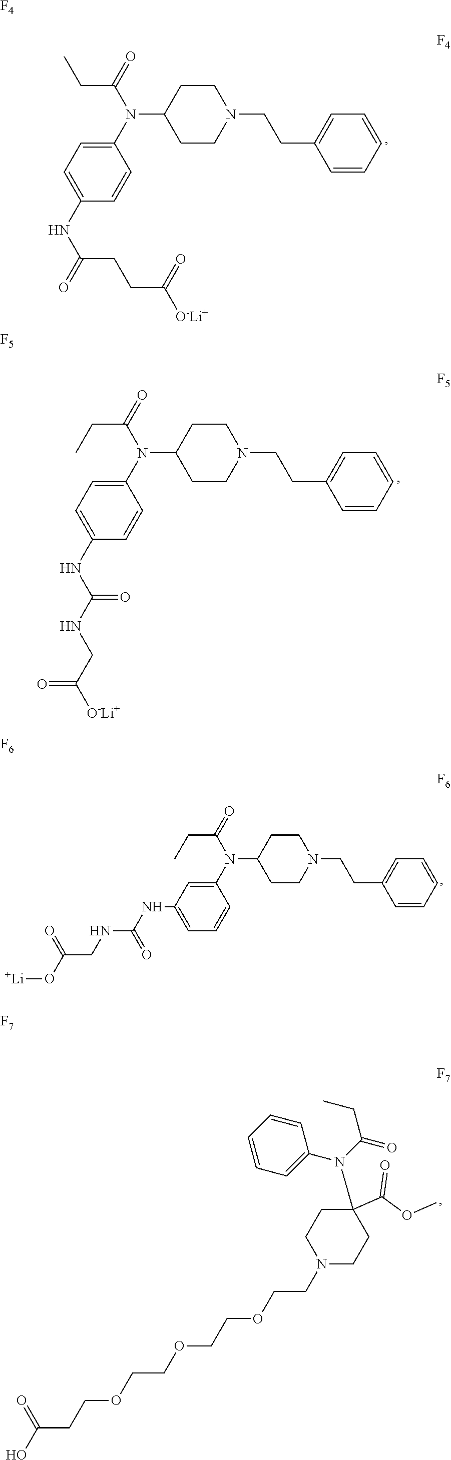

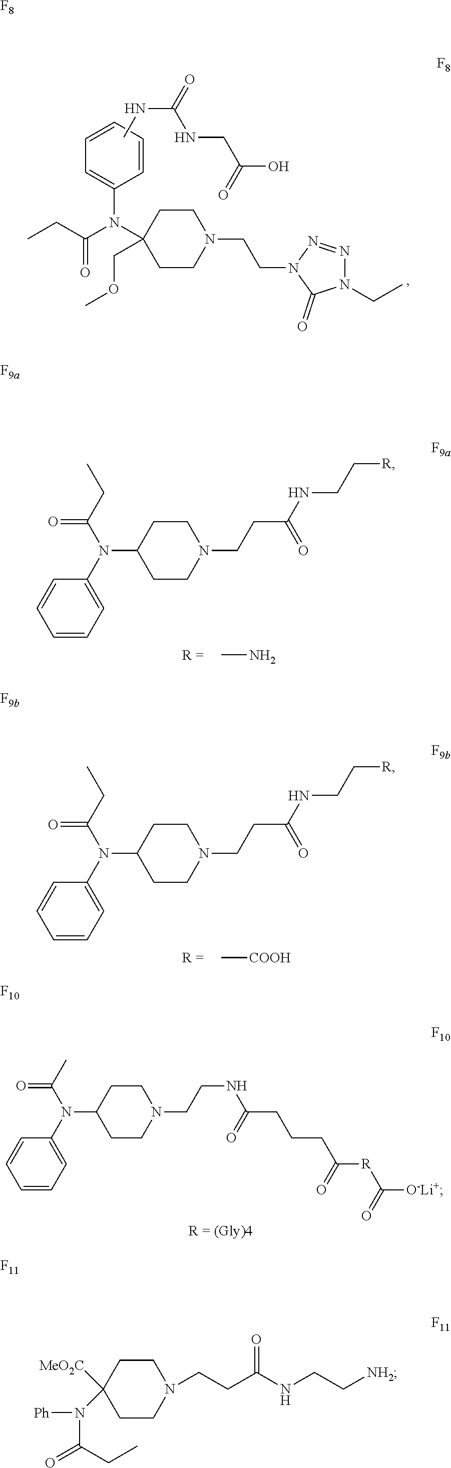

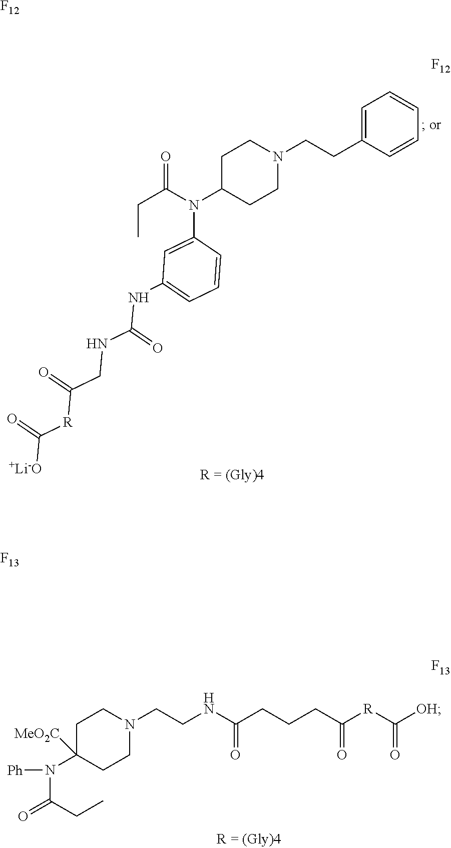

or
a combination thereof.
A2. A composition comprising the fentanyl hapten of Aspect A1.
A3. A method of making the fentanyl hapten of Aspect A1.

### Exemplary Fentanyl Hapten Conjugate Aspects

B1. A fentanyl hapten-carrier conjugate comprising

a fentanyl hapten comprising

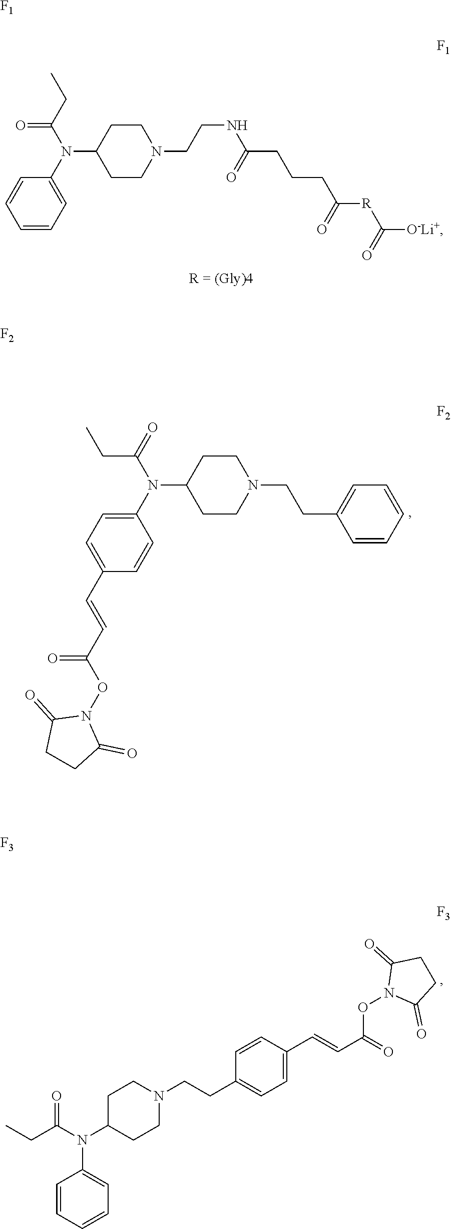

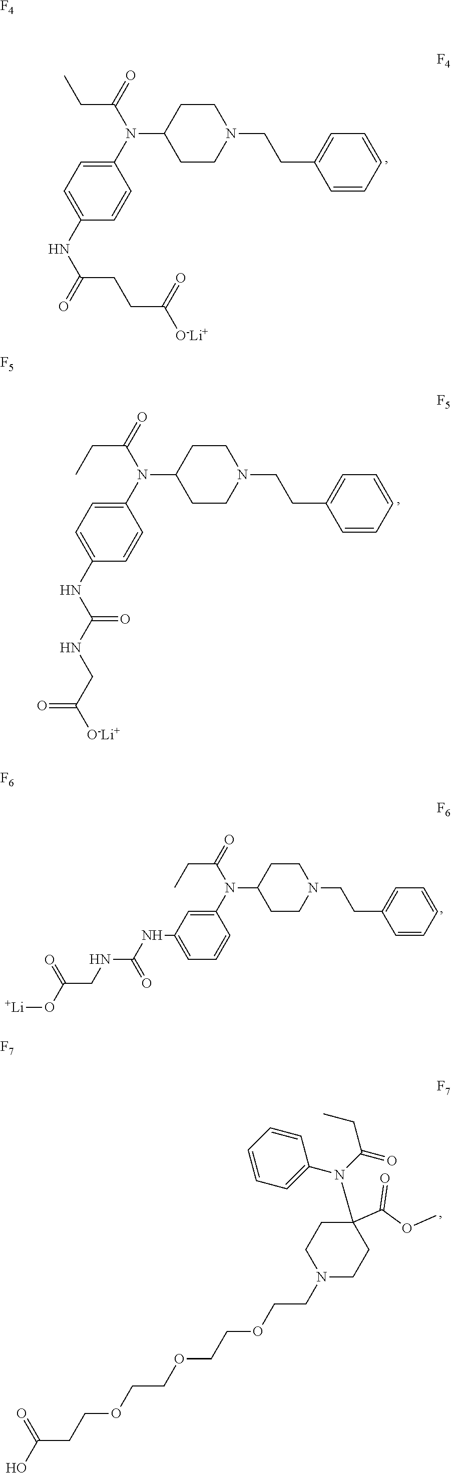

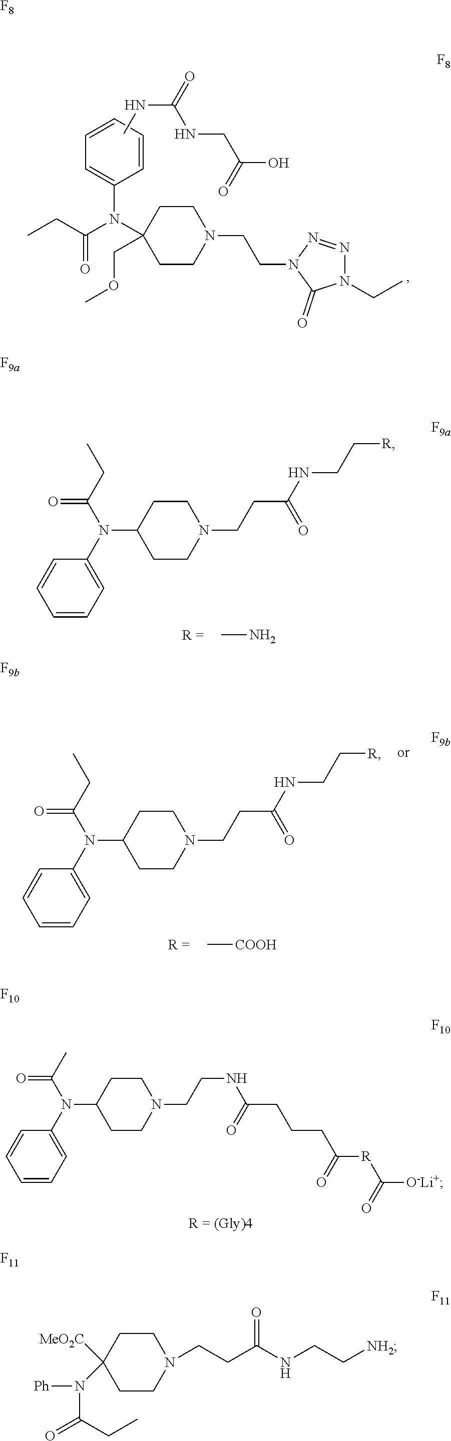

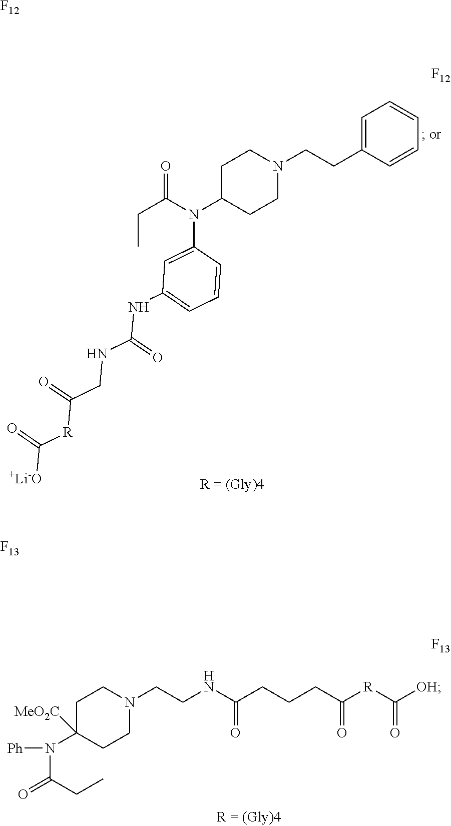

and

an immunogenic carrier, wherein the fentanyl hapten is conjugated to the immunogenic carrier.

B2. The fentanyl hapten-carrier conjugate of Aspect B1, wherein the immunogenic carrier comprises a carrier selected from bovine serum albumin (BSA), ovalbumin (OVA), keyhole limpet hemocyanin (KLH); CRM; a liposome, tetanus toxoid (TT); a peptide; macro-, micro-, and nano-particles or combinations thereof; a carbon-based particle; a nanocarrier; a protein of viral, bacterial, or synthetic origin; or another immunogenic component; or a mixture or combination thereof.
B3. The fentanyl hapten-carrier conjugate of Aspect B1 or B2, wherein the immunogenic carrier comprises KLH.
B4. The fentanyl hapten-carrier conjugate of any one of Aspects B1 to B3, wherein the immunogenic carrier comprises GMP grade subunit KLH (sKLH).
B5. The fentanyl hapten-carrier conjugate of any one of Aspects B1 to B4, wherein the immunogenic carrier comprises CRM.
B6. The fentanyl hapten-carrier conjugate of any one of Aspects B1 to B5, wherein the fentanyl hapten is conjugated to the immunogenic carrier through coupling chemistry.
B7. The fentanyl hapten-carrier conjugate of any one of Aspects B1 to B6, wherein the fentanyl hapten-carrier conjugate comprises two or more different haptens and wherein each fentanyl hapten is conjugated to a separate one of two or more immunogenic carriers.
B8. The fentanyl hapten-carrier conjugate of Aspect B7, wherein two or more different haptens are selected from F₁, F₂, F₃, F₄, F₅, F₆, F₇, F₈, F₉ₐ, F_{9b}, F₁₀, F₁₁, F₁₂, or F₁₃.
B9. The fentanyl hapten-carrier conjugate of any one of Aspects B1 to B8, wherein the fentanyl hapten-carrier conjugate comprises two or more different haptens and wherein each fentanyl hapten is conjugated to a single immunogenic carrier.
B10. The fentanyl hapten-carrier conjugate of Aspect B9, wherein the two or more different haptens are selected from F₁, F₂, F₃, F₄, F₅, F₆, F₇, F₈, F₉ₐ, F_{9b}, F₁₀, F₁₁, F₁₂, or F₁₃.
B11. The fentanyl hapten-carrier conjugate of any one of Aspects B1 to B10, wherein the number of fentanyl hapten molecules per immunogenic carrier molecule is at least 1.
B12. The fentanyl hapten-carrier conjugate of any one of Aspects B1 to B11, wherein the number of fentanyl hapten molecules per immunogenic carrier molecule is at least 5, at least 10, at least 15, at least 20, at least 30, at least 40, or at least 50.
B13. The fentanyl hapten-carrier conjugate of any one of Aspects B1 to B12, wherein the number of hapten molecules per carrier molecule is up to 30, up to 40, or up to 50, up to 100, up to 200, or up to 300

### Exemplary Composition Aspects

C1. A composition comprising the fentanyl hapten of Aspect A1 or the fentanyl hapten-carrier conjugate of any one of the Exemplary Fentanyl Hapten Conjugate Aspects (B1 to B13).
C2. The composition of Aspect C1, wherein the composition further comprises an adjuvant.
C3. The composition of Aspect C2, wherein the adjuvant comprises an aluminum adjuvant or aluminum-based adjuvant, complete Freund's adjuvant (CFA), incomplete Freund's adjuvant (IFA), a phytol-based adjuvant, a toll like receptor (TLR) agonist, a oligomerization domain (NOD)-like receptor (NLR) agonist, a RIG-I-like receptor (RLR) agonist, a C-type lectin receptor (CLR) agonist, degradable nanoparticles, or non-degradable nanoparticles, or combinations thereof.
C4. The composition of Aspect C3, wherein the fentanyl hapten or fentanyl hapten-carrier conjugate is adsorbed on an aluminum-based adjuvant.
C5. The composition of Aspect C3, wherein the composition comprises a toll like receptor (TLR) agonist and wherein the fentanyl hapten or fentanyl hapten-carrier conjugate is adsorbed on an aluminum-based adjuvant.
C6. The composition of any one of Aspects C1 to C5, wherein the composition comprises at least two fentanyl haptens or at least two fentanyl hapten-carrier conjugates.
C7. The composition of any one of Aspects C1 to C5, the composition further comprising an opioid-based hapten.
C8. The composition of any one of Aspects C1 to C7, the composition further comprising a drug-based hapten designed to target a stimulant.
C9. The composition of any one of Aspects C1 to C8, the composition comprising a buffer or a pharmaceutically acceptable carrier or both.

### Exemplary Method of Making Aspects

D1. A method of making the fentanyl hapten of Aspect A1 or the fentanyl hapten-carrier conjugate of any one of the Exemplary Fentanyl Hapten Conjugate Aspects (Aspects B1 to B13).
D2. The method of Aspect D1, the method comprising using a synthesis described in Example 1, Example 2, Example 3, or Example 4.
D3. The method of Aspect D1 or D2, wherein the method comprises conjugating the fentanyl hapten to the immunogenic carrier through coupling chemistry.
D4. A method of making the composition of any one of the Exemplary Composition Aspects (Aspects C1 to C6).

### Exemplary Method of Using Aspects

E1. A method comprising administering the fentanyl hapten of Aspect A1, the fentanyl hapten-carrier conjugate of any one of the Exemplary Fentanyl Hapten Conjugate Aspects (B1 to B13), or the composition of any one of the Exemplary Composition Aspects (C1 to C6) to a subject.
E2. The method of Aspect E1, wherein the subject is an individual at risk of exposure to fentanyl, a fentanyl derivative, or a fentanyl analog.
E3. The method of Aspect E2, wherein the individual at risk of exposure to fentanyl, a fentanyl derivative, or a fentanyl analog is a soldier, a law enforcement professional, a health profession, or a first responder.
E4. The method of Aspect E2 or E3, wherein the fentanyl analog comprises mefentanyl, α-mefentanyl, ohmefentanyl, phenaridine, carfentanil, lofentanil, sufentanil, alfentanil, brifentail, remifentanil, trefentanil, mirfenantil, alfentanil, acetylfentanyl, brorphine, a novel fentanyl analog, or a combination thereof.
E5. The method of Aspect E1, wherein the subject is an individual who has been diagnosed with a substance use disorder.
E6. The method of Aspect E1, wherein the subject is an individual who has been diagnosed with an opioid use disorder.
E7. The method of any one of Aspects E1 to E6, wherein the method comprises administering multiple doses of the fentanyl hapten-carrier conjugate or the composition comprising the fentanyl hapten-carrier conjugate to the subject.
E8. The method of any one of Aspects E1 to E7, wherein the method comprises administering the fentanyl hapten-carrier conjugate to the subject in combination with an opioid agonist or partial agonist.
E9. The method of any one of Aspects E1 to E8, wherein the method comprises administering multiple fentanyl hapten-carrier conjugates to the subject simultaneously.
E10. The method of any one of Aspects E1 to E9, the fentanyl hapten-carrier conjugate to the subject in combination with an opioid antagonist.
E11. The method of any one of Aspects E1 to E10, wherein the method comprises administering the fentanyl hapten-carrier conjugate to the subject in combination with an opioid-based hapten.
E12. The method of any one of Aspects E1 to E11, wherein the method comprises administering the fentanyl hapten-carrier conjugate to the subject in combination with a drug-based hapten designed to target a stimulant.
E13. The method of any one of Aspects E1 to E12, wherein the method comprises a multivalent immunization strategy.
E15. The method of Aspect E1, wherein the method further comprises isolating an antibody from the subject.
E16. The method of Aspect E16, wherein the antibody is specific to the fentanyl hapten, fentanyl, and/or a fentanyl analog.

### EXAMPLES

All reagents, starting materials, and solvents used in the following examples were purchased from commercial suppliers (such as Sigma Aldrich, St. Louis, Mo.) and were used without further purification unless otherwise indicated.

### Example 1—F₁ to F₇

This Example describes the development of vaccine formulations containing a series of fentanyl-based haptens conjugated to GMP-grade carrier proteins. Conjugate vaccines were characterized for their biophysical properties, and then tested in pre-clinical models of opioid behavior and toxicity. Mice and rats were immunized intramuscularly (i.m.), and then challenged with single or multiple subcutaneous (s.c.) doses of fentanyl and its analogs. Before and after drug administration, all animals were tested for antinociception in the hotplate assay as well as respiratory depression and bradycardia by means of an oximeter. Finally, the lead vaccine formulation was tested in a fentanyl intravenous self-administration (FSA) rat model. The most promising vaccine formulations were effective in blocking fentanyl-induced antinociception, respiratory depression, and bradycardia in mice and rats. Polyclonal antibodies showed high affinity for fentanyl but also cross reactivity to its analogs, such as sufentanil. Because of their selectivity, vaccines did not interfere with most commonly used anesthetics, nor with off target opioids used in treatment of opioid use disorders or pain management (for example, methadone, buprenorphine, naloxone, naltrexone). Vaccination was effective in reducing ongoing FSA in rats. Furthermore, immunized rats did not increase fentanyl intake to overcome vaccine efficacy during the FSA protocol. These pre-clinical data support translation of vaccines as a viable strategy to counteract illicit use and to prevent toxicity and fatal overdoses from fentanyl and its analogs.

### Materials and Methods

Drugs and reagents. Fentanyl, sufentanil, alfentanil, remifentanil, and other controlled substances were obtained from the University of Minnesota Pharmacy. Drug doses and concentrations are expressed as weight of the free base. Subunit Keyhole Limpet Hemocyanin (sKLH) was purchased as GMP-grade source (Biosyn, Carlsbad, Calif.), cross-reactive material (CRM) from diphtheria toxin was purchased either as E. coli-expressed CRM (EcoCRM) (Fina Biosolutions, Rockville, Md.) or CRM₁₉₇ (PFEnex, San Diego, Calif.). CRM from these different sources is described as CRM₁ and CRM₂, respectively.

Animals. All studies were approved by the University of Minnesota and the Hennepin Healthcare Research Institute Animal Care and Use Committee. Both institutions are AALAC-certified. Adult male BALB/c mice (Jackson Laboratories, Bar Harbor, Me.) and adult male Sprague-Dawley rats (Envigo, Indianapolis, Ind.) were housed in standard 12/12 hours light/dark cycle and fed ad libitum. Mice were 6 weeks old on arrival. Rats were 2 months old on arrival for drug challenge studies, and 9-10 weeks old for drug self-administration studies. Most animals were immunized immediately after 1 week of habituation. Rats in the self-administration experiment were immunized after self-administration behavior was trained and stable (see below).

Synthesis of the F₁ hapten. The F₁ hapten was synthesized as depicted in Scheme 1 (FIG. 1D), and as described below. Reductive amination of commercially available norfentanyl (1) was conducted with N-Boc-2-aminoacetaldehyde in a solution of sodium triacetoxyborohydride and 1,2-dichloroethane to afford 3 in good yield. Deprotection of 3 with trifluoroacetic acid in dichloromethane at room temperature afforded free amine 4. Acylation of 4 with methyl 5-chloro-5-oxopentanoate in the presence of triethylamine in dichloroethane gave acylated product 5 that was readily purified using normal phase chromatography. This step was important to provide clean precursor 6 following a simple hydrolysis of the methyl ester with lithium hydroxide. Previous attempts to prepare intermediate 6 using glutaric anhydride resulted in impure amino acid adducts that were extremely difficult to purify. Peptide coupling of 6 with tetraglycine methyl ester using (benzotriazol-1-yloxy)tris(dimethylamino)phosphonium hexafluorophosphate and triethylamine in dimethylformamide provided key intermediate 7 in moderate yield. Base hydrolysis of 7 with lithium hydroxide in an equal mixture of water, tetrahydrofuran, and methanol afforded target hapten F1 in quantitative yield as a white solid. The identify and purity of the final product was verified by mass spectrometry and NMR.

Synthesis of the F₂ hapten. The F₂ hapten was synthesized as depicted in Step (a) to Step (c) of FIG. 1E, and as described below.

Synthesis of (1): Fentanyl-A-4-Br (270 mg, 0.6 mmol) and tert-butyl acrylate (768 mg, 6 mmol, 10 equivalents were dissolved in Et3N (2 mL) and dimethylformamide (DMF) (3 mL). The solution was deoxygenated by bubbling with dry N₂ gas for 30 min, then a DMF solution containing Pd(OAc)₂ (13.5 mg, 0.06 mmol, 0.1 eq) and ethylenebis(diphenylphosphine) (23.9 mg, 0.06 mmol, 0.1 eq) was injected through a needle. The reaction mixture was further bubbled for 30 min, then heated to 140° C. and left stirring overnight. The reaction mixture was cooled to room temperature and ether (100 mL) was added. The organic phase was passed through a neutral alumina column to remove the Pd catalyst, and washed with 1 M NaOH, water and brine. The organic phase was dried over MgSO₄ and concentrated under vacuum to remove any remaining solvents and triethylamine. Additional ether was added, and the product was purified by precipitation in ether using HCl in ethanol solution, the product was filtered and dried and compound (1) was isolated as a white solid. (320 mg, yield 98%).

Synthesis of (2): compound (1) (320 mg, 0.69 mmol) was dissolved in 6.3 mL CH₂Cl₂, followed by slow addition of trifluoroacetic acid (0.70 mL). The solution was stirred at room temperature for 24 hours. 1 N HCl (28 mL) was added, and the reaction mixture was extracted with dichloromethane (14 mL) three times. The organics were combined, washed with water and brine, and were evaporated under high vacuum to afford pure (2) as a yellow oil (267 mg, 94%).

Synthesis of (3): compound (2) (20 mg, 0.05 mmol) was dissolved in 10 ml of dichloromethane (DCM). Ethyl-3-(3-dimethylaminopropyl)carbodiimide (EDC)-HCl (12.5 mg, 0.064 mmol) and N-Hydroxysuccinimide (NHS) (8.5 mg, 0.074 mmol) were added to the solution and the reaction was stirred for 18 hours. Work Up: Ethyl acetate (15 mL) and water (25 mL) were added to the reaction mixture and the mixture was stirred for 10 min. The mixture was transferred to a separatory funnel; the organic layer was extracted with ethyl acetate, washed with brine, and dried with anhydrous magnesium sulfate. The solvent was removed under reduced pressure to give (3), that was used without further purification.

Synthesis of the F₃ hapten. The F₃ hapten was synthesized as depicted in Step (d) to Step (f) of FIG. 1F, and as described below.

Synthesis of (4): (Step d) Fentanyl-C-4-Br (540 mg, 1.2 mmol) and tert-butyl acrylate (1.536 mg, 12 mmol, 10 eq) was dissolved in Et₃N (5 mL) and DMF (8 mL). The solution was deoxygenated by bubbling with dry N₂ gas for 30 min, then a DMF solution containing Pd(OAc)₂ (27 mg, 0.12 mmol, 0.1 eq) and ethylenebis(diphenylphosphine) (48 mg, 0.12 mmol, 0.1 eq) were added. The reaction mixture was further bubbled for 30 min, then heated to 130° C., and left stirring overnight. The reaction mixture was cooled to room temperature and ether (200 mL) was added. The organics were washed with saturated NaHCO₃ solution, water and brine. The organics were dried over MgSO₄ and were concentrated under vacuum to remove any remaining solvents and triethylamine. The crude product was purified by silica column DCM 2% MeOH to a 90% yield of pure (4).

Synthesis of (5): (Step e) compound (4) (270 mg, 0.6 mmol) was dissolved in dichloromethane (20 mL) with slow addition of trifluoroacetic acid (1 mL). The solution was stirred at room temperature for 24 hours. 1 N HCl (40 mL) was added, and the reaction mixture was extracted with dichloromethane (20 mL) three times. The organics were combined, washed with water and brine, and was evaporated under high vacuum to afford pure (5) (95%).

Synthesis of (6): (Step f) compound (5) (100 mg, 0.245 mmol) was dissolved in 10 ml of DCM. EDC-HCl (61 mg, 0.319 mmol) and NHS (42 mg, 0.368 mmol) were added to the solution and the reaction was stirred for 18 hours. Ethyl acetate (15 mL) and water (25 mL) were added to the reaction mixture and the mixture was stirred for 10 min. The mixture was transferred to a separatory funnel; the organic layer was extracted with ethyl acetate, washed with brine, and dried with anhydrous magnesium sulfate. The solvent was removed under reduced pressure to give (6), that was used without further purification.

Synthesis of the F₇ hapten. The F₃ hapten was synthesized as depicted in Step (g) to Step (k) of FIG. 1H, and as described below.

Synthesis of (7): (Step g) 1-Benzyl-4-(phenylamino)piperidine-4-carbonitrile (2.00 g, 6.86 mmol) was dissolved in 1,2 dichloroethane (26 mL) at room temperature under a nitrogen atmosphere. Propionyl chloride (3.18 g, 34.3 mmol) in 4 mL of 1,2 dichloroethane was added dropwise to the solution. The addition funnel was rinsed with 1 mL of 1,2 dichloroethane. The reaction mixture was heated at reflux for 24 hours. The mixture was allowed to cool to room temperature, and volatiles were evaporated to provide a crude compound 7.

Synthesis of (8): (Step h) The resulting solid 7 was dissolved in 30 mL of 3 M methanolic hydrogen chloride. The solution was stirred at room temperature for 2 days. Water (32 mL) was added, and the solution was stirred at room temperature for 4 hours. The methanol was evaporated, and the aqueous solution was adjusted to pH 7.5 by adding sodium carbonate. The aqueous mixture was extracted three times with chloroform. The organic layer was washed with brine and then dried over magnesium sulfate. The solution was filtered and the solvent was removed under reduced pressure to yield 3.3 grams of a brown oil. The oil was dissolved in hot isopropanol (20 mL), and a solution of oxalic acid dihydrate (1.028 g, 8.00 mmol) in 20 mL of isopropanol was added. The mixture was cooled at 4° C. The resultant precipitate was washed with cold isopropanol followed by diethyl ether. The solids were air-dried to give 2.8 grams of compound 8 as a white powder in an 85% yield.

Synthesis of (9): (Step i) 8 (1.00 g, 2.12 mmol) and ammonium formate (0.67 g, 10.6 mmol) were dissolved in 25 mL of methanol. The solution was sparged with nitrogen for 1 hour. Palladium (10%) on carbon (0.2 g) was added to the solution, and the mixture was heated at reflux for 3 hours. The mixture was allowed to come to room temperature and was stirred overnight. The mixture was filtered through Celite and concentrated under reduced pressure followed with subsequent addition of dichloromethane and then extracted with sodium bicarbonate (sat.) solution. The organic layer was separated, and the aqueous solution was washed two times with dichloromethane. The organic layers were combined and washed with brine, dried over magnesium sulfate, filtered and the solvent was removed under reduced pressure to provide 0.8 grams of 9 as a brown oil. This crude material was used without further purification.

Synthesis of (10): (Step j) 9 (250 mg, 0.86 mmol) and 4-bromo-PEG₃-CH₂CH₂COOtBu (550 mg, 1.3 mmol) was dissolved in 10 mL DMF, followed by addition of N,N-diisopropylethylamine (230 μL g, 1.3 mmol). The reaction was kept stirring at 60° C. for 24 hours. The reaction mixture was cooled to room temperature before ethyl ether (5 mL) was added, and the organics were washed with 1 N NaOH (2 mL×3), water (2 mL×3) and brine (2 mL×3). The organics were then dried over MgSO₄ and concentrated under vacuum to yield 200 mg of 10.

Synthesis of (11): (Step k) 100 mg of 10 was dissolved in CH₃CN (1 mL) at room temperature. To this solution aqueous phosphoric acid (85 wt %, 1 mL) was added. The mixture was stirred for 6 hours, and LC-MS assay showed the reaction was complete. Water (3 mL) was added and the solution was neutralized with NaOH solution. The mixture was extracted with ethyl acetate (3×3 mL). The combined ethyl acetate phase was dried over magnesium sulfate and concentrated in vacuo to give the desired product 11 (hapten F₇).

Synthesis of the F₄, F₅, and F₆ haptens. The F₄ (structure 8), F₅ (structure 6b), and F₆ (structure 6a) haptens were synthesized as depicted in FIG. 1G, and as described below.

2a: 2: tert-Butyl (3-((1-phenethylpiperidin-4-yl)amino)phenyl)carbamate: To a solution of N-Boc-m-phenylenediamine 1a (400 mg, 1.92 mmol), 1-phenethyl-4-piperidone (390 mg, 1.92 mmol) and poly(methylhydrosiloxane) (7.30 g, 3.84 mmol) in MeOH (20 mL), SnCl₂ (72 mg, 0.38 mmol) was added at room temperature and the reaction which resulted was heated to 70° C. and stirred for 16 hours. The residue was subjected to chromatography on silica gel using 0-100% MeOH in DCM to furnish amine 2a (357 mg, 47%). ¹H NMR (300 MHz, CDCl₃) δ 7.35-7.15 (m, 4H), 7.05 (t, J=8.0 Hz, 1H), 6.86 (bs, 1H), 6.48 (dd, J=7.9, 1.4 Hz, 1H), 6.38 (bs, 1H), 6.28 (dd, J=8.0, 1.7 Hz, 1H), 3.57-3.47 (m, 2H), 3.38-3.27 (m, 1H), 3.00-2.90 (m, 2H), 2.87-2.76 (m, 2H), 2.66-2.56 (m, 2H), 2.30-2.15 (m, 2H), 2.13-2.03 (m, 2H), 1.51 (s, 9H). MS (ESI) m/z 396.00 [M+H]⁺.

2b: tert-Butyl (4-((1-phenethylpiperidin-4-yl)amino)phenyl)carbamate: The procedure for the synthesis of 2a was followed starting with N-boc-p-phenylenediamine 1a to provide amine 2b (504 mg, 66%). ¹H NMR (300 MHz, CDCl₃) δ 7.32-7.28 (m, 1H), 7.23-7.11 (m, 5H), 6.56 (d, J=8.8 Hz, 2H), 6.22 (bs, 1H), 3.56-3.48 (m, 2H), 3.32-3.22 (m, 1H), 2.96 (d, J=11.8 Hz, 2H), 2.87-2.77 (m, 2H), 2.65-2.57 (m, 2H), 2.20 (t, J=11.3 Hz, 2H), 2.13-2.02 (m, 2H), 1.50 (s, 9H). MS (ESI) m/z 396.00 [M+H]⁺.

3a: tert-Butyl (3-(N-(1-phenethylpiperidin-4-yl)propionamido)phenyl)carbamate: To a solution of amine 2a (249 mg, 0.48 mmol) in dry CH₂Cl₂ (10 ml), propionic anhydride (excess) was added and the reaction which resulted was stirred for 24 h at room temperature. At that time, the reaction was quenched with saturated aq NaHCO₃ (5 mL). The layers were separated and the aqueous layer was extracted with additional CH₂Cl₂ (2×20 mL). The combined organic layers were washed with brine (3×20 mL) and dried (Na₂SO₄) and concentrated under reduced pressure. The crude amide 3a was used for next step. ¹H NMR (300 MHz, CDCl₃) δ 7.31-7.13 (m, 7H), 6.76-6.71 (m, 1H), 6.69 (bs, 1H), 4.76-4.57 (m, 1H), 3.12-2.96 (m, 2H), 2.81-2.69 (m, 2H), 2.64-2.53 (m, 2H), 2.41-2.14 (m, 3H), 2.04-1.94 (m, 2H), 1.90-1.71 (m, 2H), 1.52 (s, 9H), 1.18-1.10 (m, 1H), 1.02 (t, J=7.4 Hz, 3H). MS (ESI) m/z 452.20 [M+H]⁺.

3b: tert-Butyl (4-(N-(1-phenethylpiperidin-4-yl)propionamido)phenyl)carbamate: The procedure for the synthesis of 3a was followed starting with amine 2b to provide amide 3b. ¹H NMR (300 MHz, CDCl₃) δ 7.39 (d, J=8.6 Hz, 2H), 7.29-7.13 (m, 4H), 6.99 (d, J=8.7 Hz, 2H), 6.61 (bs, 1H), 4.79-4.58 (m, 1H), 3.55-3.47 (m, 2H), 3.18-3.07 (m, 2H), 2.84-2.73 (m, 2H), 2.67-2.57 (m, 2H), 2.30-2.19 (m, 2H), 1.94 (q, J=7.4 Hz, 2H), 1.84-1.73 (m, 2H), 1.53 (s, 9H), 1.00 (t, J=7.4 Hz, 3H). MS (ESI) m/z 452.20 [M+H]⁺.

4a: N-(3-Aminophenyl)-N-(1-phenethylpiperidin-4-yl)propionamide: To a solution of 3a (crude, 0.48 mmol) in CH₂Cl₂ (10 ml; containing 0.1 mL H₂O), trifluoroacetic acid (5 ml) was added at room temperature. The reaction was allowed to stir at room temperature for 18 hours. The solvent was removed under reduced pressure and the residue was re-dissolved in ethyl acetate (EtOAc) and washed with 1N NaOH (2×10 mL). The residue was subjected to chromatography on silica gel using 0-100% CMA 80 in DCM to furnish deprotected amide 4a. ¹H NMR (300 MHz, CDCl₃) δ 7.31-7.12 (m, 6H), 6.65 (dd, J=8.0, 1.6 Hz, 1H), 6.45 (d, J=7.7 Hz, 1H), 6.40-6.35 (m, 1H), 4.70-4.55 (m, 1H), 3.72 (s, 2H), 3.00 (d, J=11.1 Hz, 2H), 2.79-2.68 (m, 2H), 2.61-2.48 (m, 2H), 2.19-2.08 (m, 2H), 2.05-1.97 (m, 2H), 1.85-1.68 (m, 2H), 1.60-1.38 (m, 2H), 1.02 (t, J=7.5 Hz, 3H). MS (ESI) m/z 352.00 [M+H]⁺.

4b: N-(4-Aminophenyl)-N-(1-phenethylpiperidin-4-yl)propionamide: The procedure for the synthesis of 4a was followed starting with amide 3b (crude, 0.63 mmol) to provide deprotected amide 4b (84 mg, 38%). ¹H NMR (300 MHz, CDCl₃) δ ¹H NMR (300 MHz, CDCl₃) δ 7.32-7.09 (m, 5H), 6.88-6.79 (m, 2H), 6.71-6.58 (m, 2H), 4.72-4.56 (m, 1H), 3.75 (s, 2H), 2.99 (d, J=11.6 Hz, 2H), 2.78-2.69 (m, 2H), 2.59-2.47 (m, 2H), 2.21-2.08 (m, 2H), 1.97 (q, J=7.5 Hz, 2H), 1.76 (d, J=11.8 Hz, 2H), 1.53-1.37 (m, 2H), 1.01 (t, J=7.5 Hz, 3H). MS (ESI) m/z 352.00 [M+H]⁺.

5a. Methyl 2-(3-(3-(N-(1-phenethylpiperidin-4-yl)propionamido)phenyl)ureido)acetate: To a solution of triphosgene (20 mg, 0.67 mmol) in CH₃CN (10 mL) at 0° C. was added triethylamine (excess). The aniline 4a (10 mg, 0.03 mmol, free base) in CH₃CN (5 ml) was added via a syringe over 10 minutes. The reaction mixture was warmed to room temperature and methyl glycinate hydrochloride (25 mg, 0.28 mmol) in THF (10 mL) was added and the reaction was allowed to stir at room temperature for 16 hours. The reaction was quenched with MeOH (5 mL) and concentrated under reduced pressure. The residue was subjected to chromatography on silica gel using 0-100% CMA-80 in CH₂Cl₂ to furnish 5a (9.7 mg, 73%). ¹H NMR (300 MHz, DMSO) δ 8.30 (s, 1H), 7.55-7.29 (m, 2H), 7.25-6.99 (m, 8H), 4.47-4.30 (m, 1H), 4.06-3.94 (m, 2H), 3.40-3.17 (m, 3H), 3.09 (s, 1H), 2.96-2.78 (m, 3H), 2.67-2.49 (m, 2H), 2.11-1.87 (m, 3H), 1.85-1.71 (m, 2H), 1.69-1.50 (m, 2H), 1.08-1.00 (m, 1H), 0.82 (t, J=7.3 Hz, 3H). MS (ESI) m/z 467.00 [M+H]⁺.

5b. Methyl 2-(3-(4-(N-(1-phenethylpiperidin-4-yl)propionamido)phenyl)ureido) acetate: The procedure for the synthesis of 5a was followed starting with aniline 4b (35 mg, 0.10 mmol) to provide compound 5b (37 mg, 79%). ¹H NMR (300 MHz, DMSO) δ 9.30 (s, 1H), 7.48 (d, J=8.7 Hz, 2H), 7.36-7.16 (m, 5H), 7.05 (d, J=8.6 Hz, 2H), 6.69 (t, J=5.9 Hz, 1H), 4.61-4.43 (m, 1H), 3.89 (d, J=5.8 Hz, 2H), 3.65 (s, 3H), 3.21-3.11 (m, 2H), 2.75 (s, 4H), 2.56-2.36 (m, 2H), 1.91-1.70 (m, 4H), 1.44-1.29 (m, 2H), 0.87 (t, J=7.4 Hz, 3H). MS (ESI) m/z 467.00 [M+H]⁺.

6a (hapten F₆): Lithium 2-(3-(3-(N-(1-phenethylpiperidin-4-yl)propionamido)phenyl)ureido)acetate: To a solution of the ester 5a (9.7 mg, 0.02 mmol) in THF/MeOH/H₂O (1 mL: 1 mL: 0.1 mL), LiOH (1 mg, 0.04 mmol) was added and the reaction was allowed to stir at room temperature for 16 hours. The reaction was evaporated to dryness under a stream of N₂ to provide the lithium salt 6a (11 mg, >100% crude). ¹H NMR (300 MHz, DMSO) δ 7.42 (s, 1H), 7.33-7.07 (m, 7H), 6.70-6.55 (m, 1H), 4.49-4.29 (m, 1H), 3.01-2.86 (m, 4H), 2.76-2.70 (m, 1H), 2.68-2.54 (m, 2H), 2.32-2.24 (m, 2H), 2.07-1.79 (m, 4H), 1.70-1.56 (m, 3H), 0.88 (t, J=7.4 Hz, 3H). MS (ESI) m/z 453.00 [M+H]⁺.

6b (hapten F₅): Lithium 2-(3-(4-(N-(1-phenethylpiperidin-4-yl)propionamido)phenyl)ureido) acetate: The procedure for the synthesis of 6a was followed starting with ester 5b (37 mg, 0.08 mmol) to provide lithium salt 6b (38.2 mg). ¹H NMR (300 MHz, DMSO) δ 7.62-7.44 (m, 2H), 7.29-7.07 (m, 5H), 6.97 (d, J=8.6 Hz, 2H), 4.45-4.31 (m, 1H), 2.91 (d, J=11.0 Hz, 2H), 2.68-2.59 (m, 2H), 2.47-2.39 (m, 3H), 1.98 (t, J=11.1 Hz, 2H), 1.84 (q, J=7.4 Hz, 2H), 1.69-1.56 (m, 3H), 1.31-1.14 (m, 2H), 0.87 (t, J=7.4 Hz, 3H). MS (ESI) m/z 453.00 [M+H]⁺.

7: Methyl 4-oxo-4-((4-(N-(1-phenethylpiperidin-4-yl)propionamido)phenyl)amino)butanoate: To an ice cold solution of compound 4b (35 mg, 0.10 mmol) in dry CH₂Cl₂ (10 mL), triethylamine (70.0 μL, 0.50 mmol) and succinic acid monomethylester chloride (15.4 μL, 0.125 mmol) was added sequentially. The reaction mixture resulted, was warmed to room temperature and stirred for 1 hour. The reaction was quenched with saturated aq. NaHCO₃ (5 mL). The layers were separated and the aqueous layer was extracted with additional CH₂Cl₂ (2×20 mL). The combined organic layers were washed with brine (3×20 mL) and dried (Na₂SO₄) and concentrated under reduced pressure. The residue was subjected to chromatography on silica gel using 0-100% CMA-80 in DCM to furnish 7 (32.0 mg, 69%) as a white solid. ¹H NMR (300 MHz, CDCl₃) δ 9.39 (s, 1H), 7.61 (d, J=8.6 Hz, 2H), 7.30-7.22 (m, 2H), 7.21-7.12 (m, 2H), 7.05 (d, J=8.6 Hz, 2H), 6.73 (dd, J=54.0, 8.6 Hz, 1H), 4.78-4.54 (m, 1H), 3.83 (s, 3H), 3.52 (s, 2H), 3.02 (d, J=11.3 Hz, 2H), 2.80-2.69 (m, 2H), 2.62-2.47 (m, 2H), 2.18 (t, J=11.4 Hz, 2H), 2.00-1.87 (m, 2H), 1.80 (d, J=10.6 Hz, 2H), 1.54-1.36 (m, 2H), 1.25 (s, 1H), 1.01 (t, J=7.4 Hz, 3H), 0.91-0.82 (m, 1H).

8 (hapten F₄): Lithium 4-oxo-4-((4-(N-(1-phenethylpiperidin-4-yl)propionamido)phenyl)amino)-butanoate The procedure for the synthesis of 6a was followed starting with ester 7 (14.8 mg, 0.03 mmol) to provide lithium salt 8 (11.6 mg). ¹H NMR (300 MHz, DMSO) δ 12.94 (s, 1H), 7.60 (d, J=8.6 Hz, 2H), 7.29-7.19 (m, 2H), 7.19-7.12 (m, 3H), 7.08 (d, J=8.6 Hz, 2H), 4.52-4.22 (m, 1H), 2.95-2.88 (m, 2H), 2.76-2.70 (m, 1H), 2.69-2.60 (m, 3H), 2.59-2.54 (m, 1H), 2.46-2.39 (m, 2H), 2.31-2.25 (m, 1H), 1.99 (t, J=11.4 Hz, 3H), 1.83 (q, J=7.4 Hz, 3H), 1.65 (d, J=12.8 Hz, 2H), 0.90-0.82 (m, 3H). MS (ESI) m/z 452.00 [M+H]⁺.

Activity of the fentanyl-based haptens at the Mu Opioid Receptor (MOR). To test whether the F₁ hapten had any functional activity at the MOR, the F₁ hapten was tested in vitro in a calcium mobilization assay involving Chinese Hamster Ovary (CHO) cells co-expressing the human MOR and Gα16, a promiscuous G protein, as previously described (Raleigh et al. PLoS One 12, e0184876 (2017)).

Conjugation of the F₁, F₄, F₅ and F₆ haptens via carbodiimide (EDAC) chemistry. The conjugations were performed according to the protocols previously described for either OXY(Gly)₄OH or M(Gly)₄OH haptens with minor modifications (Raleigh et al. J Pharmacol Exp Ther 368, 282-291 (2019), Baruffaldi et al. Mol Pharm 15, 4947-4962 (2018), Baruffaldi et al. Mol Pharm 16, 2364-2375 (2019)). Conjugations were described in detail in (Robinson et al. J Med Chem 63, 14647-14667 (2020)). Briefly, the F₁, F₄, F₅ and F₆ haptens were dissolved at a concentration of 5.2 mM in 0.1M MES buffer pH 4.5 containing 10% DMSO and were activated by carbodiimide coupling chemistry using N-ethyl-N′-(3 dimethylaminopropyl) carbodiimide hydrochloride (EDAC, Sigma-Aldrich, St. Louis, Mo.) cross-linker at a final concentration of 208 mM. The mixture was left reacting for 10 minutes at room temperature (RT). BSA, sKLH, EcoCRM or CRM₁₉₇ were added at a final concentration of 2.8 mg/ml and the reactions were stirred for the following 3 hours at room temperature (RT). The final conjugates were ultrafiltered using Amicon filters with 50 kDa or 100 kDa molecular cutoff depending on the carrier protein dimensions: after having replaced MES buffer with phosphate-buffered salite (PBS) 0.1 M pH 7.2, the resulting solutions were stored at +4° C. EcoCRM and CRM197 conjugates required the presence of 250 mM sucrose in both reacting and storage buffer as a stabilizing agent.

Conjugation of the NHS-esters of F₂ and F₃ haptens to carrier proteins. 1 mL of a solution containing either F₂ (compound 3, FIG. 1E) or F₃ (compound 6, FIG. 1F) dissolved in DMSO (5 mg/ml) was added at a rate of 20 μL per min to 5 mL of EcoCRM or sKLH (1 mg/ml) under magnetic stirring. After 1.5 hours, samples were diluted with 5 mL of buffer and purified using a dialysis membrane (50 k MWCO) in 1×PBS for 20 hours, 2 exchanges at 4° C. Sample concentration was measured using a Direct Detect spectrophotometer.

Conjugation of the F₇ hapten to carrier proteins. The F₇ hapten (compound 11, FIG. 1G) was conjugated to either sKLH or BSA. In one reaction, 11 (32 mg, 4 mg/mL) was dissolved in 8 mL of 10 mM PBS buffer. 16 mg of EDC and 11 mg of NHS was added. After 15 min sKLH (20 mg (1 mg/mL in 10 mM PBS buffer) was added slowly. The reaction was stirred for 18 hours at room temperature. The conjugate was purified by dialysis (50 kDa) using 10 mM PBS buffer. In a second reaction, the F₇ hapten (11, 30 mg, 4 mg/mL) was dissolved in 8 mL of 10 mM PBS buffer and added slowly to the solution of BSA (20 mg (1 mg/mL in 10 mM PBS buffer) using a syringe pump. The reaction was stirred for 18 hours at room temperature. The conjugate was purified by dialysis in deionized water (25 kDa MWCO) at 4° C. for 24 hours and lyophilized.

Characterization of the F₁, F₄, F₅ and F₆ conjugates via MALDI-TOF. The molecular weight (MW) of BSA and CRM-containing conjugates was assessed prior and after conjugation using an Applied Biosystems/MDS SCIEX 5800 MALDI TOF/TOF analyzer (Foster City, Calif.) and TOF/TOF 5800 System Software (SCIEX, Concord, Ontario, Canada). Each sample was assigned a spot and mixed with a saturated sinapinic acid matrix on a 384 opti-tof 123×81 mm Rev A plate. Data were acquired in the linear high mass positive acquisition mode. The resulting haptenization ratio (number of haptens per mole of protein) of each conjugate was calculated as follows: [((MW_{conjugate}−MW_{carrier protein})/MWₕₐₚₜₑₙ]. Due to their large molecular weight, the haptenization ratio of sKLH conjugates could not be determined by MALDI-TOF and were consequently characterized for size by DLS on a Zetasizer (Malvern Panalytical Inc., United Kingdom) as previously described (Baruffaldi et al. Mol Pharm 15, 4947-4962 (2018)).

Activity of the fentanyl-based hapten at the Mu Opioid Receptor (MOR). To assess whether the F₁ hapten had any functional activity at the MOR, the F₁ hapten was tested in vitro in a calcium mobilization assay involving Chinese Hamster Ovary (CHO) cells co-expressing the human MOR and Gα16, a promiscuous G protein, as previously described (Raleigh et al. PLoS One 12, e0184876 (2017)). In these studies, the F₄ hapten and morphine were used as controls.

Active immunization. Mice were immunized IM with 60 μg of conjugates containing the F₁₋₇ hapten series or unconjugated sKLH, CRM₁ or CRM₂ as control. Conjugates were adsorbed on 30 g of alum (Alhydrogel85, Brenntag) and PBS to a final volume of 60 μL and delivered 30 μL per hindleg. Mice were immunized on days 0, 14, and 28. Rats were immunized IM with 60 μg of conjugates containing the F₁₋₇ hapten series or unconjugated carrier as control. Conjugates were adsorbed on 90 μg of alum and PBS to a final volume of 150 μL and delivered to one leg. Rats were immunized on days 0, 21, 42 and 63.

Antibody Analysis. Serum antibody analysis was performed via indirect ELISA after blood collection using facial vein sampling in mice or tail vein sampling in rats. 96-well plates were coated with 5 ng/well of the corresponding F₁₋₈-BSA conjugate or unconjugated BSA as a control. Conjugates were diluted in 50 mM Na₂CO₃, pH 9.6 (Sigma, C3041-100CAP) and blocked with 1% porcine gelatin. Immune sera samples were incubated on the plate and then washed and incubated with an HRP-conjugated goat anti-mouse IgG or goat anti-rat IgG (Jackson ImmunoResearch Laboratories, West Grove, Pa.) to assess hapten-specific serum IgG antibody levels as previously described (Laudenbach et al. J Immunol 194, 5926-5936 (2015)). For determination of affinity by competitive binding ELISA, 96-well plates were coated and blocked as described above, and fentanyl or fentanyl analogs were added to the wells with concentrations ranging from 1×10⁻⁴ M to 1×10⁻¹⁰ M. Immune sera were diluted to sub-saturating concentrations and incubated with competitor on the plate, and washed and incubated with HRP-conjugated antibody as above.

Effect of vaccine on opioid-induced analgesia via hot plate. To determine the efficacy of the vaccine to block opioid-induced analgesia, a hot plate test was performed. Rodents were allowed to acclimate to the testing environment for 1 hour prior to measuring baseline. Rodents were placed on a hot plate (Columbus Instruments, Columbus, Ohio) set to 54° C. and removed after displaying a lift or flick of the hindpaw or reaching the maximal cutoff of 60 seconds in mice or 30 seconds in rats to avoid thermal tissue damage. Mice were vaccinated on days 0, 14 and 28. On day 35, baselines were measured and mice were then given 0.05-0.1 mg/kg fentanyl, delivered subcutaneously. Hotplate responses were measured 30 minutes after injection. Rats were vaccinated on days 0, 21, 42 and 63. Starting at day 49, rats were tested repeatedly once a week. Drugs were given as a single subcutaneous dose. The initial drug tested was fentanyl at a dose of 0.075-1.0 mg/kg (week 1), followed by 0.008 mg/kg sufentanil (week 2), 0.5 mg/kg alfentanil and a final challenge of 0.1 mg/kg fentanyl (week 3). The latency to respond on the hotplate was measured at 15, 30, 45, and 60 minutes post-drug administration. At completion of the behavioral assessment (mice=31 minutes, and rats=61 minutes, which includes the 60 second maximal cutoff threshold), trunk blood and brain were collected for assessment of fentanyl concentrations. To test whether vaccination interfered with selected opioid agonists, and the reversal of their effects by naloxone, rats immunized with either CRM₁ or F₁-CRM₁ were challenged weekly with oxycodone (2.25 mg/kg, s.c.), heroin (0.9 mg/kg, s.c.), methadone (2.25 mg/kg, s.c.), and fentanyl (0.1 mg/kg, s.c., positive control) and hotplate responses were recorded at 30 minutes post-drug challenge. After receiving oxycodone or heroin, rats were given naloxone (0.1 mg/kg, s.c.) and their responses recorded for an additional 15 minutes. In both mouse and rat studies, data are displayed as maximal possible effect (MPE %) calculated as: (post-drug latency−baseline latency)/(maximal cutoff−baseline latency)×100.

Effect of vaccine against opioid-induced respiratory depression and bradycardia. To assess the efficacy of candidate vaccines against opioid-induced respiratory depression and bradycardia, oxygen saturation (SaO₂ %), breath rate (breaths per minute (brpm)), and heart rate (beats per minute (BPM)) were measured by oximetry before and after drug administration. Oximetry was measured using a MouseOx Plus pulse oximeter (Starr Life Sciences, Oakmont, Pa.). Rodents were allowed to acclimate to the testing environment for 1 hour prior to measuring baseline. After baseline recordings, rats were challenged s.c. repeatedly once a week with fentanyl (0.075-1.0 mg/kg), sufentanil (0.008 mg/kg), and alfentanil (0.5 mg/kg) as detailed in each experiment. In studies involving anesthetics, rats immunized with either CRM₁ or F₁-CRM₁ were first challenged with dexmedetomidine (0.25 mg/kg, s.c.), whose effects were reversed by atimpamizole (1 mg/kg, s.c.). In subsequent weeks, rats were challenged with ketamine (75 mg/kg, s.c.), propofol (100 mg/kg, s.c.), or isoflurane (2%, Drager Vapor 2000, Telford, Pa.). Measurements were obtained at 15, 30, 45, and 60 minutes post-drug administration.

Efficacy of vaccine against fentanyl intravenous self-administration (FSA). To provide proof of efficacy for therapeutic vaccination to reduce ongoing fentanyl intake, rats were trained in a fentanyl self-administration (FSA) assay using standard two-lever operant conditioning chambers (Med Associates, St. Albans, Vt.) prior to initiate the immunization regimen. Rats were implanted with jugular catheters and then trained to FSA (1 μg/kg/infusion) under a fixed-ratio (FR) 1 schedule during daily 120-min sessions (five days/week) according to a previously described standard protocol (Raleigh et al. PLoS One 9, el 15696 (2014), Pravetoni et al. PLoS One 9, e101807 (2014)). This unit dose was chosen because it maintains robust self-administration in rats and lies near the peak of the FSA dose-response curve (Lal et al. Commun Psychopharmacol 1, 207-212 (1977), Awasaki et al. Environ Toxicol Pharmacol 3, 115-122 (1997), Nishida et al. Eur J Pharmacol 166, 453-458 (1989), Stevenson et al. Pharmacol Biochem Behav 132, 49-55 (2015), Wager et al. ACS Chem Neurosci 8, 165-177 (2017)), providing a sensitive initial screen for vaccine efficacy. Responses on one lever produced fentanyl infusions at a rate of 0.1 ml/kg/sec, while responses on the other lever had no programmed consequence. After at least 10 sessions and when robust fentanyl intake was established, the FR was gradually increased to three over several sessions. After at least 10 sessions at FR 3 and once FSA stabilized (at least 20 infusions per session, greater than 2:1 ratio of active:inactive lever presses, and no significant trend across three consecutive sessions), rats were immunized i.m. with either CRM₁ (n=8) or F₁-CRM₁ (n=7) on days 0, 21, 42, and 63 while their FSA sessions continued. This course of injections allowed assessment of the effects of vaccination on maintenance of FSA at the training dose. Then, rats received a fifth vaccination on day 84 (3 weeks after the 4^{rd} vaccination) to allow testing the effects of vaccination on the FSA dose-response curve. For this phase, the fentanyl dose per infusion was reduced each week on Mondays using the following doses: 0.75, 0.50. 0.25, and 0 μg/kg/infusion. Three rats did not complete this phase because of catheter failure or death, resulting in a final sample size of seven CRM₁ rats and six F₁-CRM₁ rats.

Analysis of fentanyl concentration. Brain tissue was homogenized using Agilent ceramic beads with a Beadblaster 24 homogenizer (Benchmark Scientific, Sayreville, N.J.) and placed at −20° C. until extraction. Brain homogenate, serum and standards were processed in acetonitrile at 4° C., and then the supernatant was transferred, evaporated, and diluted in phosphate buffer. Samples were extracted using Bond Elut Plexa PCX extraction cartridges (Agilent, Santa Clara, Calif.), evaporated, and reconstituted in a solution of water, ammonium formate, and formic acid. Sample were injected onto a reversed phase Agilent (Santa Clara, Calif.) Zorbax Eclipse plus C18 column (2.1 mm×50 mm i.d., 1.8 μm), and then analyzed on an Agilent G6470A triple quadrupole LCMS/MS system including an Infinity 111290 G7116B Multicolumn Thermostat, G7120A High Speed Quad Pumps, G7267B Multisampler. Data acquisition and peak integration were analyzed using Mass Hunter software (Tokyo, Japan).

Selectivity of vaccine against anesthetic agents. To assess whether immunization against fentanyl does not interfere with anesthesia and rescue, Sprague Dawley rats were immunized i.m. with either CRM₁ or F₁-CRM₁ (n=6, each group) on days 0, 21, 42 and 63. From day 49, rats were challenged weekly with 2% inhaled isoflurane, 0.25 mg/kg dexmedetomidine (reversed by 1 mg/kg atipamezole), 75 mg/kg ketamine, 100 mg/kg propofol, or 0.1 mg/kg fentanyl as control. Anesthetic efficacy was measured by induction time reported as the latency of the loss of righting reflex, respiratory depression reported as percentage (%) of oxygen saturation and bradycardia reported as heart rate both measured by oximetry.

Statistical analysis. Fentanyl-specific serum antibody titers (LOG), fentanyl serum or brain concentrations, latency to respond in the hotplate nociception test, oxygen saturation (SaO₂), and heart rate (beats per minute, BPM) on single time points were compared using a one-way ANOVA paired with Dunnett's multiple comparison test, whereas comparison over multiple time points were analyzed by two-way ANOVA paired with Tukey's multiple comparison test. For the self-administration study, the mean number of infusions during the last three sessions at baseline and three weeks after the fourth vaccine injection was used to assess vaccine effects on maintenance of FSA. To assess vaccine effects on the FSA dose-response curve, the mean number of infusions during the last two sessions at each unit dose was used. FSA data were log transformed prior to statistical analysis due to non-normality of the data and heterogeneity of variance. The transformed data were analyzed using mixed-model ANOVA followed by Bonferroni's multiple comparison tests. All statistics were performed using Prism (version 8.0a.91; GraphPad, San Diego, Calif.).

### Results

Characterization of Biophysical Properties of Conjugate Vaccines. Biophysical properties of conjugate vaccines (including molecular weight (MW), measured by MALDI-TOF; estimated haptenation ratio; and whether the conjugate vaccine precipitates or aggregates) were characterized. Results are shown in Table 1A.

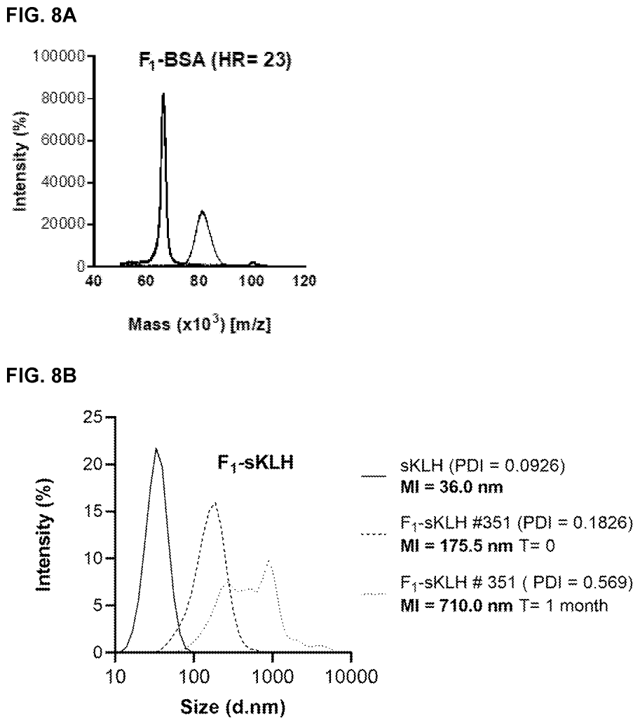

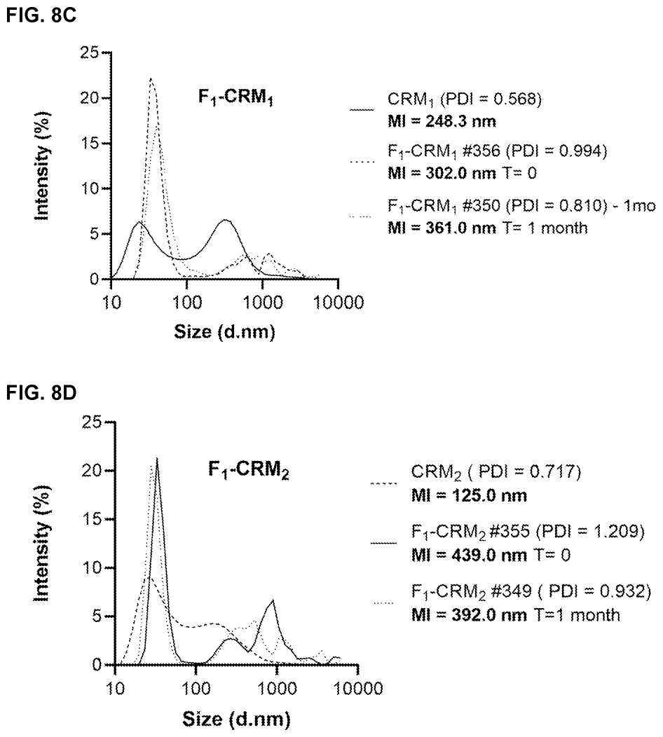

Additionally, FIG. 8 shows representative MALDI-TOF and DLS traces of F₁-BSA, F₁-sKLH, F₁-CRM and unconjugated carrier proteins (BSA, sKLH, CRM).

Testing of Conjugate Vaccines in Pre-Clinical Models. Mice and rats were immunized i.m., and then challenged with single or multiple s.c. doses of fentanyl and its analogs, as described above. FIG. 2 shows vaccine efficacy against fentanyl in mice. FIG. 3 shows vaccine efficacy against fentanyl in rats. FIG. 4 shows vaccine efficacy against sufentanil in rats. FIG. 5 shows efficacy of vaccines containing haptens F₄₋₆ against fentanyl and sufentanil in rats.

FIG. 6 shows active immunization reduced fentanyl intravenous self-administration (FSA) in rats. FIG. 7 shows immunization against fentanyl does not interfere with anesthesia and rescue.

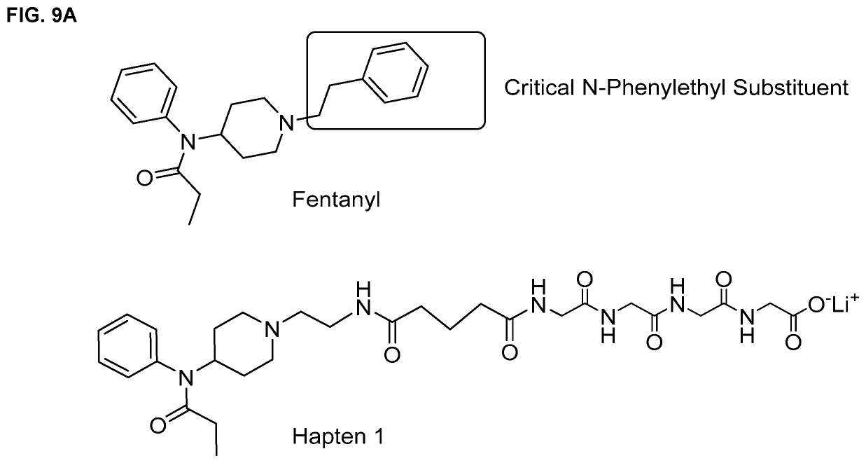

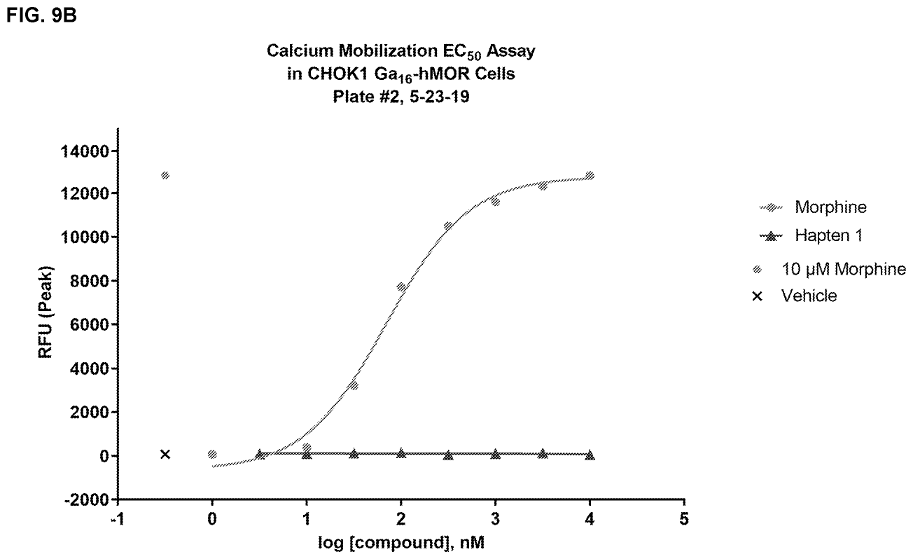

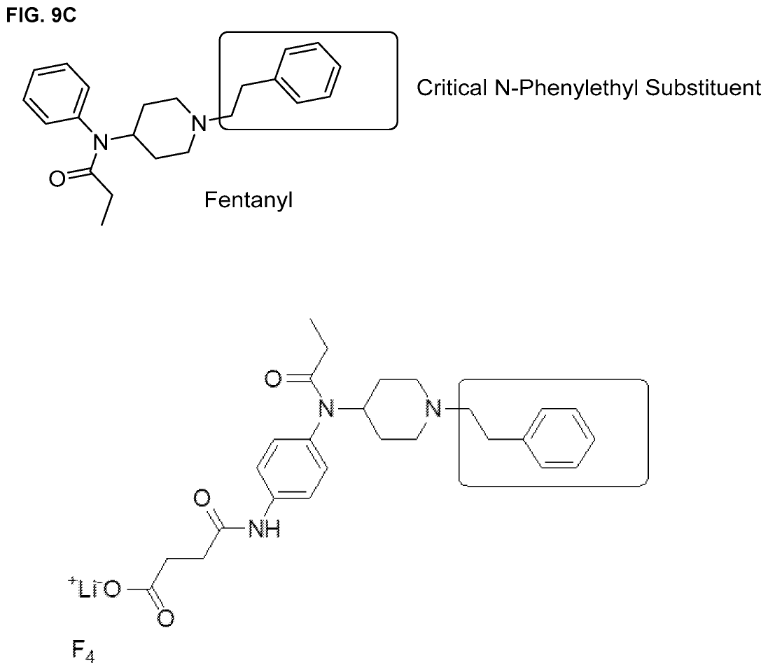

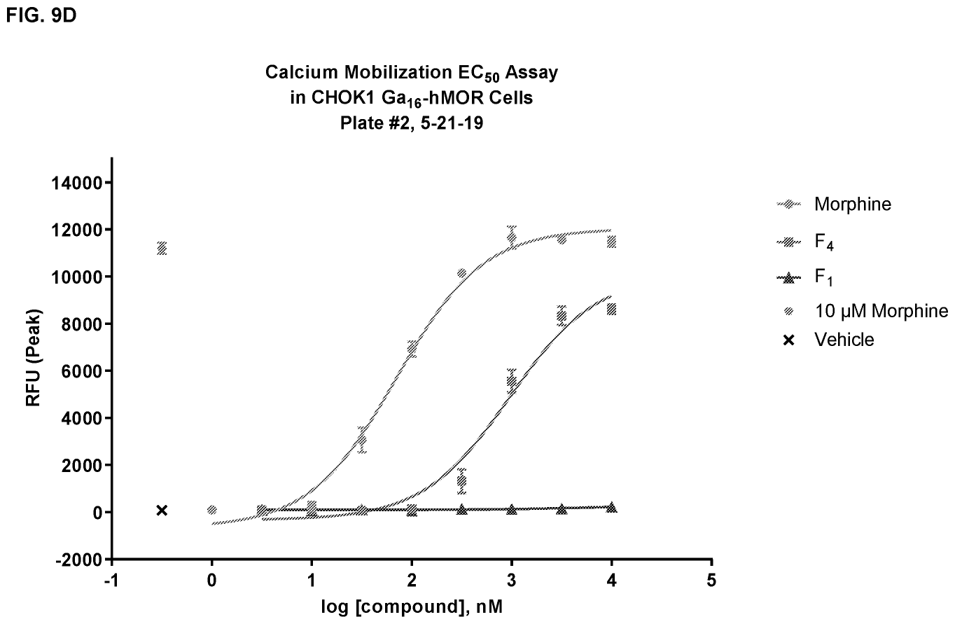

Activity of the fentanyl-based haptens at the Mu Opioid Receptor (MOR). Activity of the fentanyl-based hapten F₁ at the MOR was tested as described above. Results are shown in FIG. 9B and Table 4A. The F₁ hapten has no functional agonist activity at the MOR, most likely due to the extended peptidic linker and lack of an N-phenylethyl substituent.

Affinity of antibodies for fentanyl and its analogs. Sera from mice and rats immunized with conjugates containing fentanyl-based haptens was tested for the presence of antibodies that bind to fentanyl or its analogs. Analysis was performed by either competitive binding ELISA or BLI to determine either IC₅₀ or K_{d} for fentanyl or its analogs. Results are shown in Table 2.

Biomarkers predictive of vaccine efficacy against fentanyl and other opioids. Sera from mice and rats immunized with conjugates containing fentanyl-based haptens was tested for the presence of biomarkers predictive of vaccine efficacy against fentanyl and other opioids including opioid- and hapten-specific B cells, IL-4 modulation, IL-13 modulation, IL-2 modulation, PD-L1 modulation, IL-6 modulation, ICOSb, Fc gamma (γ) receptor I-IV, and Fcγ neonatal receptor. Results are shown in Table 3.

TABLE 1A
Characterization of conjugates containing the F₁₋₇ hapten series.

| | MW | Estimated HR | |
|---|---|---|---|
| | (MALDI- | (Haptenation | Precipitate/ |
| Conjugate | TOF) | Ratio) | Aggregate |
| F₁-BSA | 81297.47 | 23 | No |
| F₁-sKLH | N/A | N/A | Yes |
| F₁-CRM₁ | 70049.77 | 18, 17 | Slightly |
| F₁-CRM₂ | 66578.75 | 12, 17 | Slightly |
| F₂-BSA | 78185.07 | 23 | No |
| F₂-sKLH | N/A | N/A | No |
| F₃-BSA | 70713.41 | 9 | No |
| F₃-sKLH | N/A | N/A | No |
| F₃-CRM₂ | 61786.37 | 6 | Yes |
| F₄-BSA | 69473.86 | 7 | No |
| F₄-sKLH | N/A | N/A | No |
| F₅-BSA | 74246.50 | 17 | No |
| F₅-sKLH | N/A | N/A | Yes |
| F₅-CRM₂ | 63243.98 | 10 | Yes |
| F₆-BSA | 74060.00 | 17 | No |
| F₆-sKLH | N/A | N/A | N/A |
| F₆-CRM₂ | 62145.96 | 7 | Slightly |
| F₇-BSA | 81184.18 | 30 | No |
| F₇-sKLH | N/A | N/A | No |

F₁₋₇ haptens were conjugated to subunit KLH (sKLH), and either CRM₁ (E. coli expressed CRM from Fina Biosolutions) or CRM₂ (PFEnex) depending on product availability.

TABLE 1B
Characterization of conjugates containing the F₈₋₁₃ hapten series.

| | MW | Calculated | |
|---|---|---|---|
| | (MALDI- | Haptenization | Precipitate/ |
| Conjugate | TOF) | Ratio | Aggregate |
| F₈-BSA | 79701.0703 | 26.8 | No |
| F₈-CRM₂ | 58897.7148 | 14 | Yes |
| F₉ₐ-BSA | 72038.3984 | 16.2 | No |
| F₉ₐ-CRM₁ | 60394.3711 | 4.2 | No |
| F_{9b}-BSA | 69446.2891 | 8.0 | No |
| F_{9b}-CRM₁ | 62143.4883 | 8.5 | No |
| F₁₀-BSA | 82099.9766 | 25.7 | No |
| F₁₀-CRM₁ | 59055.0742 | 16 | No |
| F₁₁-BSA | 71073.9453 | 11.5 | No |
| F₁₁-CRM₂ | 62215.5859 | 9.8 | No |
| F₁₂-BSA | 85560.8281 | 30.4 | No |
| F₁₂-CRM₂ | 67100.4297 | 12.9 | Yes |
| F₁₃-BSA | 86100.7969 | 29 | No |
| F₁₃-CRM₂ | 69260.0469 | 16.3 | No |

F₈₋₁₃ haptens were conjugated to CRM₁ (E. coli expressed CRM, Fina Biosolutions) or CRM₂ (PFEnex), depending on product availability.

TABLE 2
Affinity of antibodies for fentanyl and its analogs, and off target opioids.
Sera from A) mice and B) rats immunized with conjugates containing fentanyl-
based haptens conjugated to either sKLH, EcoCRM (designated as CRM₁), or
CRM₁₉₇ (designated as CRM₂). C) lack of binding of off target opioids
used in treatment. Analysis was performed by either competitive
binding enzyme-linked immunosorbent assay (ELISA) or biolayer
interferometry (BLI) to determine either IC₅₀ or K_{d} for
fentanyl, its analogs, or off target opioids.
A. Mouse

| Vaccine Formulation | ID | Coating | Fentanyl IC50 (nM) |
|---|---|---|---|
| F₁-sKLH/alum/IM | 6501, 7699 | F₁-BSA | 204.4, 61.8 |
| F₁-sKLH/alum/IM | 6501, 7699 | F₂-BSA | 155.3, 598.6 |
| F₁-sKLH/alum/IM | 6501, 7699 | F₃-BSA | 11.8, 5.3 |
| F₁-sKLH/alum/IM | CER003 | F₁-BSA | 45.8, 3.30, 22.7, |
| | | | 15.0, 80.6, 68.5 |
| F₂-sKLH/alum/IM | n/a | F₂-BSA | 282.9 |
| F₂-CRM₁/alum/IM | n/a | F₂-BSA | 891.2 |
| F₃-sKLH/alum/IM | n/a | F₃-BSA | 140.3 |
| F₃-CRM₁/alum/IM | n/a | F₃-BSA | 67.9 |
| F₄-sKLH/alum/IM | CER003 | F₁-BSA | >100 μM (undetectable) |
| F₅-sKLH/alum/IM | CER003 | F₁-BSA | 14.1, 57.5 |
| F₆-CRM₁/alum/IM | CER003 | F₁-BSA | 5.84, 14.3 |

B. Rat

| | | Fentanyl | Sufentanil | Alfentanil | Remifentanil |
|---|---|---|---|---|---|
| Vaccine Formulation | Coating | IC₅₀ (nM) | IC₅₀ (μM) | IC₅₀ (μM) | IC₅₀ (μM) |
| F₁-sKLH/alum/IM | F₁-BSA | 14.24 | 17.78 | 60.23 | 64.72 |
| F₁-CRM₁/alum/IM | F₁-BSA | 530.25 | 14.60 | 100 | >100 |
| F₁-CRM₂/alum/IM | F₁-BSA | 113.21 | 65.13 | 66.55 | >100 |
| F₂-sKLH/alum/IM | F₂-BSA | 24.79 | 14.68 | 100 | >100 |
| F₃-sKLH/alum/IM | F₃-BSA | 69.58 | 10.55 | 100 | >100 |
| F₁-sKLH/alum/IM | F₁-BSA | 9.14, 91.5 | >100 μM | >100 μM | >100 μM |
| F₄-sKLH/alum/IM | F₁-BSA | >100 μM | >100 μM | >100 μM | >100 μM |
| F₅-sKLH/alum/IM | F₁-BSA | >100 μM | >100 μM | >100 μM | >100 μM |
| F₆-CRM₂/alum/IM | F₁-BSA | >100 μM | >100 μM | >100 μM | >100 μM |

C. Off-target opioids

| | | Buprenorphine | Naloxone | Naltrexone | Methadone |
|---|---|---|---|---|---|
| Vaccine Formulation | Coating | IC₅₀ (mM)* | IC₅₀ (mM) | IC₅₀ (mM) | IC₅₀ (mM) |
| F₁-sKLH | F₁-BSA | >0.3 | >10 | >10 | >10 |
| F₂-sKLH | F₁-BSA | >0.3 | 0.1, 1 | 1, >10 | >10, 1.1 |
| F₃-sKLH | F₁-BSA | >0.3 | 9.2, >10 | >10 | >10 |
| F₄-sKLH | F₁-BSA | >0.3 | >10 | >10 | >10 |
| F₅-sKLH | F₁-BSA | >0.3 | >10 | >10 | >16 |
| F₆-CRM₂ | F₁-BSA | >0.3 | >10 | 1.9, 2 | >10 |

Polyclonal sera from rats immunized with the series of conjugates containing F₁₋₈ hapten were analyzed by competitive binding ELISA, and when possible compared to a monoclonal antibody (mAb) isolated from mice immunized with F₁-sKLH. The affinity of the anti-F₁ mAb for fentanyl was verified by BLI and reported as a K_{d} of 1.592 nM and 0.5275 nM.
*The maximal concentration of buprenorphine tested was 0.3 mM because of the commercially available drug stock concentration. The maximal concentrations of naloxone, naltrexone, and methadone were 10 mM.

TABLE 3
Biomarkers predictive of vaccine efficacy
against fentanyl and other opioids

| Biomarker | Antibodies | Efficacy |
|---|---|---|
| Opioid- and hapten-specific B cells^{a} | ↑ | ↑ |
| Sex (female) | ↑ | ↑ |
| Environment & Microbiome | No Effect | ? |
| IL-4 modulation^{b} | ↑ | ↑ |
| IL-13 modulation | ↑ | ↑ |
| IL-2 modulation^{b} | No Effect | No Effect |
| PD-L1 modulation^{b} | ↓ | ↓ |
| IL-6 modulation | No Effect | No Effect |
| ICOS^{b} | ↓ | ↓ |
| Fc gamma (γ) receptor I-IV | No Effect | ↑ |
| Fcγ neonatal receptor | ↓ | ↓ |

^{a}(Laudenbach et al. J Immunol 194, 5926-5936 (2015), Laudenbach et al. Sci Rep 8, 5508 (2018), Laudenbach et al. Vaccine 33, 6332-6339 (2015), Taylor et al. J Immunol Methods 405, 74-86 (2014))
^{b}(Laudenbach et al. Sci Rep 8, 5508 (2018))

TABLE 4A

| | Morphine | Hapten 1 |
|---|---|---|
| log(agonist) vs. | | Ambiguous |
| response (three parameters) | | |
| Best-fit values | | |
| Bottom | −667.3 | 115.1 |
| Top | 12769 | ~−18428 |
| LogEC50 | 1.846 | ~6.651 |
| EC50 | 70.16 | ~4472958 |
| Span | 13436 | ~−18543 |

TABLE 4B

| | Morphine | F₄ | F₁ |
|---|---|---|---|
| log(agonist) vs. | | | |
| response (three parameters) | | | |
| Best-fit values | | | |
| Bottom | −674.8 | −339.7 | 93.18 |
| Top | 12044 | 10088 | 273.1 |
| LogEC50 | 1.846 | 2.980 | 3.641 |
| EC50 | 70.14 | 954.7 | 4376 |
| Span | 12719 | 10428 | 179.9 |
| % Morphine Emax | | 82% | X |

### Example 2—Synthesis of the F₈ Hapten

### Synthesis of the F₈ Hapten.

The F₈ hapten was synthesized as depicted in Step (a) to Step (g) of FIG. 1I. In FIG. 1I, the reagents and conditions used were as follows and as further described below: step a) N-Benzyl-4-piperidone, acetic acid (AcOH), Na(OAc)₃BH, DCM:THF, 0° C. to room temperature, 14 hours; step b) propionic anhydride, diisopropylethylamine (DIPEA), dichloromethane (DCM), 0° C. to room temperature, 20 hours; step c) ammonium formate, Pd (10% on C), MeOH, room temperature, 3 hours; step d) 1-(2-Bromoethyl)-4-ethyl-1,4-dihydro-5H-tetrazol-5-one, Na₂CO₃, 4-Methyl-2-pentanone, reflux, 4 hours; step e) HCl (4 M in dioxane), DCM, 0° C. to room temperature, 20 hours; step f) 4-nitrophenyl chloroformate, DIPEA, glycine methyl ester, tetrahydrofuran (THF), 0° C. to room temperature, 2 hours; step g) LiOH.H₂O, THF/MeOH/H₂O, 36 hours.

### tert-Butyl (4-((1-benzylpiperidin-4-yl)amino)phenyl)carbamate (1)

A solution of N-Boc-p-phenylenediamine (2.75 g, 13.21 mmol) in DCM:THF (100 mL, 1:1, v/v) was cooled to 0° C. and acetic acid (0.76 mL, 13.21 mmol) was added dropwise to the above solution. After that, N-Benzyl-4-piperidone (2.50 g, 13.21 mmol) was added, followed by sodium triacetoxyborohydride (4.20 g, 19.82 mmol) in three portions at 0° C. The reaction was warmed and stirred at room temperature for 14 hours. Upon completion, the reaction was quenched with saturated aqueous (aq.) NaHCO₃ (30 mL). The organic layer was separated, and the aqueous layer was extracted with DCM (3×30 mL). The combined organic layers were washed with brine (3×30 mL), and dried (Na₂SO₄). The solvent was removed under reduced pressure and the residue was subjected to chromatography on silica gel using 0-50% CMA80 in DCM to furnish amine 1 (4.58 g, 91%) as a yellow solid. (CMA80 is a mixture of 80% chloroform, 18% methanol, and 2% concentrated ammonium hydroxide.) ¹H NMR (300 MHz, CDCl₃) δ 7.35-7.21 (m, 5H), 7.11 (d, J=8.3 Hz, 2H), 6.61-6.44 (m, 2H), 6.31 (s, 1H), 3.51 (s, 2H), 3.42-3.13 (m, 2H), 2.89-2.75 (m, 2H), 2.12 (td, J=11.6, 2.3 Hz, 2H), 2.05-1.94 (m, 2H), 1.52-1.34 (m, 11H); ¹³C NMR (75 MHz, CDCl₃) δ 153.5, 143.6, 138.4, 129.1, 128.6, 128.2, 127.0, 121.4, 113.9, 79.9, 63.2, 52.4, 50.5, 32.6, 28.4; MS (ESI) m/z: calculated for C₂₃H₃₁N₃O₂ 381.51, found 382.4 [M+H]⁺.

### tert-Butyl (4-(N-(1-benzylpiperidin-4-yl)propionamido)phenyl)carbamate (2)

To a solution of amine 1 (4.30 g, 11.27 mmol) in dry DCM (60 mL), DIPEA (3.85 mL, 22.54 mmol) and propionic anhydride (5.75 mL, 45.08 mmol) was added at 0° C. The reaction which resulted was stirred for 20 hours at room temperature. At this point, the reaction was cooled to 0° C. and quenched with saturated aq. NaHCO₃ (40 mL). The layers were separated, and the aqueous layer was extracted with additional DCM (3×50 mL). The combined organic layers were washed with brine (3×50 mL), dried (Na₂SO₄), and concentrated under reduced pressure to give a yellowish residue. The residue was subjected to chromatography on silica gel using 0-50% CMA80 in DCM to furnish amide 2 (4.58 g, 93%) as a yellow solid. ¹H NMR (300 MHz, deuterated chloroform (CDCl₃) δ 7.65 (s, 1H), 7.40 (d, J=8.6 Hz, 2H), 7.34-7.14 (m, 5H), 6.92 (d, J=8.5 Hz, 2H), 4.70-4.53 (m, 1H), 3.53 (s, 2H), 3.06-2.91 (m, 2H), 2.31-2.10 (m, 2H), 1.94 (q, J=7.4 Hz, 2H), 1.80-1.67 (m, 2H), 1.57-1.35 (m, 11H), 0.99 (t, J=7.4 Hz, 3H); ¹³C NMR (75 MHz, CDCl₃) δ 174.0, 152.9, 138.9, 136.6, 133.0, 130.5, 129.5, 128.2, 127.4, 118.9, 80.6, 62.3, 52.5, 51.9, 29.8, 28.4, 28.3, 9.6; MS (ESI) m/z: calculated for C₂₆H₃₅N₃O₃ 437.57, found 438.6 [M+H]⁺.

### tert-Butyl (4-(N-(piperidin-4-yl)propionamido)phenyl)carbamate (3)

To a solution of benzylamine 2 (4.58 g, 10.47 mmol) in MeOH (60 mL), ammonium formate (3.30 g, 52.33 mmol) and Pd (2.23 g, 2.09 mmol, 10% on carbon were added. The resulting reaction was stirred at room temperature for 3 hours. The reaction mixture was filtered through Celite, and the filtrate was concentrated to dryness. The residue was re-dissolved in DCM (50 mL), washed with saturated aq. NaHCO₃ (30 mL), brine (3×50 mL), dried (Na₂SO₄), and concentrated in vacuo to provide amine 3 (3.12 g, 86%) as a white solid. ¹H NMR (300 MHz, CDCl₃) δ 7.41 (d, J=8.7 Hz, 2H), 7.03-6.90 (m, 3H), 4.80-4.62 (m, 1H), 3.15-2.98 (m, 2H), 2.85-2.61 (m, 3H), 1.93 (q, J=7.4 Hz, 2H), 1.84-1.71 (m, 2H), 1.53 (s, 9H), 1.34-1.16 (m, 2H), 1.00 (t, J=7.4 Hz, 3H); ¹³C NMR (75 MHz, CDCl₃) δ 173.7, 152.6, 138.6, 133.5, 130.8, 118.9, 80.9, 52.2, 45.8, 31.6, 28.4, 28.3, 9.6; MS (ESI) m/z: calculated for C₁₉H₂₉N₃O₃ 347.45, found 348.4 [M+H]⁺.

### tert-Butyl (4-(N-(1-(2-(4-ethyl-5-oxo-4,5-dihydro-1H-tetrazol-1-yl)ethyl)piperidin-4-yl)propion-amido)phenyl)carbamate (4)

A suspension of amine 3 (0.80 g, 2.30 mmol), 1-(2-Bromoethyl)-4-ethyl-1,4-dihydro-5H-tetrazol-5-one (0.29 mL, 2.30 mmol) and Na₂CO₃ (0.49 g, 4.60 mmol) in 4-Methyl-2-pentanone (15 mL) was refluxed for 4 hours. After that, the solution was cooled to room temperature and diluted with EtOAc (20 mL). The solution was then washed with H₂O (2×30 mL), brine (2×20 mL), dried (Na₂SO₄), and concentrated under reduced pressure. The residue was subjected to chromatography on silica gel using 0-50% CMA80 in EtOAc to furnish tetrazolone 4 (0.89 g, 80%) as a white solid. ¹H NMR (300 MHz, CDCl₃) δ 7.41 (d, J=8.6 Hz, 2H), 6.96 (d, J=8.6 Hz, 2H), 6.81 (s, 1H), 4.67-4.52 (m, 1H), 4.07-3.86 (m, 4H), 3.04-2.83 (m, 2H), 2.79-2.65 (m, 2H), 2.27-2.11 (m, 2H), 2.01-1.84 (m, 3H), 1.78-1.67 (m, 2H), 1.53 (s, 9H), 1.40-1.30 (m, 4H), 0.99 (t, J=7.4 Hz, 3H); ¹³C NMR (75 MHz, CDCl₃) δ 173.8, 152.6, 150.7, 138.5, 133.4, 130.8, 118.9, 80.9, 55.5, 52.9, 52.0, 42.4, 40.0, 30.4, 28.4, 28.3, 13.7, 9.6; MS (ESI) m/z: calculated for C₂₄H₃₇N₇O₄ 487.60, found 488.6 [M+H]⁺.

### N-(4-aminophenyl)-N-(1-(2-(4-ethyl-5-oxo-4,5-dihydro-1H-tetrazol-1-yl)ethyl)piperidin-4-yl)propionamide (5)

To an ice-cold solution of carbamate 4 (0.89 g, 1.83 mmol) in dry DCM (20 mL), HCl (4.58 mL, 4 M solution in dioxane) was added. The reaction, which resulted was stirred at room temperature for 20 hours. The solvent was removed under reduced pressure and the residue was subjected to chromatography on silica gel using 0-100% CMA80 in DCM to furnish amine 5 (0.59 g, 84%) as a white solid. ¹H NMR (300 MHz, CDCl₃) δ 6.88-6.71 (m, 2H), 6.70-6.56 (m, 2H), 4.66-4.50 (m, 1H), 4.05-3.89 (m, 4H), 3.80 (br s, 2H), 2.99-2.85 (m, 2H), 2.71 (t, J=6.9 Hz, 2H), 2.27-2.10 (m, 2H), 1.95 (q, J=7.5 Hz, 2H), 1.76-1.64 (m, 2H), 1.41-1.23 (m, 5H), 0.99 (t, J=7.5 Hz, 3H); ¹³C NMR (75 MHz, CDCl₃) δ 174.2, 150.7, 146.3, 131.0, 129.3, 115.1, 55.6, 53.0, 51.9, 42.4, 40.0, 30.4, 28.3, 13.7, 9.6; MS (ESI) m/z: calculated for C₁₉H₂₉N₇O₂ 387.48, found 388.4 [M+H]⁺.

### Methyl 2-(3-(4-(N-(1-(2-(4-ethyl-5-oxo-4,5-dihydro-1H-tetrazol-1-yl)ethyl)piperidin-4-yl)propionamido)phenyl)ureido)acetate (6)

To a suspension of amine 5 (376 mg, 0.97 mmol) in THF (6 mL), DIPEA (0.42 mL, 2.43 mmol) was added. The reaction was then cooled to 0° C., and a solution of 4-nitrophenyl chloroformate (0.24 g, 1.16 mmol) in THF (3 mL) was added dropwise. After that, the reaction was stirred at 0° C. for 30 min. At this point, a solution of glycine methyl ester (0.17 g, 1.94 mmol) and DIPEA (0.42 mL, 2.43 mmol) in THF (10 mL) was added to the above reaction. The reaction was then stirred for 30 min at 0° C. and an additional 1.5 h at room temperature. The reaction was then quenched with MeOH (5 mL) and concentrated to dryness under reduced pressure. The residue was subjected to chromatography on silica gel using 0-100% CMA80 in EtOAc to furnish urea 6 (314.5 mg, 64%) as a white solid. ¹H NMR (300 MHz, CDCl₃) δ 8.04-7.83 (m, 1H), 7.42 (d, J=8.7 Hz, 2H), 6.95 (d, J=8.5 Hz, 2H), 6.14-5.95 (m, 1H), 4.73-4.47 (m, 1H), 4.08 (d, J=5.2 Hz, 2H), 4.04-3.90 (m, 4H), 3.76 (s, 3H), 2.92 (d, J=10.9 Hz, 2H), 2.71 (t, J=6.7 Hz, 2H), 2.17 (t, J=11.3 Hz, 2H), 1.95 (q, J=7.4 Hz, 2H), 1.73 (d, J=11.3 Hz, 2H), 1.43-1.22 (m, 5H), 1.00 (t, J=7.4 Hz, 3H); ¹³C NMR (75 MHz, CDCl₃) δ 174.3, 171.6, 155.4, 151.0, 139.4, 133.4, 130.9, 120.0, 55.7, 53.1, 52.5, 52.4, 42.6, 42.1, 40.3, 30.6, 28.7, 13.9, 9.8; MS (ESI) m/z: calculated for C₂₃H₃₄N₈O₅ 502.57, found 503.6 [M+H]⁺. HPLC (280 nm) t_{R}=10.10 min.

### Lithium 2-(3-(4-(N-(1-(2-(4-ethyl-5-oxo-4,5-dihydro-1H-tetrazol-1-yl)ethyl)piperidin-4-yl)propionamido)phenyl)ureido)acetate (F₈)

To a solution of the ester 6 (314 mg, 0.62 mmol) in THF/MeOH/H₂O (5 mL, 1:1:0.5, v/v/v), LiOH.H₂O (33 mg, 0.78 mmol) was added and the reaction, which resulted, was stirred at room temperature for 36 hours. After that, the solvent was removed under nitrogen flow to provide lithium salt F₈ (310 mg, quant.) as a white solid. ¹H NMR (300 MHz, DMSO-d₆) δ 10.86 (br s, 1H), 7.86 (br s, 1H), 7.61 (d, J=8.5 Hz, 2H), 6.93 (d, J=8.6 Hz, 2H), 4.45-4.28 (m, 1H), 3.93 (t, J=6.1 Hz, 2H), 3.84 (q, J=7.2 Hz, 2H), 3.71-3.61 (m, 2H), 2.83 (d, J=10.3 Hz, 2H), 2.58 (t, J=6.2 Hz, 2H), 2.02 (t, J=11.2 Hz, 2H), 1.84 (q, J=7.4 Hz, 2H), 1.60 (d, J=9.7 Hz, 2H), 1.27-1.05 (m, 5H), 0.86 (t, J=7.4 Hz, 3H); ¹³C NMR (75 MHz, DMSO-d₆) δ 173.3, 172.3, 155.4, 150.1, 141.7, 130.5, 129.9, 117.9, 54.9, 52.2, 51.5, 45.0, 41.7, 39.4, 30.0, 27.6, 13.4, 9.5; MS (ESI) m/z: calculated for C₂₂H₃₂N₈O₅ 488.54, found 489.4 [M+H]⁺. HPLC (220 nm) t_{R}=9.35 min.

### Example 3A—Synthesis of the F₉ₐ and F_{9b} Haptens

Synthesis of common carboxylic acid intermediate. A mixture of norfentanyl (3.0 g, 12.9 mmol, 1.0 equiv) in 18 mL of anhydrous CH₃CN was treated with tert-butyl acrylate (2.4 mL, 16.1 mmol, 1.25 equiv) via syringe at ambient temperature. The reaction was maintained for 24 hours. TLC analysis (5% MeOH/CH₂Cl₂) showed the reaction was mostly complete. The resulting solution was concentrated to afford a viscous, yellow oil.

The crude product was purified by filtering through a plug of SiO₂ (150 mL fritted glass funnel) eluting with CH₂Cl₂ (250 mL) then 5% MeOH/CH₂Cl₂ (400 mL) to afford 4.2 g (90%) of a white solid. MS: m/z 305.16 [M-^{t}Bu+2H]⁺.

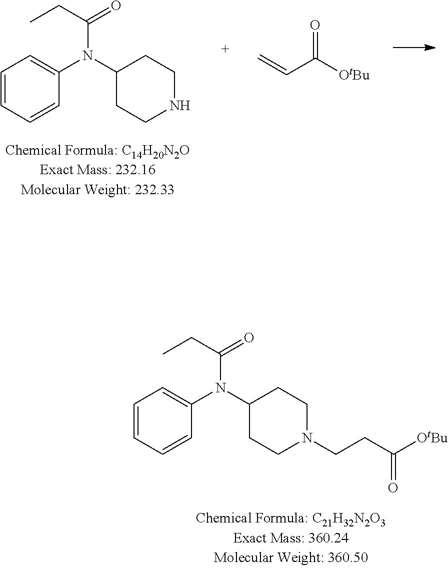

tert-Butyl acrylate (0.863 g, 2.39 mmol, 1.0 equiv) was treated with TFA (11 mL, 60 equiv) at ambient temperature. The reaction was maintained for 1.5 hours. LC-MS analysis showed that the reaction was complete. The reaction was concentrated to afford a viscous, yellow oil. Trituration from dry diethyl ether (Et₂O) resulted in the formation of a white precipitate. The solid was filtered and washed with Et₂O then dried under reduced pressure to afford 960 mg (96%) of a white solid. MS: m/z 305.12 [M+H]⁺.

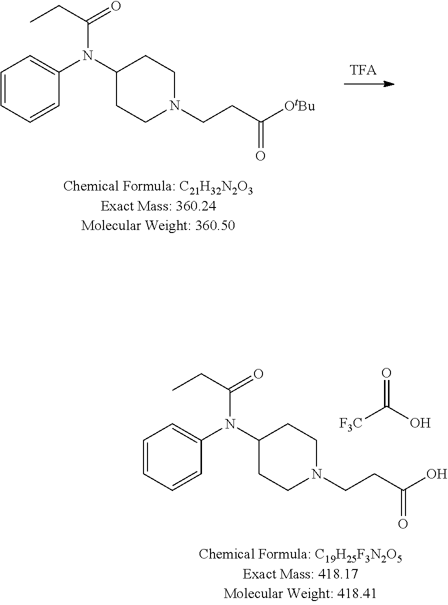

Synthesis of F₉ₐ (amino fentanyl) hapten. To a 0° C. suspension of acid (0.300 g, 0.717 mmol, 1.0 equiv) in 7 mL of anhydrous CH₂Cl₂ was added N-Boc ethylenediamine (0.170 mL, 1.08 mmol, 1.5 equiv) via pipette. The suspension became a clear solution. EDC (0.344 g, 1.79 mmol, 2.5 equiv) and dimethylaminopyridine (DMAP) (9 mg, 0.0717 mmol, 10 mol %) were added and the reaction was maintained at ambient temperature for 22 hours. LC-MS analysis showed the reaction was complete. The reaction mixture was diluted with CH₂Cl₂ (60 mL) and washed with 1 M NaOH (2×40 mL), H₂O (2×40 mL), and brine. The organics were dried over anhydrous MgSO₄, filtered, and concentrated. Purification by flash chromatography on SiO₂ (45 g, 8% MeOH/CH₂Cl₂) afforded 285 mg (89%) of a white foam. MS: m/z 447.27 [M+H]⁺, 469.25 [M+Na]⁺.

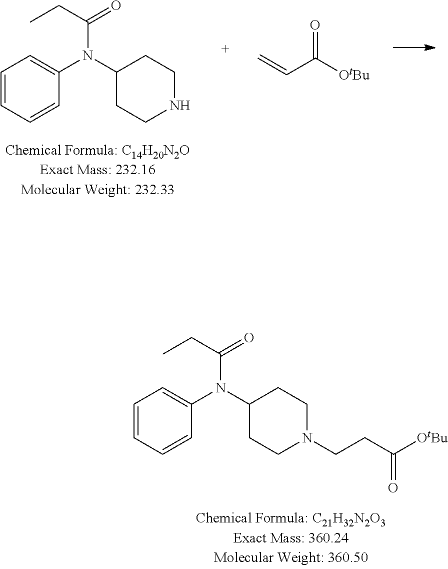

tert-Butyl ester (0.863 g, 2.39 mmol, 1.0 equiv) was treated with TFA (11 mL, 60 equiv) at ambient temperature. The reaction was maintained for 1.5 hours. LC-MS analysis showed that the reaction was complete. The reaction was concentrated to afford a viscous, yellow oil. Trituration from dry Et₂O resulted in the formation of a white precipitate. The solid was filtered and washed with Et₂O then dried under reduced pressure to afford 960 mg (96%) of a white solid. MS: m/z 305.12 [M+H]⁺.

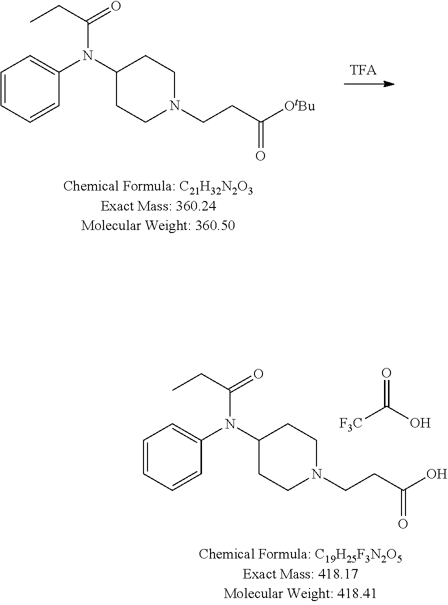

Synthesis of F_{9b} (carboxylic acid fentanyl) hapten. To a 0° C. suspension of carboxylic acid (0.300 g, 0.717 mmol, 1.0 equiv) in 7 mL of anhydrous CH₂Cl₂ was added β-alanine tert-butyl ester hydrochloride (0.195 g, 1.08 mmol, 1.5 equiv) followed by diisopropylethylamine (0.250 mL, 1.43 mmol, 2.0 equiv). The resulting clear solution was then treated with EDC (0.344 g, 1.79 mmol, 2.5 equiv) and DMAP (9 mg, 0.0717 mmol, 10 mol %). The reaction was maintained at ambient temperature for 20 hours. LC-MS analysis showed the reaction was complete. The mixture was diluted with CH₂Cl₂ (60 mL) then washed with 1 M NaOH (2×40 mL), H₂O (2×40 mL), and brine. The organics were dried over anhydrous MgSO₄, filtered, and concentrated. Purification by flash chromatography on SiO₂ (42 g, 5% MeOH/CH₂Cl₂) afforded 286 mg (92%) of a viscous, pale yellow oil. MS: m/z 432.23 [M+H]⁺.

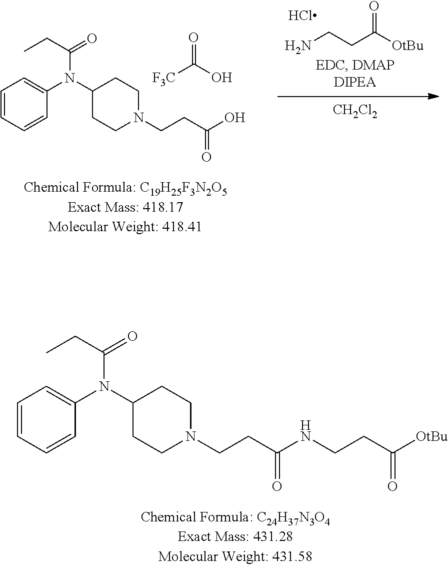

tert-Butyl ester (0.188 g, 0.436 mmol, 1.0 equiv) was treated with TFA (2 mL, 60 equiv) at ambient temperature. The reaction was maintained for 1.5 hours. LC-MS analysis showed that the reaction was complete. The reaction mixture was concentrated under reduced pressure to afford a viscous, yellow oil. Trituration from dry Et₂O afforded a sticky, white oil. The Et₂O was decanted and the residue was washed with Et₂O then dried under reduced pressure to yield 204 mg (96%) of a tacky, pale yellow residue (F_{9b}). MS: m/z 376.15 [M+H]⁺.

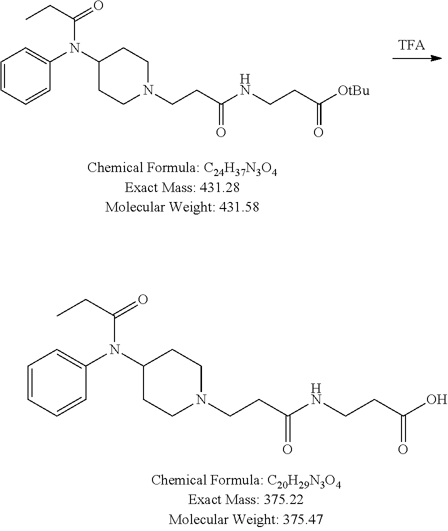 [SMILES: CCC(=O)N(c1ccccc1)C1CCN(CCC(=O)NCCC(=O)OC(C)(C)C)CC1 ; CCC(=O)N(c1ccccc1)C1CCN(CCC(=O)NCCC(=O)O)CC1]

### Example 3B—Alternate Synthesis of the F₉ₐ and F_{9b} Haptens

Synthesis of common carboxylic acid intermediate. A mixture of norfentanyl (3.0 g, 12.9 mmol, 1.0 equiv) in 18 mL of anhydrous CH₃CN was treated with tert-butyl acrylate (2.4 mL, 16.1 mmol, 1.25 equiv) via syringe at ambient temperature. The reaction was maintained for 24 hours. TLC analysis (5% MeOH/CH₂Cl₂) showed the reaction was mostly complete. The resulting solution was concentrated to afford a viscous, yellow oil.

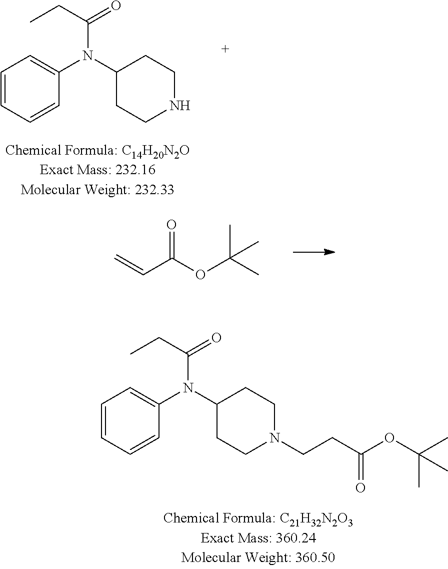

The crude product was purified by filtering through a plug of SiO₂ (150 mL fritted glass funnel) eluting with CH₂Cl₂ (250 mL) then 5% MeOH/CH₂Cl₂ (400 mL) to afford 4.2 g (90%) of a white solid.

tert-Butyl acrylate (0.500 g, 1.39 mmol, 1.0 equiv) was treated with TFA (6.4 mL, 60 equiv) at ambient temperature. The reaction was maintained for 1 hour. LC-MS analysis showed that the reaction was complete. The reaction was concentrated to afford a viscous, yellow oil. Trituration from dry Et₂O resulted in the formation of a white precipitate. The solid was filtered and washed with Et₂O then dried under reduced pressure to afford 545 mg (94%) of a white solid. F₉ₐ and F_{9b} were prepared from this intermediate.

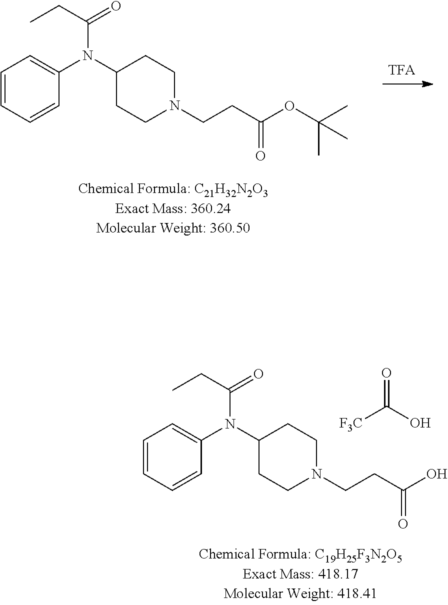

Synthesis of F₉ₐ (amino fentanyl) hapten. To a 0° C. suspension of acid (0.200 g, 0.478 mmol, 1.0 equiv) in 5 mL of anhydrous CH₂Cl₂ was added N-Boc ethylenediamine (0.115 mL, 0.717 mmol, 1.5 equiv) via pipette. The suspension became a clear solution. EDC (0.229 g, 1.20 mmol, 2.5 equiv) and DMAP (6 mg, 0.0478 mmol, 10 mol %) were added and the reaction was maintained at ambient temperature for 24 hours. LC-MS analysis showed the reaction was complete. The reaction mixture was diluted with CH₂Cl₂ (60 mL) and washed with 0.1 M NH₄Cl, 1 M NaOH, H₂O, and brine. The organics were dried over MgSO₄, filtered, and concentrated.

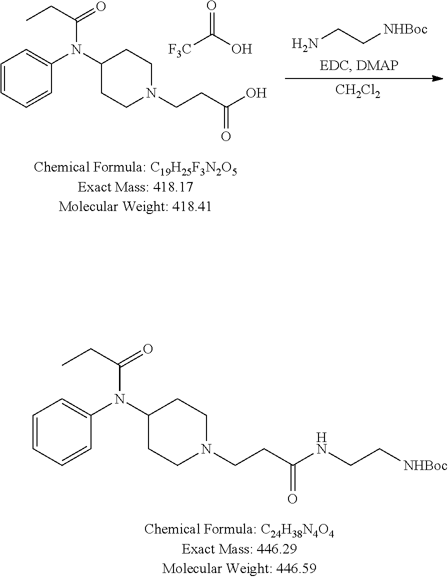

Purification by flash chromatography on SiO₂ (35 g, 9% MeOH/CH₂Cl₂) afforded 132 mg (62%) of a white solid.

Boc amine X was treated with 5.6 mL of 4 M HCl in dioxane at 0° C. The resulting reaction mixture was the removed from the ice bath and maintained at ambient temperature. After 1 hour, the reaction was concentrated under reduced pressure and triturated with anhydrous Et₂O. The resulting precipitate was filtered and dried to yield 83 mg (70%) of a tacky, off-white solid (F₉ₐ). MS: m/z 347.20 [M+H]⁺, 369.16 [M+Na]⁺.

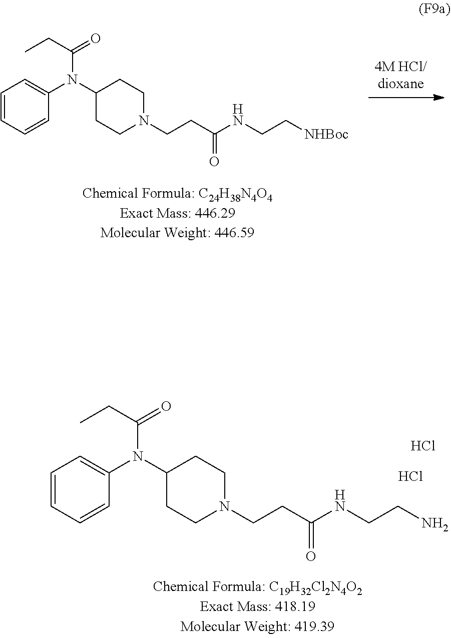 [SMILES: CCC(=O)N(c1ccccc1)C1CCN(CCC(=O)NCCNC(=O)OC(C)(C)C)CC1 ; CCC(=O)N(c1ccccc1)C1CCN(CCC(=O)NCCN)CC1 ; Cl ; Cl]

Synthesis of F_{9b} (carboxylic acid fentanyl) hapten. F_{9b} hapten was synthesized as described in Example 3A from the carboxylic acid intermediate made as described above.

### Example 4—Synthesis of Haptens F₁₀ to F₁₃

### Synthesis of the F₁₀ Hapten.

The F₁₀ hapten was synthesized as depicted in step (a) to step (h) of FIG. 1L. In FIG. 1L, the reagents and conditions used were as follows and as further described below: step a) N-Boc-2-aminoacetaldehyde, Na(OAc)₃BH, 1,2-DCE, 0° C. to room temperature, 4 hours; step b) PhNH₂, AcOH, Na(OAc)₃BH, DCM, 0° C. to room temperature, 14 hours; step c) Ac₂O, DIPEA, DCM, 0° C. to room temperature, 24 hours; step d) HCl (4 M in dioxane), DCM, 0° C. to room temperature, 3 hours; step e) Methyl glutaryl chloride, triethylamine (TEA), DCM, 0° C. to room temperature, 1 hour; step f) LiOH.H₂O, THF/MeOH/H₂O, 6 hours; g) gly₄OMe, benzotriazol-1-yloxytris(dimethylamino)phosphonium hexafluorophosphate (BOP), TEA, DMF, 0° C. to room temperature, 8 hours; step h) LiOH.H₂O, THF/MeOH/H₂O, 30 hours.

### tert-Butyl (2-(4-oxopiperidin-1-yl)ethyl)carbamate (1)

4-Piperidone monohydrate hydrochloride (5 g, 32.55 mmol) was dissolved in 1,2-DCE (50 mL). N-Boc-2-aminoacetaldehyde (6.2 g, 39.06 mmol) was dissolved in 1,2-DCE (10 mL) and added dropwise to the above solution at 0° C. After that, sodium triacetoxyborohydride (10.3 g, 48.82 mmol) was added to the above reaction in three portions at 0° C. The reaction was warmed to room temperature and stirred for 4 hours. After completion of the reaction, as indicated by LCMS, the reaction was quenched with saturated aq. NaHCO₃ (30 mL). The organic layer was separated, and the aqueous layer was extracted with DCM (3×50 mL). The combined organic layers were washed with brine (3×50 mL), and dried (Na₂SO₄). The solvent was removed under reduced pressure to furnish compound 1 as a yellowish residue (7.83 g, 99% crude yield). This material was used for the next transformation without purification. MS (ESI) m/z: calculated for C₁₂H₂₂N₂O₃ 242.31, found 243.2 (M+H)⁺.

### tert-Butyl (2-(4-(phenylamino)piperidin-1-yl)ethyl)carbamate (2)

A solution of aniline (2.9 mL, 32.31 mmol) in DCM (50 mL) was cooled to 0° C. Acetic acid (1.8 mL, 32.31 mmol) was added dropwise to the above solution at 0° C. After that, piperidone 1 (7.83 g, 32.31 mmol) dissolved in DCM (20 mL) was added slowly, followed by sodium triacetoxyborohydride (10.27 g, 48.47 mmol) in three portions at 0° C. The reaction was stirred at room temperature for 14 hours. The reaction was then quenched with saturated aq. NaHCO₃ (30 mL). The layers were separated, and the aqueous layer was extracted with additional DCM (3×30 mL). The combined organic layers were washed with brine (3×30 mL), dried (Na₂SO₄), and concentrated under reduced pressure. The residue was subjected to chromatography on silica gel using 0-100% CMA80 in DCM to furnish amine 2 (3.72 g, 36%) as a waxy white solid. ¹H NMR (300 MHz, CDCl₃) δ 7.18 (dd, J=8.4, 7.4 Hz, 2H), 6.70 (t, J=7.3 Hz, 1H), 6.61 (d, J=7.7 Hz, 2H), 5.08 (br s, 1H), 3.54 (br s, 1H), 3.41-3.08 (m, 3H), 2.88 (d, J=11.6 Hz, 2H), 2.50 (t, J=5.9 Hz, 2H), 2.19 (t, J=11.3 Hz, 2H), 2.12-1.95 (m, 2H), 1.62-1.32 (m, 11H); ¹³C NMR (75 MHz, CDCl₃) δ 155.9, 147.1, 129.3, 117.3, 113.3, 79.2, 57.2, 52.2, 49.8, 37.4, 32.4, 28.4; MS (ESI) m/z: calculated for C₁₈H₂₉N₃O₂ 319.44, found 320.4 (M+H)⁺.

### tert-Butyl (2-(4-(N-phenylacetamido)piperidin-1-yl)ethyl)carbamate (3)

To a solution of amine 2 (1.00 g, 3.13 mmol) in dry DCM (30 mL), DIPEA (1.1 mL, 6.26 mmol) and acetic anhydride (1.3 mL, 12.52 mmol) was added at 0° C. The reaction which resulted, was stirred at room temperature for 24 h. The reaction was cooled to 0° C. and quenched with saturated aq. NaHCO₃ (30 mL). The layers were separated, and the aqueous layer was extracted with additional DCM (3×30 mL). The combined organic layers were washed with brine (3×50 mL), dried (Na₂SO₄), and concentrated under reduced pressure to give a yellowish residue. The residue was subjected to chromatography on silica gel using 0-100% CMA80 in DCM to furnish acetamide 3 (1.10 g, 97%) as a yellowish oil. ¹H NMR (300 MHz, CDCl₃) δ 7.40-7.28 (m, 3H), 7.07-6.97 (m, 2H), 4.87 (br s, 1H), 4.64-4.48 (m, 1H), 3.14-3.00 (m, 2H), 2.81 (d, J=10.7 Hz, 2H), 2.32 (t, J=5.9 Hz, 2H), 2.05 (t, J=11.7 Hz, 2H), 1.76-1.64 (m, 5H), 1.37-1.27 (m, 11H. ¹³C NMR (75 MHz, CDCl₃) δ 170.1, 155.8, 139.4, 130.1, 129.3, 128.3, 79.0, 57.1, 52.9, 52.2, 37.4, 30.3, 28.4, 23.4; MS (ESI) m/z: calculated for C₂₀H₃₁N₃O₃ 361.48, found 362.4 (M+H)⁺.

### N-(1-(2-Aminoethyl)piperidin-4-yl)-N-phenylacetamide hydrochloride (4)

To an ice cold solution of carbamate 3 (0.08 g, 0.21 mmol) in dry DCM (6 mL), HCl (0.6 mL, 4 M solution in dioxane) was added. The reaction was stirred at room temperature for 3 hours. The solvent was removed under reduced pressure and excess HCl was removed by flash evaporation with DCM (3×10 mL) to provide the HCl salt of amine 4 (0.05 g, 76%) as a white solid. ¹H NMR (300 MHz, CD₃OD) δ 7.59-7.46 (m, 3H), 7.29 (d, J=6.9 Hz, 2H), 4.87-4.76 (m, 1H), 3.72 (d, J=11.7 Hz, 2H), 3.52-3.41 (m, 4H), 3.36-3.28 (m, 2H), 2.21-2.11 (m, 2H), 1.95-1.82 (m, 2H), 1.78 (s, 3H); ¹³C NMR (75 MHz, CD₃OD) δ 173.1, 139.9, 131.2, 131.1, 130.3, 54.5, 54.0, 51.3, 35.3, 28.9, 23.4; MS (ESI) m/z: calculated for C₁₅H₂₃N₃O 261.36, found 262.4 (M+H)⁺.

### Methyl 5-oxo-5-((2-(4-(N-phenylacetamido)piperidin-1-yl)ethyl)amino)pentanoate (5)

To a solution of amine.HCl 4 (0.05 g, 0.17 mmol) in dry DCM (5 mL), TEA (0.13 mL, 0.96 mmol) and glutaric acid monomethyl ester chloride (0.03 mL, 0.24 mmol) was added at 0° C. The reaction was then stirred at room temperature for 1 hour. After the completion of the reaction, as indicated by TLC, the reaction was quenched with cold aq. NaHCO₃ (20 mL). The layers were separated, and the aqueous layer was extracted with DCM (3×10 mL). The combined organic layers were washed with brine (3×20 mL), dried (Na₂SO₄), and concentrated under reduced pressure. The residue was subjected to chromatography on silica gel using 0-100% CMA80 in DCM to furnish amide 5 (48 mg, 69%) as a white solid. ¹H NMR (300 MHz, CDCl₃) δ 7.51-7.34 (m, 3H), 7.18-7.03 (m, 2H), 6.07 (s, 1H), 4.69-4.56 (m, 1H), 3.63 (s, 3H), 3.29 (dd, J=11.1, 5.5 Hz, 2H), 2.91 (d, J=11.1 Hz, 2H), 2.44 (t, J=5.7 Hz, 2H), 2.33 (t, J=7.2 Hz, 2H), 2.24-2.12 (m, 4H), 1.98-1.85 (m, 2H), 1.84-1.76 (m, 2H), 1.74 (s, 3H), 1.52-1.33 (m, 2H); ¹³C NMR (75 MHz, CDCl₃) δ 173.6, 172.0, 170.2, 139.4, 130.1, 129.4, 128.4, 56.6, 52.9, 52.0, 51.5, 36.0, 35.3, 33.1, 30.2, 23.4, 20.8; MS (ESI) m/z: calculated for C₂₁H₃₁N₃O₄ 389.49, found 390.4 (M+H)⁺.

### Lithium 5-oxo-5-((2-(4-(N-phenylacetamido)piperidin-1-yl)ethyl)amino)pentanoate (6)

To a solution of the ester 5 (1.18 g, 3.03 mmol) in THF/MeOH/H₂O (50 mL, 1:1:0.5, v/v/v), LiOH.H₂O (0.16 g, 3.79 mmol) was added and the reaction, which resulted, was stirred at room temperature for 6 hours. The solvent was removed under nitrogen flow to provide lithium salt 6 (1.02 g, quant.) as a white solid. This material was used for the next transformation without any purification. ¹H NMR (300 MHz, CD₃OD) δ 7.57-7.42 (m, 3H), 7.24 (d, J=6.8 Hz, 2H), 4.62-4.49 (m, 1H), 3.26 (t, J=6.7 Hz, 2H), 2.98 (d, J=11.3 Hz, 2H), 2.43 (t, J=6.7 Hz, 2H), 2.24-2.08 (m, 6H), 1.88-1.78 (m, 4H), 1.76 (s, 3H), 1.43 (dt, J=12.0, 9.4 Hz, 2H); ¹³C NMR (75 MHz, CD₃OD) δ 181.8, 176.0, 172.6, 140.4, 131.3, 130.7, 130.0, 58.1, 54.1, 54.0, 38.4, 37.7, 37.0, 31.3, 24.1, 23.6; MS (ESI) m/z: calculated for C₂₀H₂₉N₃O₄ 375.47, found 376.4 (M+H)⁺.

### Methyl 4,7,10,13,17-pentaoxo-20-(4-(N-phenylacetamido)piperidin-1-yl)-3,6,9,12,18-pentaazaicosan-1-oate (7)

The Lithium salt 6 (0.62 g, 1.62 mmol) and BOP (1.07 g, 2.43 mmol) was dissolved in DMF (5 mL) and cooled to 0° C. A solution of TEA (0.34 mL, 2.43 mmol) and gly₄OMe (0.53 g, 2.03 mmol) in DMF (5 mL) was added dropwise to the above reaction at 0° C. The reaction was stirred at room temperature for 8 hours. The solvent was removed under nitrogen and the residue was subjected to chromatography on silica gel using 0-100% CMA80 in DCM to furnish amide 7 (413 mg, 41%) as a white solid. ¹H NMR (300 MHz, DMSO-d₆) δ 8.27 (t, J=5.8 Hz, 1H), 8.22-8.06 (m, 3H), 7.94 (t, J=5.3 Hz, 1H), 7.55-7.42 (m, 3H), 7.27 (d, J=6.8 Hz, 2H), 4.68 (t, J=11.9 Hz, 1H), 3.85 (d, J=5.9 Hz, 2H), 3.79-3.69 (m, 6H), 3.63 (s, 3H), 3.49-3.34 (m, 3H), 3.34-3.24 (m, 2H), 3.03-2.87 (m, 3H), 2.10 (dt, J=14.5, 7.4 Hz, 4H), 1.92 (d, J=12.2 Hz, 2H), 1.77-1.67 (m, 2H), 1.64 (s, 3H), 1.46 (dd, J=23.5, 12.0 Hz, 2H); ¹³C NMR (75 MHz, DMSO-d₆) δ 172.5, 172.3, 170.1, 169.5, 169.3, 169.1, 168.9, 138.8, 130.2, 129.5, 128.5, 55.2, 51.6, 51.5, 49.0, 42.0, 41.7, 40.5, 34.5, 34.3, 33.9, 27.4, 23.0, 21.0; MS (ESI) m/z: calculated for C₂₉H₄₃N₇O₈ 617.69, found 618.6 (M+H)⁺. HPLC (220 nm) t_{R}=9.05 min.

### Lithium 4,7,10,13,17-pentaoxo-20-(4-(N-phenylacetamido)piperidin-1-yl)-3,6,9,12,18-pentaazaicosan-1-oate (F₁₀)

To a solution of the ester 7 (144 mg, 0.23 mmol) in THF/MeOH/H₂O (5 mL, 1:1:0.5, v/v/v), LiOH.H₂O (14 mg, 0.35 mmol) was added and the reaction, which resulted, was stirred at room temperature. After complete consumption of starting material, as indicated by TLC, the reaction was evaporated to dryness under nitrogen flow to provide the lithium salt F₁₀ (145 mg, quant.) as a white solid. ¹H NMR (300 MHz, DMSO-d₆) δ 8.41-8.30 (m, 2H), 8.21 (t, J=5.7 Hz, 1H), 7.77 (t, J=5.5 Hz, 1H), 7.51-7.39 (m, 3H), 7.32 (t, J=4.5 Hz, 1H), 7.20 (d, J=6.6 Hz, 2H), 4.48-4.34 (m, 1H), 3.79-3.62 (m, 6H), 3.35 (s, 2H, merged with DMSO-H₂O), 3.07 (dd, J=12.3, 6.2 Hz, 2H), 2.83 (d, J=11.4 Hz, 2H), 2.25 (t, J=6.6 Hz, 2H), 2.10-1.90 (m, 6H), 1.72-1.63 (m, 4H), 1.61 (s, 3H), 1.27-1.10 (m, 2H); ¹³C NMR (75 MHz, DMSO-d₆) δ 172.3, 171.6, 170.4, 169.8, 169.1, 168.5, 167.6, 139.3, 130.2, 129.2, 128.2, 56.8, 52.6, 51.6, 43.9, 42.2, 42.2, 42.0, 36.1, 34.6, 34.4, 30.0, 23.1, 21.4; MS (ESI) m/z: calculated for C₂₈H₄₁N₇O₈ 603.67, found 604.6 (M+H)⁺. HPLC (220 nm) t_{R}=8.57 min.

### Synthesis of the F₁₁ Hapten.

To a mixture of N-benzylnorcarfentanil and HCOONH₄ under N₂ was added 18 mL of anhydrous MeOH. The resulting white suspension was degassed for 1 hour. Catalyst palladium on carbon (Pd/C) was added and the mixture was heated at reflux for 3 hours. LC-MS showed the reaction was complete. The reaction was removed from the heat and maintained overnight at ambient temperature. The crude reaction mixture was filtered through Celite to remove the catalyst. The Celite pad was washed with MeOH (100 mL) and the filtrate was concentrated. The residue was dissolved in CH₂Cl₂ (60 mL) and diluted with saturated aqueous NaHCO₃ (50 mL). The diluted residue was transferred to a separatory funnel and the layers were separated. The aqueous layer was further extracted with CH₂Cl₂ (2×25 mL). The combined organics were then dried over MgSO4, filtered, and concentrated to afford 385 mg (89%) of a viscous, pale yellow oil. This product was used in the next step without further purification.

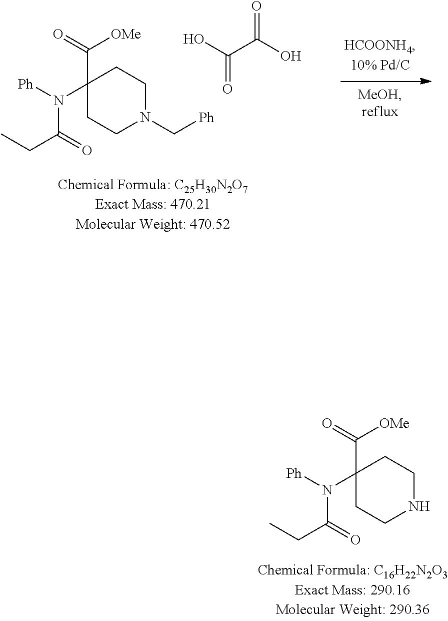

To a solution of norcarfentanil in 2 mL of anhydrous CH₃CN was added t-butyl acrylate via syringe at ambient temperature. The reaction was maintained for 20 hours. LC-MS analysis showed the reaction was complete. The crude reaction mixture was concentrated to afford a viscous, yellow oil. Purification by flash chromatography on SiO₂ (42 g, 80:20 EtOAc/hexanes) afforded 425 mg (77%) of the desired product as a viscous, pale yellow oil.

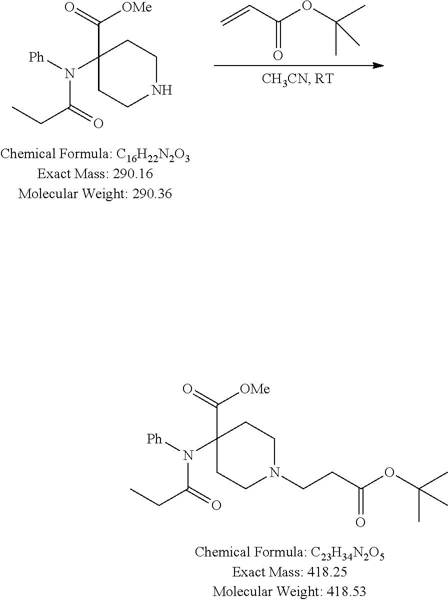

Ester (0.410 g, 0.980 mmol, 1.0 equiv) was treated with 4.5 mL of TFA (58.8 mmol, 60 equiv) at ambient temperature. Maintained for 90 minutes. LC-MS showed consumption of the starting material. Concentrated to furnish a viscous, yellow oil. Triturated with dry Et₂O to yield a white precipitate. Filtered and dried to obtain 398 mg (85%) of the desired product as a white solid.

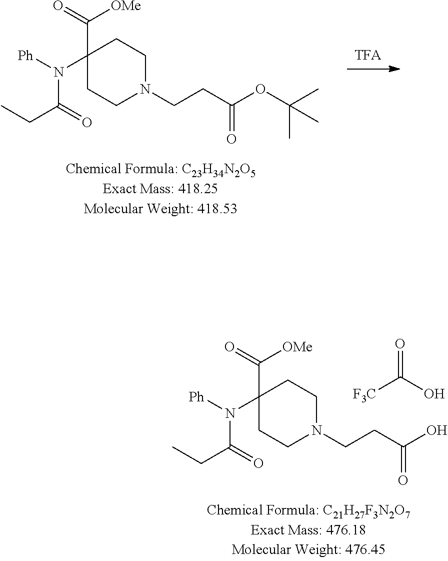

To a 0° C. suspension of acid (0.391 g, 0.821 mmol, 1.0 equiv) in 8 mL of anhydrous CH₂Cl₂ was added N-Boc ethylenediamine (0.195 mL, 1.23 mmol, 1.5 equiv) via pipette. The suspension became a solution. 1-Ethyl-3-(3-dimethylaminopropyl)carbodiimide (EDC) and dimethylaminopyridine (DMAP) were added and the reaction mixture was maintained at ambient temperature for 20 hours. The reaction was diluted with CH₂Cl₂ (80 mL) and washed with saturated aqueous NaHCO₃, H₂O, and brine. The organics were dried over anhydrous MgSO₄, filtered and concentrated. Purification by flash chromatography on SiO₂ (45 g, 6% MeOH/CH₂Cl₂) afforded 389 mg (94%) of the desired product as a white foam.

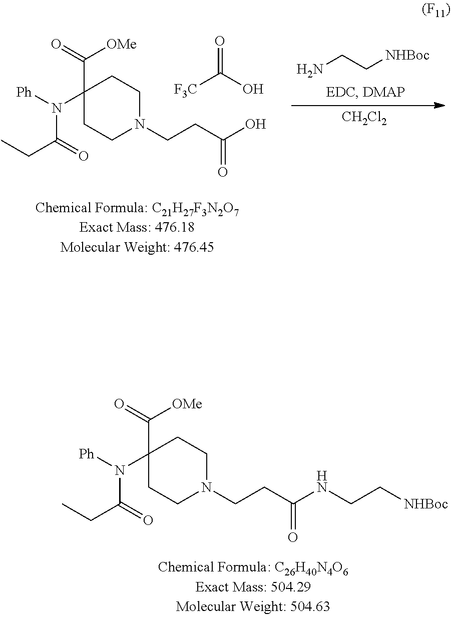

To a solution of N-Boc amine (0.380 g, 0.753 mmol, 1.0 equiv) in 1.0 mL of anhydrous CH2Cl2 at ambient temperature was added 85% H3PO4 dropwise via syringe. The reaction mixture was maintained at room temperature for 30 minutes with vigorous stirring. An oily whitish residue formed. LC-MS showed the reaction was nearly complete. After 1 hour, LC-MS showed the complete consumption of starting material. The reaction was diluted with H₂O (3 mL) after 80 min of total reaction time. Cooled to 0° C. Added 5 N NaOH (˜2 mL) to bring to pH ˜8-9. Extracted with CH₂Cl₂ (5×10 mL). Combined organics were dried over anhydrous MgSO₄, filtered, and concentrated. 189 mg (62%) of a white foam (F₁₁) was obtained.

### Synthesis of the F₁₂ Hapten

The F₁₂ hapten was synthesized as depicted in step (a) to step (e) of FIG. 1N. In FIG. 1N, the reagents and conditions used were as follows and as further described below: step a) 1-phenethyl-4-piperidone, AcOH, Na(OAc)₃BH, DCM:THF, 0° C. to room temperature, 14 hours; step b) propionic anhydride, DIPEA, DCM, 0° C. to room temperature, overnight; step c) HCl (4 M in dioxane), DCM, 0° C. to room temperature, 24 hours; step d) 4-nitrophenyl chloroformate, DIPEA, gly₄OMe, THF, 0° C. to room temperature, 2 hours; step e) LiOH.H₂O, THF/MeOH/H₂O, 24 hours.

### tert-Butyl (3-((1-phenethylpiperidin-4-yl)amino)phenyl)carbamate (1)

A solution of N-Boc-m-phenylenediamine (4.10 g, 19.68 mmol) in DCM:THF (160 mL, 1:1, v/v) was cooled to 0° C. and acetic acid (1.12 mL, 19.68 mmol) was added dropwise to the above solution. After that, 1-phenethyl-4-piperidone (4.0 g, 19.68 mmol) was added, followed by sodium triacetoxyborohydride (6.26 g, 29.52 mmol) in three portions at 0° C. The reaction was warmed and stirred at room temperature for 14 hours. Upon completion, the reaction was quenched with saturated aq. NaHCO₃ (50 mL). The layers were separated, and the aqueous layer was extracted with additional DCM (3×50 mL). The combined organic layers were washed with brine (2×50 mL), dried (Na₂SO₄), and concentrated under reduced pressure. The residue was subjected to chromatography on silica gel using 0-50% CMA80 in DCM to furnish compound 1 (6.0 g, 77%) as a brown solid. ¹H NMR (300 MHz, CDCl₃) δ 7.31-7.22 (m, 2H), 7.21-7.14 (m, 3H), 7.01 (t, J=8.0 Hz, 1H), 6.87 (s, 2H), 6.48 (dd, J=7.9, 1.3 Hz, 1H), 6.24 (dd, J=8.0, 1.7 Hz, 1H), 3.56 (d, J=8.2 Hz, 1H), 3.35-3.18 (m, 1H), 2.98-2.83 (m, 2H), 2.83-2.70 (m, 2H), 2.63-2.50 (m, 2H), 2.14 (t, J=10.5 Hz, 2H), 2.07-1.94 (m, 2H), 1.57-1.37 (m, 11H); ¹³C NMR (75 MHz, CDCl₃) δ 152.9, 148.0, 140.5, 139.7, 129.7, 128.8, 128.5, 126.1, 107.9, 107.1, 103.2, 80.2, 60.7, 52.4, 49.8, 33.9, 32.5, 28.5; MS (ESI) m/z: calculated for C₂₄H₃₃N₃O₂ 395.54, found 396.6 [M+H]⁺.

### tert-Butyl (3-(N-(1-phenethylpiperidin-4-yl)propionamido)phenyl)carbamate (2)

To a solution of amine 1 (6.0 g, 15.17 mmol) in dry DCM (80 mL), DIPEA (5.19 mL, 30.34 mmol) and propionic anhydride (7.74 mL, 60.68 mmol) was added at 0° C. The reaction which resulted, was stirred overnight at room temperature. At this point, the reaction was cooled to 0° C. and quenched with saturated aq. NaHCO₃ (30 mL). The layers were separated, and the aqueous layer was extracted with additional DCM (3×60 mL). The combined organic layers were washed with brine (3×50 mL), dried (Na₂SO₄), and concentrated under reduced pressure to give a yellowish residue. The residue was subjected to chromatography on silica gel using 0-50% CMA80 in DCM to furnish amide 2 (6.15 g, 89%) as a yellow solid. ¹H NMR (300 MHz, CDCl₃) δ 7.36-7.12 (m, 8H), 6.82-6.69 (m, 2H), 4.72-4.60 (m, 1H), 3.11 (d, J=10.6 Hz, 2H), 2.84-2.73 (m, 2H), 2.67-2.54 (m, 2H), 2.31-2.17 (m, 2H), 1.99 (q, J=7.4 Hz, 2H), 1.92-1.70 (m, 2H), 1.59-1.42 (m, 11H), 1.02 (t, J=7.4 Hz, 3H); MS (ESI) m/z: calculated for C₂₇H₃₇N₃O₃ 451.60, found 452.6 [M+H]⁺.

### N-(3-aminophenyl)-N-(1-phenethylpiperidin-4-yl)propionamide (3)

To an ice-cold solution of carbamate 2 (6.15 g, 13.62 mmol) in dry DCM (50 mL), HCl (17.02 mL, 4 M solution in dioxane) was added. The resulting reaction was stirred at room temperature for 24 hours. The solvent was removed under reduced pressure and the residue was subjected to chromatography on silica gel using 0-100% CMA80 in DCM to furnish amine 3 (3.88 g, 81%) as a white solid. ¹H NMR (300 MHz, CDCl₃) δ 7.35-7.17 (m, 5H), 7.11 (t, J=7.9 Hz, 1H), 6.71 (dd, J=8.0, 1.6 Hz, 1H), 6.50-6.45 (m, 1H), 6.37 (d, J=7.7 Hz, 1H), 4.80-4.60 (m, 1H), 3.50 (d, J=11.4 Hz, 2H), 3.15 (s, 4H), 2.97 (t, J=11.4 Hz, 2H), 2.21-1.76 (m, 6H), 0.99 (t, J=7.4 Hz, 3H); ¹³C NMR (75 MHz, CDCl₃) δ 174.0, 148.5, 138.8, 136.4, 130.1, 128.8, 128.7, 127.1, 118.8, 115.7, 115.3, 58.0, 52.1, 50.1, 30.4, 28.0, 27.5, 9.6; MS (ESI) m/z: calculated for C₂₂H₂₉N₃O 351.49, found 352.40 [M+H]⁺.

### Methyl 1,4,7,10-tetraoxo-1-((3-(N-(1-phenethylpiperidin-4-yl)propionamido)phenyl)amino)-2,5,8,11-tetraazatridecan-13-oate (4)

To a suspension of amine 3 (0.50 g, 1.42 mmol) in THF (20 mL), DIPEA (0.62 mL, 3.56 mmol) was added. The reaction was then cooled to 0° C., and a solution of 4-nitrophenyl chloroformate (0.29 g, 1.42 mmol) in THF (6 mL) was added dropwise. After that, the reaction was stirred at 0° C. for 30 minutes. At this point, a solution of gly₄OMe (0.44 g, 1.71 mmol) and DIPEA (0.62 mL, 3.56 mmol) in THF (20 mL) was added to the above reaction. The reaction was then stirred for 30 minutes at 0° C. and an additional 1.5 hours at room temperature. The reaction was then quenched with MeOH (5 mL) and concentrated to dryness under nitrogen flow. The residue was subjected to chromatography on silica gel using 0-100% CMA80 in DCM to furnish urea 4 (0.34 g, 38%) as a white solid. ¹H NMR (300 MHz, DMSO-d₆) δ 9.09 (s, 1H), 8.39-8.14 (m, 3H), 7.44-7.08 (m, 8H), 6.81-6.67 (m, 1H), 6.48 (t, J=5.3 Hz, 1H), 4.54-4.38 (m, 1H), 3.92-3.71 (m, 8H), 3.62 (s, 3H), 3.14-2.93 (m, 2H), 2.79-2.53 (m, 4H), 2.36-2.01 (m, 2H), 1.87 (dd, J=14.6, 7.2 Hz, 2H), 1.70 (d, J=10.1 Hz, 2H), 1.45-1.16 (m, 2H), 0.89 (t, J=7.4 Hz, 3H); ¹³C NMR (75 MHz, DMSO-d₆) δ 171.8, 170.1, 170.0, 169.3, 169.1, 155.1, 141.2, 139.8, 139.0, 129.3, 128.5, 128.2, 125.9, 122.8, 119.1, 117.2, 58.9, 52.3, 51.6, 51.3, 42.6, 42.0, 41.67, 40.5, 32.4, 29.6, 27.6, 9.5; MS (ESI) m/z: calculated for C₃₂H₄₃N₇O₇ 637.73, found 638.8 [M+H]⁺. HPLC (280 nm) t_{R}=10.61 min.

### Lithium 1,4,7,10-tetraoxo-1-((3-(N-(1-phenethylpiperidin-4-yl)propionamido)phenyl)amino)-2,5,8,11-tetraazatridecan-13-oate (F₁₂)

To a solution of the ester 4 (54 mg, 0.08 mmol) in THF/MeOH/H₂O (5 mL, 1:1:0.5, v/v/v), LiOH.H₂O (5.0 mg, 0.11 mmol) was added and the reaction, which resulted, was stirred at room temperature for 24 hours. After complete consumption of starting material, as indicated by TLC, the reaction was concentrated to dryness under nitrogen flow to provide lithium salt F₁₂ (54 mg, quant.) as a white solid. ¹H NMR (300 MHz, CD₃OD) δ 7.51-7.28 (m, 3H), 7.28-7.09 (m, 5H), 6.79 (d, J=7.5 Hz, 1H), 4.53 (t, J=11.9 Hz, 1H), 4.00-3.88 (m, 5H), 3.84-3.66 (m, 3H), 3.13-2.92 (m, 2H), 2.78-2.64 (m, 2H), 2.56-2.41 (m, 2H), 2.16 (t, J=11.3 Hz, 2H), 2.02 (dd, J=13.7, 7.3 Hz, 2H), 1.82 (d, J=9.6 Hz, 2H), 1.60-1.34 (m, 2H), 0.99 (t, J=7.5 Hz, 3H); ¹³C NMR (75 MHz, CD₃OD) δ 176.3, 175.9, 174.0, 172.4, 171.3, 158.2, 142.5, 141.3, 140.3, 130.7, 129.7, 129.5, 127.1, 124.7, 121.5, 119.9, 61.4, 54.0, 44.4, 44.3, 44.1, 43.7, 34.2, 31.2, 29.4, 10.2; MS (ESI) m/z: calculated for C₃₁H₄₁N₇O₇ 623.70, found 624.8 [M+H]⁺. HPLC (280 nm) t_{R}=10.15 min.

### Synthesis of the F₁₃ Hapten

The F₁₃ hapten was synthesized as depicted in step (a) to step (i) of FIG. 1O. In FIG. 1O, the reagents and conditions used were as follows and as further described below: step a) N-Boc-4-piperidone, NaOH, CHCl₃, THF, 0° C. to room temperature, overnight; step b) propionic anhydride, TEA, EtOAc, MeOH, reflux, 5 hours; step c) HCl (4 M in dioxane), DCM, 0° C. to room temperature, 20 hours; step d) N-Boc-2-aminoacetaldehyde, Na(OAc)₃BH, 1,2-DCE, 0° C. to room temperature, 20 hours; step e) TFA, DCM, 0° C. to room temperature, overnight; step f) 5-(benzyloxy)-5-oxopentanoic acid, oxalyl chloride, DCM, DMF (cat.), 0° C. to room temperature, 3 hours; step g) benzyl 5-chloro-5-oxopentanoate 6, TEA, DCM, 0° C. to room temperature, overnight; step h) Pd (10% on C), H₂ (40 psi), MeOH, room temperature, overnight; step i) gly₄Cbz, BOP, TEA, DMF, 0° C. to room temperature, 16 hours; step j) Pd (10% on C), H₂ (1 atm), MeOH, room temperature, overnight.

### 1-(tert-Butoxycarbonyl)-4-(phenylamino)piperidine-4-carboxylic acid (1)

To a solution of aniline (2.0 g, 21.48 mmol) in THF (60 ml), NaOH (fine powder, 4.29 g, 107.38 mmol) and N-Boc-4-piperidone (8.56 g, 42.95 mmol) were added at 0° C. CHCl₃ (8.6 mL, 107.38 mmol) was added dropwise via a syringe and the reaction mixture was stirred at 0° C. for 1 hour and at room temperature overnight. The reaction was filtered, and the filter cake was dissolved in water (50 mL). The aqueous solution was extracted with Et₂O (3×50 mL) to retain non-polar impurities. The aqueous layer was cooled to 0° C. and acidified to ˜pH 3 with HCl (2 N) and extracted with EtOAc (2×50 mL). The combined organic layers were washed with brine (3×30 mL), dried (Na₂SO₄), and concentrated in vacuo to furnish the carboxylic acid 1 (5.36 g, 78%) as a yellow solid. This material was used for the next transformation without any purification. ¹H NMR (300 MHz, CDCl₃) δ 7.15 (t, J=7.8 Hz, 2H), 6.79 (t, J=7.3 Hz, 1H), 6.64 (d, J=7.9 Hz, 2H), 3.78-3.62 (m, 2H), 3.37-3.19 (m, 2H), 2.16-2.09 (m, 1H), 2.03 (s, 1H), 2.02-1.91 (m, 2H), 1.44 (s, 9H); ¹³C NMR (75 MHz, CDCl₃) δ 179.0, 155.0, 144.3, 129.2, 119.6, 116.3, 80.2, 58.6, 39.2, 32.4, 28.4; MS (ESI) m/z: calculated for C₁₇H₂₄N₂O₄ 320.38, found 321.2 [M+H]⁺.

### 1-tert-Butyl 4-methyl 4-(N-phenylpropionamido)piperidine-1,4-dicarboxylate (2)

To a stirred suspension of acid 1 (3.83 g, 11.94 mmol) and propionic anhydride (10.67 mL, 83.59 mmol) in EtOAc (50 mL) at reflux, TEA (5.0 mL, 35.83 mmol) was added slowly and the reaction, which resulted was heated at reflux for 2 hours. The reaction mixture was cooled to 70° C. and MeOH (6 mL, excess) was added and the heating was continued for additional 3 hours. After that, the reaction was cooled to room temperature and basified with aq. NaOH (3 M). The organic layer was separated, and the aqueous layer was extracted with EtOAc (2×50 mL). The combined organic layers were washed with brine (3×30 mL), dried (Na₂SO₄), and concentrated under reduced pressure. The residue was subjected to chromatography on silica gel using 0-50% CMA80 in EtOAc to furnish amido ester 2 (3.60 g, 77%) as a pale yellow solid. ¹H NMR (300 MHz, CDCl₃) δ 7.50-7.39 (m, 3H), 7.34-7.24 (m, 2H), 3.87-3.69 (m, 5H), 3.17 (br s, 2H), 2.31-2.13 (m, 2H), 1.89 (q, J=7.4 Hz, 2H), 1.54-1.38 (m, 11H), 0.96 (t, J=7.4 Hz, 3H); ¹³C NMR (75 MHz, CDCl₃) δ 174.2, 173.6, 154.7, 139.2, 130.5, 129.4, 128.9, 79.6, 62.9, 52.2, 40.4, 33.1, 28.9, 28.3, 9.1; MS (ESI) m/z: calculated for C₂₁H₃₀N₂O₅Na 413.46, found 413.4 [M+Na]⁺.

### Methyl 4-(N-phenylpropionamido) piperidine-4-carboxylate (3)

To an ice-cold solution of carbamate 2 (3.60 g, 9.22 mmol) in dry DCM (30 mL), HCl (11.5 mL, 4 M solution in dioxane) was added. The reaction, which resulted, was stirred at room temperature for 20 hours. The solvent was removed under reduced pressure and the residue was subjected to chromatography on silica gel using 0-100% CMA80 in DCM to furnish amine 3 (2.32 g, 87%) as a white solid. ¹H NMR (300 MHz, CDCl₃) δ 7.50-7.38 (m, 3H), 7.37-7.30 (m, 2H), 3.81 (s, 3H), 3.50-3.43 (m, 1H), 3.26-3.07 (m, 4H), 2.46-2.30 (m, 2H), 1.99-1.80 (m, 4H), 0.95 (t, J=7.4 Hz, 3H); ¹³C NMR (75 MHz, CDCl₃) δ 174.4, 173.1, 138.6, 130.5, 129.7, 129.2, 61.3, 52.6, 41.4, 31.1, 29.0, 9.1; MS (ESI) m/z: calculated for C₁₆H₂₂N₂O₃ 290.36, found 291.4 [M+H]⁺.

### Methyl 1-(2-((tert-butoxycarbonyl)amino)ethyl)-4-(N-phenylpropionamido)piperidine-4-carboxylate (4)

To a solution of amine 3 (1.76 g, 6.06 mmol) in 1,2-DCE (30 mL), N-Boc-2-aminoacetaldehyde (1.16 g, 7.27 mmol, dissolved in 5 mL DCE) was added dropwise at 0° C. Sodium triacetoxyborohydride (1.93 g, 9.09 mmol) was added to the above reaction in two portions at 0° C. and the reaction, which resulted, was stirred at room temperature for 20 hours. Upon completion, the reaction was quenched with saturated aq. NaHCO₃ (20 mL). The organic layer was separated, and the aqueous layer was extracted with DCM (3×30 mL). The combined organic layers were washed with brine (3×30 mL), and dried (Na₂SO₄). The solvent was removed under reduced pressure and the residue was subjected to chromatography on silica gel using 0-50% CMA80 in DCM to furnish compound 4 (1.93 g, 73%) as a white solid. ¹H NMR (300 MHz, CDCl₃) δ 7.50-7.38 (m, 3H), 7.37-7.28 (m, 2H), 5.14-5.03 (m, 1H), 3.79 (s, 3H), 3.22-3.08 (m, 2H), 2.71-2.54 (m, 2H), 2.47-2.35 (m, 4H), 2.27 (d, J=13.2 Hz, 2H), 1.88 (q, J=7.4 Hz, 2H), 1.70-1.52 (m, 2H), 1.42 (s, 9H), 0.95 (t, J=7.4 Hz, 3H); ¹³C NMR (75 MHz, CDCl₃) δ 174.0, 173.8, 155.8, 139.3, 130.5, 129.3, 128.7, 78.9, 62.7, 57.0, 52.0, 49.6, 37.2, 33.3, 28.9, 28.3, 9.1; MS (ESI) m/z: calculated for C₂₃H₃₅N₃O₅ 433.54, found 434.4 [M+H]⁺.

### Methyl 1-(2-aminoethyl)-4-(N-phenylpropionamido)piperidine-4-carboxylate (5)

To a solution of carbamate 4 (1.93 g, 4.45 mmol) in DCM (20 mL), TFA (1.36 mL, 17.81 mmol) was added at 0° C. and the reaction, which resulted, was stirred at room temperature overnight. The solvent was removed with nitrogen flow. The residue was subjected to chromatography on silica gel using 0-100% CMA80 in DCM to furnish amine 5 (1.28 g, 87%) as a white solid. ¹H NMR (300 MHz, CD₃OD) δ 7.57-7.45 (m, 3H), 7.44-7.33 (m, 2H), 3.80 (s, 3H), 3.26-3.10 (m, 4H), 3.10-2.90 (m, 4H), 2.40 (d, J=14.0 Hz, 2H), 1.99-1.80 (m, 4H), 0.93 (t, J=7.4 Hz, 3H); ¹³C NMR (75 MHz, CD₃OD) δ 176.7, 174.6, 139.8, 131.5, 130.9, 130.5, 62.7, 54.9, 53.1, 51.1, 50.0, 48.3, 36.3, 32.8, 29.9, 9.7; MS (ESI) m/z: calculated for C₁₈H₂₇N₃O₃ 333.43, found 334.4 [M+H]⁺.

### Methyl 1-(2-(5-(benzyloxy)-5-oxopentanamido)ethyl)-4-(N-phenylpropionamido)piperidine-4-carboxylate (7)

To a solution of 5-(benzyloxy)-5-oxopentanoic acid (1.27 g, 5.71 mmol) in DCM (20 mL) at 0° C., oxalyl chloride (2.4 mL, 5 eq) was added, followed by DMF (cat.). The resulting reaction was stirred at room temperature for 3 hours. The solvent was removed under reduced pressure to furnish benzyl 5-chloro-5-oxopentanoate 6 (1.37 g, quant.) as a yellowish residue, which was used directly for the next transformation. To a solution of the acid chloride 6 (1.37 g, 5.71 mmol) in dry DCM, TEA (3.2 mL, 22.85 mmol) was added at 0° C., followed by amine 5 (1.52 g, 4.57 mmol). The reaction was stirred overnight at room temperature. The solvent was removed under reduced pressure and the residue was subjected to chromatography on silica gel using 0-100% CMA80 in DCM to furnish amide 7 (1.33 g, 54%) as a white solid. ¹H NMR (300 MHz, CDCl₃) δ 7.49-7.28 (m, 10H), 6.16-6.06 (m, 1H), 5.10 (s, 2H), 3.78 (s, 3H), 3.25 (dd, J=11.3, 5.7 Hz, 2H), 2.66-2.52 (m, 2H), 2.48-2.35 (m, 6H), 2.33-2.13 (m, 4H), 2.01-1.82 (m, 4H), 1.69-1.53 (m, 2H), 0.95 (t, J=7.4 Hz, 3H); ¹³C NMR (75 MHz, CDCl₃) δ 174.1, 173.8, 173.0, 172.0, 139.3, 135.9, 130.6, 129.3, 128.7, 128.5, 128.2, 128.1, 66.1, 62.7, 56.5, 52.1, 49.6, 36.1, 35.2, 33.3, 33.3, 29.0, 20.8, 9.1; MS (ESI) m/z: calculated for C₃₀H₃₉N₃O₆ 537.65, found 538.6 [M+H]⁺.

### 5-((2-(4-(methoxycarbonyl)-4-(N-phenylpropionamido)piperidin-1-yl)ethyl)amino)-5-oxopentanoic acid (8)

To a solution of benzyl ester 7 (0.65 g, 1.21 mmol) in MeOH (20 mL), Pd (0.26 g, 0.24 mmol, 10% on C) was added and the mixture was hydrogenated overnight (40 psi) at room temperature. The reaction mixture was filtered through Celite, and the filtrate was concentrated to dryness to provide acid 8 (0.47 g, 86%) as a white solid. ¹H NMR (300 MHz, CD₃OD) δ 7.45-7.34 (m, 3H), 7.31-7.25 (m, 2H), 3.68 (s, 3H), 3.32-3.19 (m, 2H, merged with solvent peak), 3.07-2.89 (m, 2H), 2.78-2.57 (m, 4H), 2.32-2.18 (m, 2H), 2.08 (q, J=7.1 Hz, 4H), 1.88-1.68 (m, 6H), 0.83 (t, J=7.4 Hz, 3H); ¹³C NMR (75 MHz, CD₃OD) δ 180.4, 176.6, 176.1, 174.7, 140.1, 131.7, 130.8, 130.4, 63.2, 57.7, 52.9, 50.8, 37.2, 36.7, 36.4, 32.9, 29.9, 23.1, 9.6; MS (ESI) m/z: calculated for C₂₃H₃₃N₃O₆ 447.52, found 448.4 [M+H]⁺.

### Methyl 1-(3,6,9,12,15,19-hexaoxo-1-phenyl-2-oxa-5,8,11,14,20-pentaazadocosan-22-yl)-4-(N-phenylpropionamido)piperidine-4-carboxylate (9)

To a solution of acid 8 (0.10 g, 0.22 mmol) in DMF (5 mL), BOP (0.15 g, 0.34 mmol) was added at 0° C. After that, a solution of TEA (0.1 mL, 0.67 mmol) and gly₄Cbz tosylate salt (0.12 g, 0.25 mmol) in DMF (5) was added dropwise to the above reaction at 0° C. The reaction was stirred at room temperature for 16 hours. The solvent was removed under nitrogen flow and the residue was subjected to chromatography on silica gel using 0-100% CMA80 in DCM to furnish amide 9 (62.8 mg, 37%) as a white solid. ¹H NMR (300 MHz, DMSO-d₆) δ 8.30 (t, J=5.9 Hz, 1H), 8.22-8.07 (m, 3H), 7.70 (t, J=5.4 Hz, 1H), 7.54-7.43 (m, 3H), 7.41-7.29 (m, 7H), 5.13 (s, 2H), 3.91 (d, J=5.9 Hz, 2H), 3.79-3.68 (m, 6H), 3.65 (s, 3H), 3.07 (dd, J=12.2, 6.3 Hz, 2H), 2.60-2.44 (m, 2H, merged with solvent peak), 2.30-2.17 (m, 4H), 2.14-1.95 (m, 6H), 1.84-1.60 (m, 4H), 1.59-1.42 (m, 2H), 0.82 (t, J=7.4 Hz, 3H); ¹³C NMR (75 MHz, DMSO-d₆) δ 173.0, 172.7, 172.3, 171.5, 169.6, 169.6, 169.3, 169.0, 139.1, 135.9, 130.5, 129.3, 128.6, 128.4, 128.0, 127.8, 65.8, 61.9, 56.8, 54.8, 51.7, 49.2, 42.0, 41.7, 40.6, 36.0, 34.6, 34.4, 32.8, 28.3, 21.4, 9.1; MS (ESI) m/z: calculated for C₃₈H₅₁N₇O₁₀ 765.85, found 766.8 [M+H]⁺. HPLC (220 nm) t_{R}=11.62 min.

### 20-(4-(methoxycarbonyl)-4-(N-phenylpropionamido)piperidin-1-yl)-4,7,10,13,17-pentaoxo-3,6,9,12,18-pentaazaicosan-1-oic acid (F₁₃)

To a solution of ester 9 (34 mg, 0.04 mmol) in MeOH (10 mL), Pd (9.0 mg, 0.01 mmol, 10% on C) was added and the mixture was hydrogenated (1 atm) overnight at room temperature. The reaction mixture was filtered through Celite, and the filtrate was concentrated to dryness under nitrogen flow to provide acid F₁₃ (28 mg, 93%) as a white solid. ¹H NMR (300 MHz, DMSO-d₆) δ 8.23-8.09 (m, 3H), 7.97 (t, J=5.4 Hz, 1H), 7.76 (t, J=5.3 Hz, 1H), 7.54-7.41 (m, 3H), 7.38-7.30 (m, 2H), 3.77-3.62 (m, 11H), 3.13-3.03 (m, 2H), 2.63-2.53 (m, 2H), 2.34-2.21 (m, 4H), 2.13-1.96 (m, 6H), 1.84-1.60 (m, 4H), 1.59-1.42 (m, 2H), 0.82 (t, J=7.4 Hz, 3H); ¹³C NMR (75 MHz, DMSO-d₆) δ 173.0, 172.8, 172.3, 171.6, 171.0, 169.6, 169.0, 168.7, 139.0, 130.4, 129.3, 128.7, 61.8, 56.7, 51.7, 49.2, 42.1, 42.0, 41.8, 41.1, 35.9, 34.6, 34.4, 32.6, 28.3, 21.3, 9.1; MS (ESI) m/z: calculated for C₃₁H₄₅N₇O₁₀ 675.7, found 676.60 [M+H]⁺. HPLC (220 nm) t_{R}=9.62 min.

### Example 5

### Materials and Methods

Hapten Synthesis. Haptens F₁ to F₇ were synthesized as described in Example 1. Hapten F₈ was synthesized as described in Example 2. Haptens F₉ₐ and F_{9b} was synthesized as described in Example 3B. Haptens F₁₀ to F₁₃ were synthesized as described in Example 4.

Conjugation of the F₁ and F₈₋₁₄ haptens to carrier proteins via either carbodiimide (EDAC) or NHS coupling chemistry. Conjugation and characterization of conjugates were described in Example 1 or as in Robinson et al. (Robinson et al. J Med Chem 63, 14647-14667 (2020)). Conjugates were characterized for molecular weight, size and aggregation status by MALDI-TOF, dynamic light scattering, and visual appearance.

Animals. All animal studies were approved by the University of Minnesota Institute Animal Care and Use Committee and conducted in AALAC-certified institutional facilities. Mice and rats were housed in standard 12/12 hours light/dark cycle and fed ad libitum. Mice were 6-7 weeks and rats were 2 months old on arrival. Immunization protocols were initiated after 1 week of habituation.

Active immunization. Mice and rats were immunized i.m. with either unconjugated carrier protein or conjugate vaccine containing the target hapten from the novel F₈₋₁₃ series. As detailed in each individual study, 3 independent experiments were performed to test the following lead haptens: 1) F₁, F₈, F₉ₐ, F_{9b}, and F₁₀; 2) F₁, F₆ and F₁₂; and 3) F₁, F₁₁, and F₁₃. In all studies, F₁-CRM was used as positive control. Conjugates were adsorbed to aluminum adjuvant (ALHYDROGEL) prior to injection. Mice were immunized on days 0, 14, and 28, whereas rats were immunized on days 0, 21, 42 and 63.

Antibody Analysis. Serum antibody analysis was performed via indirect ELISA after blood collection using tail vein sampling in rats. 96-well plates were coated with 5 ng/well of BSA conjugated to the corresponding F₁₋₁₃ hapten or unconjugated BSA as a control diluted in 50 mM Na₂CO₃, pH 9.6 (Sigma, St. Louis, Mo.) and blocked with 1% porcine gelatin. Plates were incubated with serum samples then washed and incubated with an HRP-conjugated goat anti-rat IgG (Jackson ImmunoResearch Laboratories, West Grove, Pa.) to assess hapten-specific serum IgG antibody levels. For determination of affinity by competitive binding ELISA, 96-well plates were coated and blocked as described above, and fentanyl or fentanyl analogs were added to the wells with concentrations ranging from 1×10⁻⁴ M to 1×10⁻¹⁰ M. Immune sera were diluted to sub-saturating concentrations, incubated with competitor on the plate and washed and incubated with HRP-conjugated antibody as above.

Vaccine efficacy against opioid-induced antinociception via hot plate test. To determine the efficacy of each candidate vaccine against opioid-induced antinociception, a hot plate test was performed as previously described (Robinson et al. J Med Chem 63, 14647-14667 (2020)). After completion of the immunization protocol, vaccinated mice and rats were challenged with a series of single subcutaneous (s.c.) doses of the target opioid. The drugs were given in a randomized fashion to mitigate tolerance effects. Prior to testing, mice and rats were allowed to acclimate to the testing environment for 1 hour prior to measuring baseline. Rodents were placed on a hot plate (Columbus Instruments, Columbus, Ohio) set to 54° C. and removed after displaying a lift or flick of the hindpaw. Active drug challenges were initiated after collection of individual baselines. Doses of drugs were as follows: fentanyl 0.1 mg/kg, alfentanil 0.25 and 0.5 mg/kg, acetylfentanyl 0.5 and 1 mg/kg, sufentanil 0.008 mg/kg, carfentanil 0.02 mg/kg. The latency to respond on the hotplate was measured at 15, 30, 45, and 60 minutes post-drug administration. Data are displayed as maximal possible effect (MPE) calculated as: (postdrug latency−baseline latency)/(maximal cutoff−baseline latency)×100.

Vaccine efficacy against opioid-induced respiratory depression and bradycardia. To assess the efficacy of candidate vaccines against opioid-induced respiratory depression and bradycardia, oxygen saturation and heart rate were measured by pulse oximetry before and after drug administration as previously described (Robinson et al. J Med Chem 63, 14647-14667 (2020)). Oximetry was measured using a MouseOx Plus pulse oximeter (Starr Life Sciences, Oakmont, Pa.). Rats were allowed to acclimate to the testing environment for 1 hour prior to measuring baseline. After baseline, rats were given a single bolus dose of drug s.c. in a randomized fashion as outlined for the hotplate test. Oximetry measurements were taken at 15, 30, 45, and 60 minutes post-drug administration.

Computational Methods. GLIDE docking grids were prepared based on the initial N-terminal truncated crystal structure coordinates with PropPKA protonation assignments. Two full length structures were prepared using MODELLER (Webb et al. Methods Mol Biol 1654, 39-54 (2017)) simulated annealing with topological constraints, nudged elastic band pulls of the complete sequence N- and C-terminal ends employing AMBER 18 parameterization (Case et al., AMBER 2018 (2018)) to generate more compact termini conformations followed by equilibration in a DOPC/K+/Cl−/water environment. A LIPID 14 membrane-component AMBER parameterization was used (Dickson et al. J Chem Theory Comput 10, 865-879 (2014)). Protein preparation including protonation assignment was performed on these initial structures with Small Molecule Discovery Suite software (Schrödinger, INC, New York N.Y.) prior to preparation of Glide (Friesner et al. J Med Chem 49, 6177-6196 (2006)) docking grids centered on residues near the extracellular region and including D147, Y148, and H54-proximate to the orthosteric binding site. H54 was included in the docking region to further examine the N-terminal-loop insertion into the orthosteric binding site of the 5C1M structure in a manner ‘analogous’ to what was observed in the initial CB1 antagonist crystal structures. The role of such insertions had not previously been explored in stabilization of the bound ligand binding conformation. For this reason, BU72 (4V0 in the 5C1M structure file) was docked into the truncated original coordinates and into two membrane equilibrated full length MOR structures, one with an elastic band pull of the N-terminus removing that N-terminal loop insertion and a second retaining the N-terminal loop insertion.

Statistical analysis. Serum antibody titers, drug serum or brain concentrations, latency to respond in the hotplate nociception test, oxygen saturation (SaO₂), and heart rate (beats per minute, BPM) on single time points were compared using a one-way ANOVA or two-way ANOVA overtime, and paired with appropriate post hoc tests. Analyses were performed using Prism (version 9.0; GraphPad, San Diego, Calif.).

### Results

Characterization of Biophysical Properties of Conjugate Vaccines. Biophysical properties of conjugate vaccines (including molecular weight (MW), measured by MALDI-TOF; estimated haptenation ratio; and whether the conjugate vaccine precipitates or aggregates) for haptens F₈ to F₁₃ were characterized. Results are shown in Table 1B.

Testing of Conjugate Vaccines in Pre-Clinical Models. Mice and rats were immunized, as described above.

FIG. 12 shows vaccine efficacy of haptens F₄ to F₆ against fentanyl in rats.

FIG. 13 shows vaccine efficacy of haptens F₁, F₈, F₉, and F₁₀ against fentanyl and sufentanil in rats. Statistical significance is shown in Table 5. FIG. 14 shows vaccine efficacy of haptens F₁, F₈, F₉, and F₁₀ against alfentanil in rats. Statistical significance is shown in Table 6. FIG. 15 shows vaccine efficacy of haptens F₁, F₈, F₉, and F₁₀ against acetylfentanyl in rats. Statistical significance is shown in Table 7.

FIG. 16 shows vaccine efficacy of haptens F₁, F₁₁, and F₁₃ against carfentanil in rats. Statistical significance is shown in Table 8. FIG. 17 shows vaccine efficacy of haptens F₁, F₁₁, and F₁₃ against fentanyl in rats. Statistical significance is shown in Table 9. FIG. 18 shows vaccine efficacy of haptens F₁, F₁₁, and F₁₃ against a combination of carfentanil and fentanyl in rats. Statistical significance is shown in Table 10.

FIG. 19 shows vaccine efficacy of haptens F₁, F₁₁, and F₁₃ against increasing doses of carfentanil in rats. Statistical significance is shown in Table 11.

FIG. 20 shows vaccine efficacy of haptens F₁, F₆, and F₁₂ against fentanyl in mice.

Activity of the fentanyl-based haptens at the Mu Opioid Receptor (MOR). As described in Example 1, the F₁ hapten has no functional agonist activity at the MOR, most likely due to the extended peptidic linker and lack of an N-phenylethyl substituent.

In contrast, the F₄ hapten—which includes the N-phenylethyl substituent (FIG. 9C)—exhibits functional agonist activity at the MOR Activity of the fentanyl-based hapten F₁ at the MOR. Results are shown in FIG. 9D and Table 4B.

In addition, F₁ was characterized for its potential to interact and activate the MOR in silico. To this end, the F₁ hapten was docked into the Kolbilka crystal structure (PDB: 5C1M) (Huang et al. J Chem Inf Model 52, 1356-1366 (2012)) for the agonist μ opiate receptor complexed with BU72 stabilized by a G-protein camelid antibody fragment Nb39 (PDB: 5C1M) which increases the agonist affinity of BU72 from 470 pM to 16 pM (Huang et al. J Chem Inf Model 52, 1356-1366 (2012)). The F₁ hapten showed the lowest Gscore pose. F₁ loosely fits the binding site with its core buried in the same region as prototypical MOR agonists fentanyl and BU72 and with its [Gly]₄ linker extending toward the extracellular loops. It is noteworthy that most F₁ poses did not possess the strong hydrogen bonding and π-cation interactions with D147/Y148 that BU72 and fentanyl induce. In 1-2 cases of less favorable Gscore/Emodel F₁ docking poses, F₁ picked up a hydrogen bond interaction with D147 but only at the expense of considerable ligand strain energy as indicated by positive Emodel Glide score values. The Gscore/Emodel scores paralleled the EC₅₀ values of the ligands. Although docking scores are not robust predictors of activation, they often parallel Molecular Mechanics Generalized Born Surface Area (MMGBSA) relative binding free energy scores. The qualitative conclusion that can be drawn from these results is that F₁ has some of the shape prerequisites to recognize the orthosteric binding site but does not possess key interactions that would induce activation changes in the receptor ensemble. F₁, fentanyl, and BU72 demonstrated a lack of functional activation for F₁ at the MOR, indicating a favorable profile for progression.

Affinity of antibodies for fentanyl and its analogs. Sera from mice and rats immunized with conjugates containing fentanyl-based haptens was tested for the presence of antibodies that bind to fentanyl or its analogs. Analysis was performed by either competitive binding ELISA or BLI to determine either IC₅₀ or K_{d} for fentanyl or its analogs. Results are shown in Table 11.

Vaccine Performance. A qualitative assessment of vaccine performance was conducted using a simple 0-3 scoring system for various parameters such as efficacy in reducing drug distribution to the brain, antinociception, respiratory depression, and bradycardia. Results are shown in Table 12. Because only F₁-CRM₁ was tested in the fentanyl self-administration (FSA) test, this parameter was not included in this overall evaluation of vaccine candidates. Based on these scores and the additional proof of efficacy for the F₆ conjugate in the 1 mg/kg fentanyl challenge (FIG. 12), the F₁-CRM₁, F₁-CRM₂, F₂-sKLH and F₆-CRM₂ adsorbed on alum adjuvant were identified as lead candidates in terms of in vivo efficacy against fentanyl and sufentanil.

TABLE 11
Off-target opioids

| Vaccine | | Buprenorphine | Naloxone | Naltrexone | Methadone |
|---|---|---|---|---|---|
| Formulation | Coating | IC₅₀ (mM)* | IC₅₀ (mM) | IC₅₀ (mM) | IC₅₀ (mM) |
| F₁-sKLH | F₁-BSA | >0.3 | >10 | >10 | >10 |
| F₂-sKLH | F₁-BSA | >0.3 | 0.1, 1 | 1, >10 | >10, 1.1 |
| F₃-sKLH | F₁-BSA | >0.3 | 9.2, >10 | >10 | >10 |
| F₄-sKLH | F₁-BSA | >0.3 | >10 | >10 | >10 |
| F₅-sKLH | F₁-BSA | >0.3 | >10 | >10 | >16 |
| F₆-CRM₂ | F₁-BSA | >0.3 | >10 | 1.9, 2 | >10 |

*The maximal concentration of buprenorphine tested was 0.3 mM because of the commercially available drug stock concentration. The maximal concentrations of naloxone, naltrexone, and methadone were 10 mM.

TABLE 12
Qualitative characterization of in vivo efficacy of conjugates containing the F₁₋₆ hapten series.

| | | | | | Heart | | Heart | | | |
|---|---|---|---|---|---|---|---|---|---|---|
| | Drug | Drug | | SaO₂% | Rate | SaO₂% | Rate | | | |
| | Increase | Decrease | Fentanyl | (0.075 | (0.075 | (0.1 | (0.1 | Sufentanil | Total | |
| Vaccine | [Serum] | [Brain] | MPE % | mg/kg) | mg/kg) | mg/kg) | mg/kg) | MPE % | Score | FSA |
| F₁-sKLH | 2 | 2 | 1 | 3 | 1 | 1 | 1 | 0 | 11 | — |
| F₁-CRM₁ | — | 3 | 3 | 3 | 3 | 3 | 3 | 1 | 19 | 2 |
| F₁-CRM₂ | — | 3 | 3 | 3 | 3 | 3 | 3 | 3 | 21 | — |
| F₂-sKLH | — | 3 | 3 | 3 | 3 | 3 | 3 | 3 | 21 | — |
| F₃-sKLH | — | 3 | 3 | 3 | 3 | 3 | 3 | 1 | 19 | — |
| F₃-CRM₂* | — | 3 | 3 | — | — | — | — | — | 6 | — |
| F₄-sKLH | 0 | 1 | 3 | — | — | 3 | 3 | 2 | 12 | — |
| F₅-sKLH | 2 | 2 | 3 | — | — | 3 | 3 | 2 | 15 | — |
| F₆-CRM₂** | 3 | 3 | 3 | — | — | 3 | 3 | 3 | 18 | — |

Scoring: — = Not tested; 0 = no effect; 1 = low effect; 2 = medium effect; 3 = high effect.
F₁₋₆ haptens were conjugated to subunit KLH (sKLH), and either CRM₁ (E. coli expressed CRM from Fina Biosolutions) or CRM₂ (PFEnex) depending on product availability. Scoring excludes FSA, as only one conjugate was used for those experiments. Symbol:
*Indicates conjugates tested only in mice.
Note:
sufentanil oximetry scores for SaO₂% and heart rate were 0 and therefore not included in this table.
**effective against fentanyl-induced breath rate reduction after 1 mg/kg fentanyl challenge (FIG. 12)

TABLE 5A
% MPE

| | | | | | |
|---|---|---|---|---|---|
| C | F₁ | F₈ | F₉ₐ | F_{9b} | F₁₀ |

Fentanyl (15 min)

| | | | | | | |
|---|---|---|---|---|---|---|
| C | | **** | ns | **** | *** | ** |
| F₁ | *** | | *** | ns | ns | ns |
| F₈ | ns | ** | | *** | ** | ns |
| F₉ₐ | *** | ns | ** | | ns | ns |
| F_{9b} | *** | ns | ** | ns | | ns |
| F₁₀ | *** | ns | ** | ns | ns | |

Fentanyl (30 min)
Fentanyl (45 min)

| | | | | | | |
|---|---|---|---|---|---|---|
| C | | *** | ns | *** | *** | ** |
| F₁ | * | | ns | ns | ns | ns |
| F₈ | ns | ** | | ns | ns | ns |
| F₉ₐ | * | ns | ** | | ns | ns |
| F_{9b} | * | ns | ** | ns | | ns |
| F₁₀ | ns | ns | * | ns | ns | |

Fentanyl (60 min)

TABLE 5B
Oxygen saturation

| | | | | | |
|---|---|---|---|---|---|
| C | F₁ | F₈ | F₉ₐ | F_{9b} | F₁₀ |

Fentanyl (15 min)

| | | | | | | |
|---|---|---|---|---|---|---|
| C | | **** | ** | **** | *** | *** |
| F₁ | **** | | ns | ns | ns | ns |
| F₈ | *** | ns | | ns | ns | ns |
| F₉ₐ | **** | ns | ns | | ns | ns |
| F_{9b} | **** | ns | ns | ns | | ns |
| F₁₀ | **** | ns | ns | ns | ns | |

Fentanyl (30 min)
Fentanyl (45 min)

| | | | | | | |
|---|---|---|---|---|---|---|
| C | **** | | ns | ns | ns | ns |
| F₁ | *** | ns | | ns | ns | ns |
| F₈ | **** | ns | ns | | ns | ns |
| F₉ₐ | **** | ns | ns | ns | | ns |
| F_{9b} | **** | ns | ns | ns | ns | |
| F₁₀ | ns | ns | ns | ns | ns | |

Fentanyl (60 min)

TABLE 5C
Heart rate

| | | | | | |
|---|---|---|---|---|---|
| C | F₁ | F₈ | F₉ₐ | F_{9b} | F₁₀ |

Fentanyl (15 min)

| | | | | | | |
|---|---|---|---|---|---|---|
| C | | * | ns | ** | * | ns |
| F₁ | *** | | ** | ns | ns | ns |
| F₈ | * | ns | | ** | * | ns |
| F₉ₐ | **** | ns | ns | | ns | ns |
| F_{9b} | *** | ns | ns | ns | | ns |
| F₁₀ | ** | ns | ns | ns | ns | |

Fentanyl (30 min)
Fentanyl (45 min)

| | | | | | | |
|---|---|---|---|---|---|---|
| C | | ns | ns | ** | ** | ** |
| F₁ | ns | | ns | ns | ns | ns |
| F₈ | ns | ns | | ns | ns | ns |
| F₉ₐ | ns | ns | ns | | ns | ns |
| F_{9b} | ns | ns | ns | ns | | ns |
| F₁₀ | ns | ns | ns | ns | ns | |

Fentanyl (60 min)

TABLE 5D
% MPE

| | | | | | |
|---|---|---|---|---|---|
| C | F₁ | F₈ | F₉ₐ | F_{9b} | F₁₀ |

Sufentanil (15 min)

| | | | | | | |
|---|---|---|---|---|---|---|
| C | | | ns | ns | ns | ns |
| F₁ | ns | | ns | ns | ns | ns |
| F₈ | ns | ns | | ns | ns | ns |
| F₉ₐ | ns | ns | ns | | ns | ns |
| F_{9b} | ns | ns | ns | ns | | ns |
| F₁₀ | ns | ns | ns | ns | ns | |

Sufentanil (30 min)
Sufentanil (45 min)

| | | | | | | |
|---|---|---|---|---|---|---|
| C | | ns | ns | ns | ns | ns |
| F₁ | ns | | ns | ns | ns | ns |
| F₈ | ns | ns | | ns | ns | ns |
| F₉ₐ | ns | ns | ns | | ns | ns |
| F_{9b} | ns | ns | ns | ns | | ns |
| F₁₀ | ns | ns | ns | ns | ns | |

Sufentanil (60 min)

TABLE 5E
Oxygen saturation

| | | | | | |
|---|---|---|---|---|---|
| C | F₁ | F₈ | F₉ₐ | F_{9b} | F₁₀ |

Sufentanil (15 min)

| | | | | | | |
|---|---|---|---|---|---|---|
| C | | ns | ns | ns | ns | ns |
| F₁ | ns | | ns | ns | ns | ns |
| F₈ | ns | ns | | ns | ns | ns |
| F₉ₐ | ns | ns | ns | | ns | ns |
| F_{9b} | ns | ns | ns | ns | | ns |
| F₁₀ | ns | ns | ns | ns | ns | |

Sufentanil (30 min)
Sufentanil (45 min)

| | | | | | | |
|---|---|---|---|---|---|---|
| C | | ** | * | ** | ns | ns |
| F₁ | ns | | ns | ns | ns | ns |
| F₈ | ns | ns | | ns | ns | ns |
| F₉ₐ | ns | ns | ns | | ns | ns |
| F_{9b} | ns | ns | ns | ns | | ns |
| F₁₀ | ns | ns | ns | ns | ns | |

Sufentanil (60 min)

TABLE 5F
Heart rate

| | | | | | |
|---|---|---|---|---|---|
| C | F₁ | F₈ | F₉ₐ | F_{9b} | F₁₀ |

Sufentanil (15 min)

| | | | | | | |
|---|---|---|---|---|---|---|
| C | | ns | ns | ns | ns | ns |
| F₁ | ns | | ns | ns | ns | ns |
| F₈ | ns | ns | | ns | ns | ns |
| F₉ₐ | * | ns | ns | | ns | ns |
| F_{9b} | * | ns | ns | ns | | ns |
| F₁₀ | * | ns | ns | ns | ns | |

Sufentanil (30 min)
Sufentanil (45 min)

| | | | | | | |
|---|---|---|---|---|---|---|
| C | | ns | ns | ** | ** | ** |
| F₁ | ns | | ns | ns | ns | ns |
| F₈ | ns | ns | | ns | ns | ns |
| F₉ₐ | ns | ns | ns | | ns | ns |
| F_{9b} | ns | ns | ns | ns | | ns |
| F₁₀ | ns | ns | ns | ns | ns | |

Sufentanil (60 min)

TABLE 6A
% MPE

| | | | | | |
|---|---|---|---|---|---|
| C | F₁ | F₈ | F₉ₐ | F_{9b} | F₁₀ |

Alfentanil (15 min)

| | | | | | | |
|---|---|---|---|---|---|---|
| C | | ns | ns | ns | ns | ns |
| F₁ | ns | | ns | ns | ns | ns |
| F₈ | ns | ns | | ns | ns | ns |
| F₉ₐ | ns | ns | ns | | ns | ns |
| F_{9b} | ns | ns | ns | ns | | ns |
| F₁₀ | ns | ns | ns | ns | ns | |

Alfentanil (30 min)
Alfentanil (45 min)

| | | | | | | |
|---|---|---|---|---|---|---|
| C | | ns | ns | ns | ns | ns |
| F₁ | ns | | ns | ns | ns | ns |
| F₈ | ns | ns | | ns | ns | ns |
| F₉ₐ | ns | ns | ns | | ns | ns |
| F_{9b} | ns | ns | ns | ns | | ns |
| F₁₀ | ns | ns | ns | ns | ns | |

Alfentanil (60 min)

TABLE 6B
Oxygen saturation

| | | | | | |
|---|---|---|---|---|---|
| C | F₁ | F₈ | F₉ₐ | F_{9b} | F₁₀ |

Alfentanil (15 min)

| | | | | | | |
|---|---|---|---|---|---|---|
| C | | ns | ns | ns | ns | ns |
| F₁ | ns | | ns | ns | ns | ns |
| F₈ | ns | ns | | ns | ns | ns |
| F₉ₐ | ns | ns | ns | | ns | ns |
| F_{9b} | ns | ns | ns | ns | | ns |
| F₁₀ | ns | ns | ns | ns | ns | |

Alfentanil (30 min)
Alfentanil (45 min)

| | | | | | | |
|---|---|---|---|---|---|---|
| C | | ns | ns | ns | ns | ns |
| F₁ | ns | | ns | ns | ns | ns |
| F₈ | ns | ns | | ns | ns | ns |
| F₉ₐ | ns | ns | ns | | ns | ns |
| F_{9b} | ns | ns | ns | ns | | ns |
| F₁₀ | ns | ns | ns | ns | ns | |

Alfentanil (60 min)

TABLE 6C
Heart rate

| | | | | | |
|---|---|---|---|---|---|
| C | F₁ | F₈ | F₉ₐ | F_{9b} | F₁₀ |

Alfentanil (15 min)

| | | | | | | |
|---|---|---|---|---|---|---|
| C | | ns | ns | ns | ns | ns |
| F₁ | ns | | ns | ns | ns | ns |
| F₈ | ns | ns | | ns | ns | ns |
| F₉ₐ | ns | ns | ns | | ns | ns |
| F_{9b} | ns | ns | ns | ns | | ns |
| F₁₀ | ns | ns | ns | ns | ns | |

Alfentanil (30 min)
Alfentanil (45 min)

| | | | | | | |
|---|---|---|---|---|---|---|
| C | | ns | ns | ns | ns | ns |
| F₁ | ns | | ns | ns | ns | ns |
| F₈ | ns | ns | | ns | ns | ns |
| F₉ₐ | ns | ns | ns | | ns | ns |
| F_{9b} | ns | ns | ns | ns | | ns |
| F₁₀ | * | * | ns | ns | ns | |

Alfentanil (60 min)

TABLE 7A
% MPE

| | | | | | |
|---|---|---|---|---|---|
| C | F₁ | F₈ | F₉ₐ | F_{9b} | F₁₀ |

Acetylfentanyl (15 min)

| | | | | | | |
|---|---|---|---|---|---|---|
| C | | ns | ns | ns | ns | ns |
| F₁ | ns | | ns | ns | ns | ns |
| F₈ | ns | ns | | ns | ns | ns |
| F₉ₐ | ns | ns | ns | | ns | ns |
| F_{9b} | ns | ns | ns | ns | | ns |
| F₁₀ | ns | ns | ns | ns | ns | |

Acetylfentanyl (30 min)
Acetylfentanyl (45 min)

| | | | | | | |
|---|---|---|---|---|---|---|
| C | | ns | ns | ns | ns | ns |
| F₁ | ns | | ns | ns | ns | ns |
| F₈ | ns | ns | | ns | ns | ns |
| F₉ₐ | ns | ns | ns | | ns | ns |
| F_{9b} | ns | ns | ns | ns | | ns |
| F₁₀ | ns | ns | ns | ns | ns | |

Acetylfentanyl (60 min)

TABLE 7B
Oxygen saturation

| | | | | | |
|---|---|---|---|---|---|
| C | F₁ | F₈ | F₉ₐ | F_{9b} | F₁₀ |

Acetylfentanyl (15 min)

| | | | | | | |
|---|---|---|---|---|---|---|
| C | | ns | ns | ns | ns | ns |
| F₁ | ns | | ns | ns | ns | ns |
| F₈ | ns | ns | | ns | ns | ns |
| F₉ₐ | ns | ns | ns | | ns | ns |
| F_{9b} | ns | ns | ns | ns | | ns |
| F₁₀ | ns | ns | ns | ns | ns | |

Acetylfentanyl (30 min)
Acetylfentanyl (45 min)

| | | | | | | |
|---|---|---|---|---|---|---|
| C | | ns | ns | ns | ns | ns |
| F₁ | ns | | ns | ns | ns | ns |
| F₈ | ns | ns | | ns | ns | ns |
| F₉ₐ | ns | ns | ns | | ns | ns |
| F_{9b} | ns | ns | ns | ns | | ns |
| F₁₀ | ns | ns | ns | ns | ns | |

Acetylfentanyl (60 min)

TABLE 7C
Heart rate

| | | | | | |
|---|---|---|---|---|---|
| C | F₁ | F₈ | F₉ₐ | F_{9b} | F₁₀ |

Acetylfentanyl (15 min)

| | | | | | | |
|---|---|---|---|---|---|---|
| C | | ns | ns | ns | ns | ** |
| F₁ | ns | | ns | ns | ns | ns |
| F₈ | ns | ns | | * | * | *** |
| F₉ₐ | ns | ns | ns | | ns | ns |
| F_{9b} | ns | ns | * | ns | | ns |
| F₁₀ | * | ns | * | ns | ns | |

Acetylfentanyl (30 min)
Acetylfentanyl (45 min)

| | | | | | | |
|---|---|---|---|---|---|---|
| C | | ns | ns | ns | ns | ns |
| F₁ | ns | | ns | ns | ns | ns |
| F₈ | ns | ns | | ns | ns | ns |
| F₉ₐ | ns | ns | ns | | ns | ns |
| F_{9b} | ns | ns | ns | ns | | ns |
| F₁₀ | ns | ns | * | ns | ns | |

Acetylfentanyl (60 min)

TABLE 7D
% MPE

| | | |
|---|---|---|
| C | F₁ | F₁₀ |

Acetylfentanyl (15 min)

| | | | |
|---|---|---|---|
| C | | * | * |
| F₁ | ns | | ns |
| F₁₀ | * | ns | |

Acetylfentanyl (30 min)
Acetylfentanyl (45 min)

| | | | |
|---|---|---|---|
| C | | ns | ns |
| F₁ | ns | | ns |
| F₁₀ | ns | ns | |

Acetylfentanyl (60 min)

TABLE 7E
Oxygen saturation

| | | |
|---|---|---|
| C | F₁ | F₁₀ |

Acetylfentanyl (15 min)

| | | | |
|---|---|---|---|
| C | | ns | * |
| F₁ | ns | | ns |
| F₁₀ | ns | ns | |

Acetylfentanyl (30 min)
Acetylfentanyl (45 min)

| | | | |
|---|---|---|---|
| C | | * | ** |
| F₁ | ns | | ns |
| F₁₀ | ns | ns | |

Acetylfentanyl (60 min)

TABLE 7F
Heart rate

| | | |
|---|---|---|
| C | F₁ | F₁₀ |

Acetylfentanyl (15 min)

| | | | |
|---|---|---|---|
| C | | *** | *** |
| F₁ | ** | | ns |
| F₁₀ | ** | ns | |

Acetylfentanyl (30 min)
Acetylfentanyl (45 min)

| | | | |
|---|---|---|---|
| C | | ns | * |
| F₁ | ns | | ns |
| F₁₀ | ns | ns | |

Acetylfentanyl (60 min)

TABLE 8A
% MPE

| | | | |
|---|---|---|---|
| C | F₁ | F₁₁ | F₁₃ |

Carfentanil (15 min)

| | | | | |
|---|---|---|---|---|
| C | | ns | ns | ns |
| F₁ | ns | | ns | ns |
| F₁₁ | ns | ns | | ns |
| F₁₃ | ns | ns | ns | |

Carfentanil (30 min)
Carfentanil (45 min)

| | | | | |
|---|---|---|---|---|
| C | | ns | ns | ns |
| F₁ | ns | | ns | ns |
| F₁₁ | ns | ns | | ns |
| F₁₃ | ns | ns | ns | |

Carfentanil (60 min)

TABLE 8B
Oxygen saturation

| | | | |
|---|---|---|---|
| C | F₁ | F₁₁ | F₁₃ |

Carfentanil (15 min)

| | | | | |
|---|---|---|---|---|
| C | | ns | ns | ** |
| F₁ | ns | | ns | ** |
| F₁₁ | ns | * | | ** |
| F₁₃ | ns | ns | ns | |

Carfentanil (30 min)
Carfentanil (45 min)

| | | | | |
|---|---|---|---|---|
| C | | ns | ns | ns |
| F₁ | ns | | ns | ns |
| F₁₁ | ns | ns | | ns |
| F₁₃ | ns | * | * | |

Carfentanil (60 min)

TABLE 8C
Heart rate

| | | | |
|---|---|---|---|
| C | F₁ | F₁₁ | F₁₃ |

Carfentanil (15 min)

| | | | | |
|---|---|---|---|---|
| C | | ns | ns | ns |
| F₁ | ns | | ns | ns |
| F₁₁ | ns | ns | | ns |
| F₁₃ | ns | ns | ns | |

Carfentanil (30 min)
Carfentanil (45 min)

| | | | | |
|---|---|---|---|---|
| C | | ns | ns | ns |
| F₁ | ns | | ns | ns |
| F₁₁ | ns | ns | | ns |
| F₁₃ | ns | ns | ns | |

Carfentanil (60 min)

TABLE 9A
% MPE

| | | | |
|---|---|---|---|
| C | F₁ | F₁₁ | F₁₃ |

Fentanyl (15 min)

| | | | | |
|---|---|---|---|---|
| C | | **** | ns | ns |
| F₁ | **** | | **** | **** |
| F₁₁ | ns | **** | | ns |
| F₁₃ | ns | * | ns | |

Fentanyl (30 min)
Fentanyl (45 min)

| | | | | |
|---|---|---|---|---|
| C | | **** | ns | ns |
| F₁ | ** | | ** | ns |
| F₁₁ | * | ns | | ns |
| F₁₃ | * | ns | ns | |

Fentanyl (60 min)

TABLE 9B
Oxygen saturation

| | | | |
|---|---|---|---|
| C | F₁ | F₁₁ | F₁₃ |

Fentanyl (15 min)

| | | | | |
|---|---|---|---|---|
| C | | ** | ns | * |
| F₁ | ns | | * | ns |
| F₁₁ | ns | ns | | ns |
| F₁₃ | ns | ns | ns | |

Fentanyl (30 min)
Fentanyl (45 min)

| | | | | |
|---|---|---|---|---|
| C | | ns | ns | ns |
| F₁ | ns | | ns | ns |
| F₁₁ | ns | ns | | ns |
| F₁₃ | ns | ns | ns | |

Fentanyl (60 min)

TABLE 9C
Heart rate

| | | | |
|---|---|---|---|
| C | F₁ | F₁₁ | F₁₃ |

Fentanyl (15 min)

| | | | | |
|---|---|---|---|---|
| C | | ns | ns | ns |
| F₁ | ns | | ns | ns |
| F₁₁ | ns | ns | | ns |
| F₁₃ | ns | ns | ns | |

Fentanyl (30 min)
Fentanyl (45 min)

| | | | | |
|---|---|---|---|---|
| C | | **** | ns | ns |
| F₁ | **** | | **** | **** |
| F₁₁ | ns | **** | | ns |
| F₁₃ | ns | * | ns | |

Fentanyl (60 min)

TABLE 10A
% MPE

| | | | |
|---|---|---|---|
| C | F₁ | F₁₁ | F₁₃ |

Combo (15 min)

| | | | | |
|---|---|---|---|---|
| C | | ns | ns | ns |
| F₁ | ns | | ns | ns |
| F₁₁ | ns | ns | | ns |
| F₁₃ | ns | ns | ns | |

Combo (30 min)
Combo (45 min)

| | | | | |
|---|---|---|---|---|
| C | | ns | ns | ns |
| F₁ | ns | | ns | ns |
| F₁₁ | ns | ns | | ns |
| F₁₃ | ns | ns | ns | |

Combo (60 min)

TABLE 10B
Oxygen saturation

| | | | |
|---|---|---|---|
| C | F₁ | F₁₁ | F₁₃ |

Combo (15 min)

| | | | | |
|---|---|---|---|---|
| C | | ns | ns | ns |
| F₁ | ns | | ns | ns |
| F₁₁ | ns | ns | | ns |
| F₁₃ | ns | ns | ns | |

Combo (30 min)
Combo (45 min)

| | | | | |
|---|---|---|---|---|
| C | | ns | ns | ns |
| F₁ | ns | | ns | ns |
| F₁₁ | ns | ns | | ns |
| F₁₃ | ns | ns | ns | |

Combo (60 min)

TABLE 10C
Heart rate

| | | | |
|---|---|---|---|
| C | F₁ | F₁₁ | F₁₃ |

Combo (15 min)

| | | | | |
|---|---|---|---|---|
| C | | ns | ns | ns |
| F₁ | ns | | ns | ns |
| F₁₁ | ns | ns | | ns |
| F₁₃ | ns | ns | ns | |

Combo (30 min)
Combo (45 min)

| | | | | |
|---|---|---|---|---|
| F₁ | | ns | ns | ns |
| F₁₁ | ns | | ns | ns |
| F₁₃ | ns | ns | | ns |
| C | ns | ns | ns | |

Combo (60 min)

TABLE 11A
% MPE

| | | | |
|---|---|---|---|
| C | F₁ | F₁₁ | F₁₃ |

Carfentanil (5 ug/kg)

| | | | | |
|---|---|---|---|---|
| C | | ns | **** | **** |
| F₁ | ns | | * | * |
| F₁₁ | ns | ns | | ns |
| F₁₃ | *** | *** | ns | |

Carfentanil (10 ug/kg)
Carfentanil (15 ug/kg)

| | | | | |
|---|---|---|---|---|
| C | C | F1 | F11 | F13 |
| F₁ | | ns | ns | *** |
| F₁₁ | ns | | ns | *** |
| F₁₃ | ns | ns | | ns |
| C | ns | ns | ns | |

Carfentanil (20 ug/kg)

TABLE 11A
Oxygen saturation

| | | | |
|---|---|---|---|
| C | F₁ | F₁₁ | F₁₃ |

Carfentanil (5 ug/kg)

| | | | | |
|---|---|---|---|---|
| C | | ns | ns | ns |
| F₁ | ns | | * | ns |
| F₁₁ | ns | ns | | ns |
| F₁₃ | ns | ns | ns | |

Carfentanil (10 ug/kg)
Carfentanil (15 ug/kg)

| | | | | |
|---|---|---|---|---|
| C | | ns | * | * |
| C | ns | | ns | ** |
| F₁ | ns | ns | | ns |
| F₁₁ | ns | ** | ns | |

Carfentanil (20 ug/kg)

TABLE 11C
Heart rate

| | | | |
|---|---|---|---|
| C | F₁ | F₁₁ | F₁₃ |

Carfentanil (5 ug/kg)

| | | | | |
|---|---|---|---|---|
| C | | ns | ns | ns |
| F₁ | ns | | ns | ns |
| F₁₁ | ns | ns | | ns |
| F₁₃ | ns | ns | ns | |

Carfentanil (10 ug/kg)
Carfentanil (15 ug/kg)

| | | | | |
|---|---|---|---|---|
| C | | ns | ns | ns |
| F₁ | ns | | ns | ns |
| F₁₁ | ns | ns | | ns |
| F₁₃ | ns | ns | ns | |

Carfentanil (20 ug/kg)

The complete disclosure of all patents, patent applications, and publications, and electronically available material cited herein are incorporated by reference. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

1. A fentanyl hapten comprising

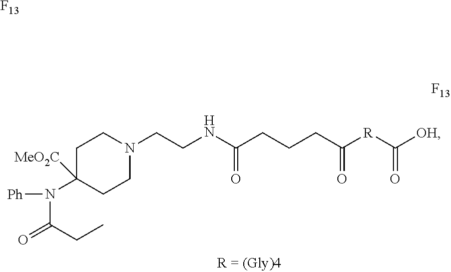

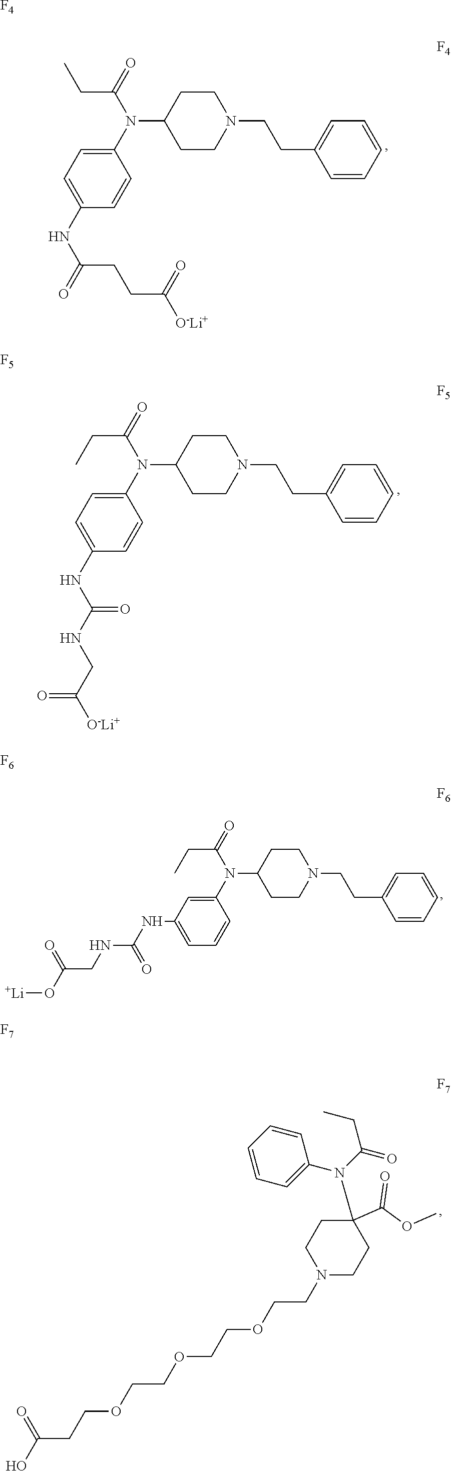

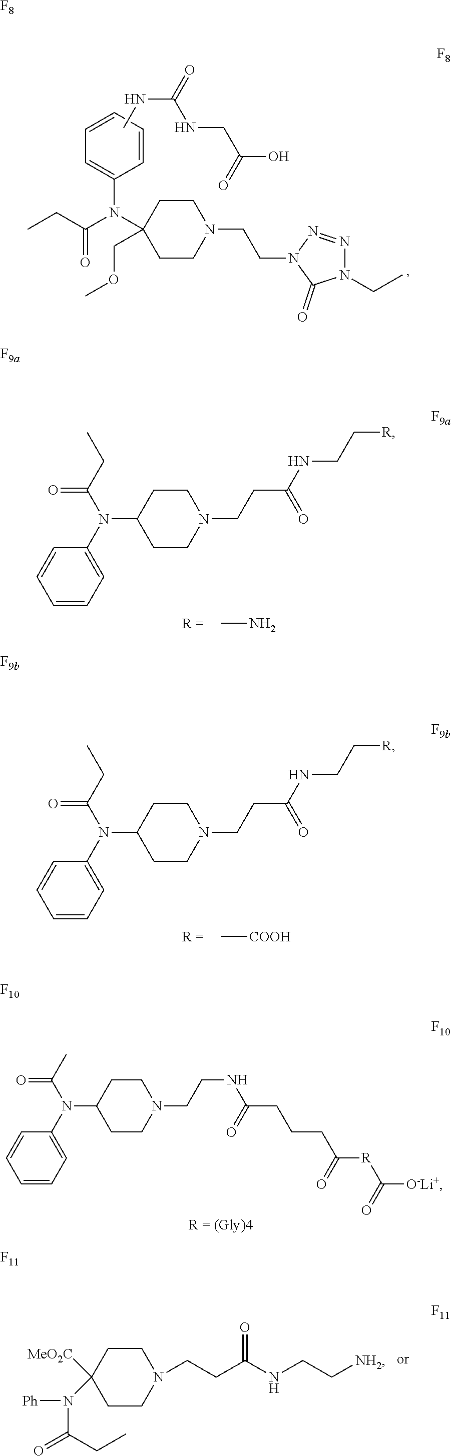

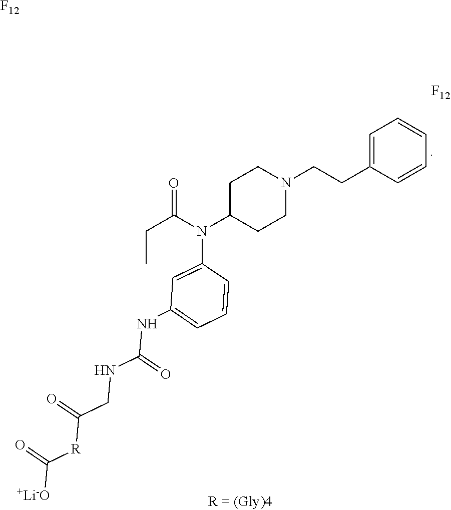

2. A fentanyl hapten-carrier conjugate comprising
a fentanyl hapten comprising 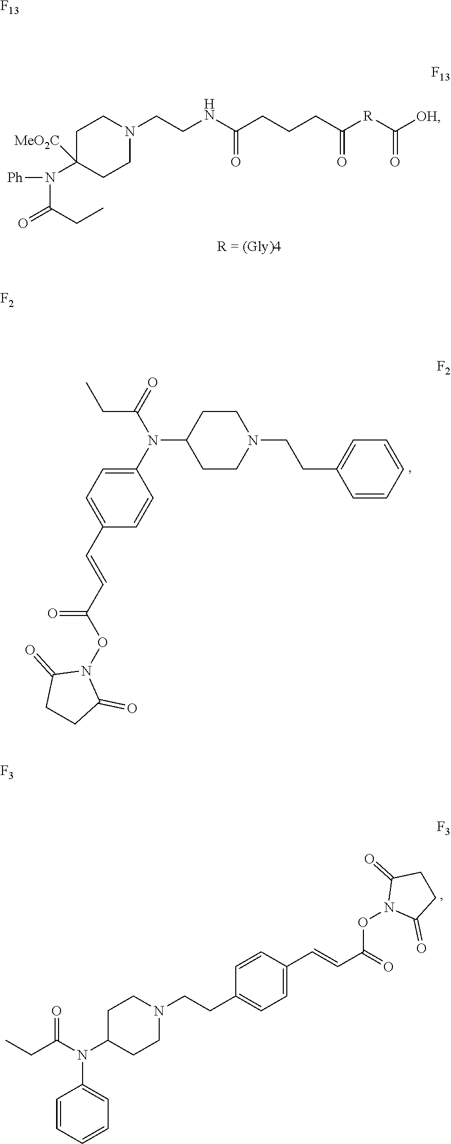 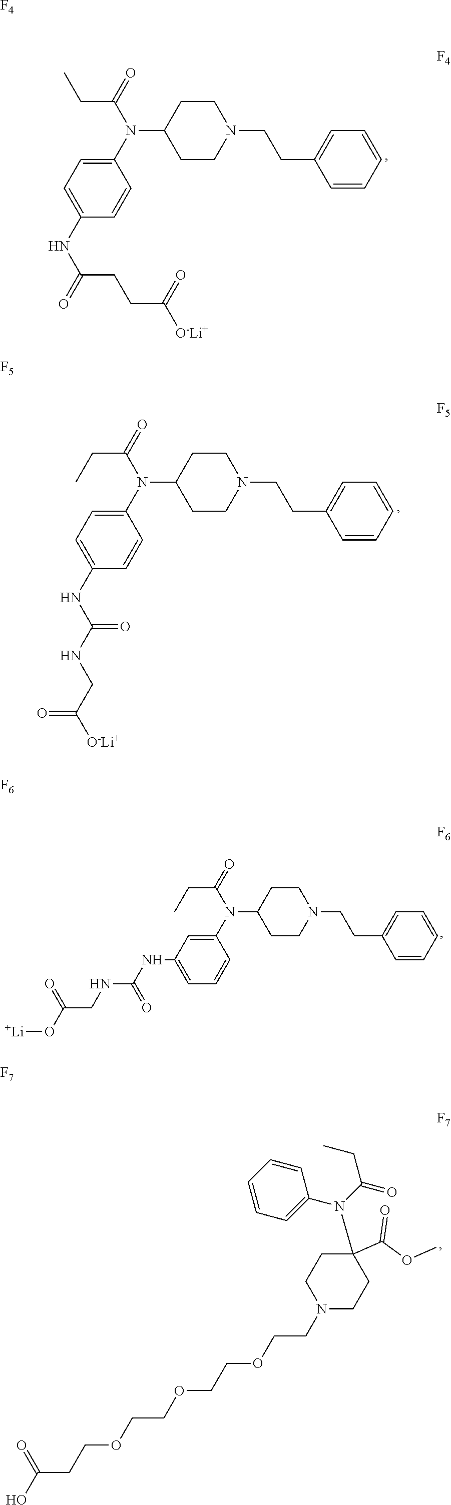 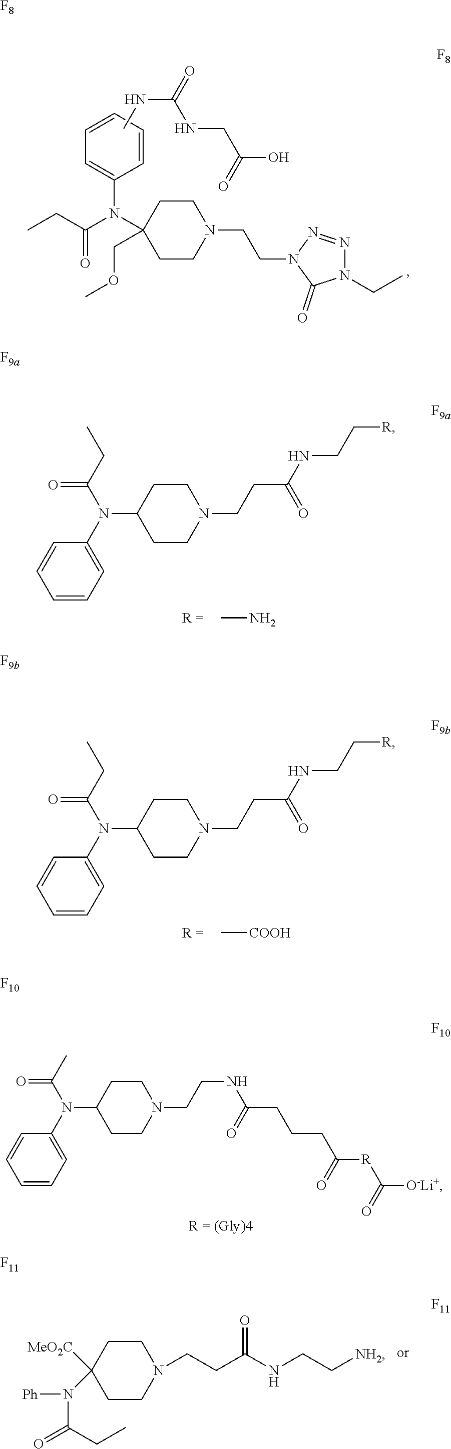 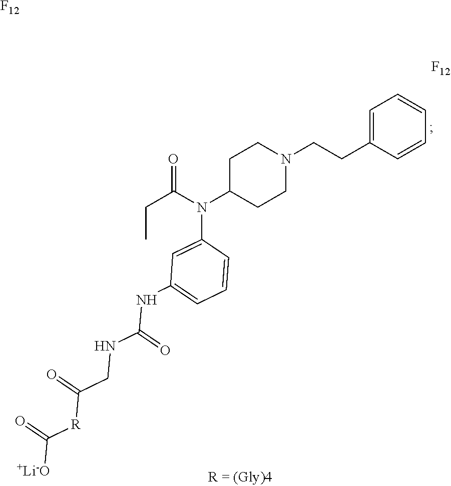
an immunogenic carrier, wherein the fentanyl hapten is conjugated to the immunogenic carrier.

3. The fentanyl hapten-carrier conjugate of claim 2, wherein the immunogenic carrier comprises CRM.

4. The fentanyl hapten-carrier conjugate of claim 2, wherein the number of fentanyl hapten molecules per immunogenic carrier molecule is at least 1, at least 5, at least 10, at least 15, at least 20, at least 30, at least 40, or at least 50.

5. A composition comprising the fentanyl hapten of claim 1.

6. A composition comprising the fentanyl hapten-carrier conjugate of claim 2.

7. A method of making the fentanyl hapten of claim 1.

8. A method of making the fentanyl hapten-carrier conjugate of claim 2.

9. A method of administering the fentanyl hapten of claim 1 to a subject.

10. A method of administering the fentanyl hapten-carrier conjugate of claim 2 to a subject.

11. A method of administering the composition of claim 5 to a subject.

12. A method of administering the composition of claim 6 to a subject.

13. The method of claim 10, wherein the subject is an individual at risk of exposure to fentanyl, a fentanyl derivative, or a fentanyl analog.

14. The method of claim 10, wherein the subject is an individual who has been diagnosed with an opioid use disorder.

15. The method of claim 10, wherein the subject is an individual who has been diagnosed with a substance use disorder.
